(19) United States
(12) Patent Application Publication
(10) Pub. No.: US 2024/0345106 A1
(43) Pub. Date: Oct. 17, 2024

(54) Chronic Disease Management Test to Inform Clinical Care of Patients with Chronic Conditions

(71) Applicant: Aegis Sciences Corporation, Nashville, TN (US)

(72) Inventors: Joshua SCHRECKER, Franklin, TN (US); Rebecca HELTSLEY, Hendersonville, TN (US); David SCHWOPE, Hendersonville, TN (US); Christopher WESTERFIELD, Brentwood, TN (US)

(73) Assignee: Aegis Sciences Corporation, Nashville, TN (US)

(21) Appl. No.: 18/634,737

(22) Filed: Apr. 12, 2024

Related U.S. Application Data
(60) Provisional application No. 63/496,153, filed on Apr. 14, 2023.
(60) Provisional application No. 63/573,157, filed on Apr. 2, 2024.

(51) Int. Cl. G01N 33/94; A61B 10/00

(52) U.S. Cl. CPC G01N 33/94; A61B 10/0051; G01N 2800/52

(57) ABSTRACT

The present disclosure provides methods for using a chronic disease management (CDM) test to evaluate, diagnose, manage, facilitate, and inform clinical care of patients with chronic conditions, by providing a reliable and convenient diagnostic test for detecting medication nonadherence (NA) and drug-drug interactions (DDIs).

### CROSS-REFERENCE TO RELATED APPLICATION

This application claims the benefit of priority of U.S. Provisional Application Nos. 63/496,153 filed Apr. 14, 2023, and 63/573,157 filed Apr. 2, 2024, each of which is herein incorporated by reference in its entirety.

### FIELD OF THE INVENTION

The present disclosure provides methods for using a chronic disease management (CDM) test to evaluate, diagnose, manage, facilitate, and inform clinical care of patients by detecting medication nonadherence (NA) and drug-drug interactions (DDIs).

### DESCRIPTION OF THE RELATED ART

Treating chronic conditions such as cardiometabolic diseases relies on sustained efforts ranging from lifestyle modification, public health measures, and procedural interventions to achieve better health outcomes. The mainstay of chronic condition treatment and secondary prevention, however, is poly-pharmacologic therapy. Clinical management of patients with chronic disease, such as cardiometabolic disease, is complicated by polypharmacy, defined as the concomitant administration of five or more medications (Delara et al., “Prevalence and factors associated with polypharmacy: a systematic review and meta-analysis,” BMC Geriatr 22, 601 (2022); Davies et al., “Adverse outcomes of polypharmacy in older people: systematic review of reviews,” J Am Med Dir Assoc. 21(2):181-187 (2020)). The prevalence of polypharmacy among adults in the United States (U.S.) is highest among those with heart disease and has risen dramatically from 40.6% (95% confidence interval [C], 34.5-46.7) between 1999-2000 to 61.7% (95% CI, 55.2-68.2) between 2017-2018 (Wang et al., “Prevalence and trends of polypharmacy in U.S. adults, 1999-2018,” Glob Health Res Policy July 12; 8(1):25 (2023)). Consequently, when clinicians are confronted with diagnosing the etiology of a patient's clinical deterioration, they are challenged to distinguish polypharmacy-related factors, such as NA and DDI, from the natural progression of chronic disease.

The physician, already challenged to prescribe the appropriate medication at the correct dose, must also hope that patients take their prescribed medication. Numerous studies indicate >40% of patients with chronic conditions do not correctly follow their prescribed drug regimens, which is due to many factors including the sheer volume of medications they are prescribed (Kleinsinger et al., “The Unmet Challenge of Medication Nonadherence,” The Permanente Journal 22:18-033 (2018); Xu et al., “Tailored Interventions to Improve Medication Adherence for Cardiovascular Diseases,” Frontiers in Pharmacology 11 (2020); Marcum et al., “Medication Nonadherence: A Diagnosable and Treatable Medical Condition,” JAMA: the journal of the American Medical Association 309(20):2105 (2013): Vyas et al., “Chronic Condition Clusters and Polypharmacy among Adults,” International Journal of Family Medicine 2012:1-8 (2012)). With this level of polypharmacy, patients are at increased risk of NA and for DDIs. Researchers found the majority of the drugs implicated in DDIs are prescribed for chronic conditions, and specifically for cardiometabolic diseases.

Medication nonadherence rates in a recent meta-analysis ranged from 7.0%-83.5%, with the highest rates among adults taking medications for cardiovascular disease and diabetes (Foley et al., “Prevalence and predictors of medication non-adherence among people living with multimorbidity: a systematic review and meta-analysis,” BMJ Open September 2: 11(9):e044987 (2021): Wong et al, “The association between multimorbidity and poor adherence with cardiovascular medications,” Int J Cardiol. December 15; 177(2):477-82 (2014); Vatcharavongvan et al., “Polypharmacy, medication adherence and medication management at home in elderly patients with multiple non-communicable diseases in Thai primary care,” Fam Med Prin Care Rev 19:412-6. 10.5114/fmpcr.2017.70818 (2017)). This systematic review of 178 studies revealed that medication nonadherence was detected better, albeit incompletely, by self-report (76.5%) than by reliance on pharmacy data (69.4%) or electronic monitoring (44.1%), further evidence that the detection of medication nonadherence remains a diagnostic challenge. For example, measurements of drugs or drug metabolites in body fluid (most commonly blood or urine) and the direct observation of drug ingestion accurately detect systemic drug presence, however, these methods may be invasive or impractical for patients with multiple comorbidities and/or polypharmacy (Lam et al., “Medication Adherence Measures: An Overview,” Biomed Res Int. 2015:217047 (2015)). Additionally, indirect detection methods, such as monitoring indicators in pharmacy records and other electronic systems, make it more difficult for patients to falsify adherence; however, patients may fill the prescriptions or engage the devices and still not ingest the medications. Consequently, enhanced efforts to obtain accurate data are needed because medication nonadherence has been shown to not only impact quality and length of life, but also account for approximately 50% of clinical treatment failures, 125,000 deaths, and as many as 25% of annual U.S. hospitalizations (Kim et al., “Medication Adherence: The Elephant in the Room,” MEDICATiON MANAG EMENT US Pharm. 43(1)30-34 (2018)).

The risk of DDIs also increases with polypharmacy. Adverse drug reactions (ADRs) have been estimated to cost the U.S. healthcare system $30.1 billion annually, with recent reports estimating that approximately 18% of ADRs are due to DDIs (Sultana et al., “Clinical and economic burden of adverse drug reactions,” J Pharmacol Pharmacother. December; 4(Suppl 1):S73-7 (2013); Jiang et al., “Adverse drug reactions and correlations with drug-drug interactions: A retrospective study of reports from 2011 to 2020,” Front Pharmacol. August 22; 13:923939 (2022); Magro et al., “Identifying and Characterizing Serious Adverse Drug Reactions Associated With Drug-Drug Interactions in a Spontaneous Reporting Database,” Front Pharmacol. January 18; 11:622862 (2021)). Current approaches to detecting DDIs include electronic databases and expert consensus evidence-based decision support (Roblek et al., “Drug-drug interaction software in clinical practice: a systematic review,” Eur J Cin Pharmacol. February; 71(2):131-42 (2015); Scheife et al., “Consensus recommendations for systematic evaluation of drug-drug interaction evidence for clinical decision support,” Drug Saf. February; 38(2):197-206 (2015)). Although certain software programs can provide accurate evaluation of potentially harmful DDIs, their utility has been limited in clinical practice for various reasons, including an inability to determine the clinical appropriateness of the potentially harmful drug combinations (Kheshti et al., “A comparison of five common drug-drug interaction software programs regarding accuracy and comprehensiveness,” Journal of research in pharmacy practice 5(4), 257 (2016); Sheikh-Taha et al, “Polypharmacy and severe potential drug-drug interactions among older adults with cardiovascular disease in the United States,” BMC Geriatr 21, 233 (2021)). To this end, an expert consensus panel developed a transparent and systematic evidence-based process for evaluating DDIs to support clinical decision-making (Scheife et al., “Consensus recommendations for systematic evaluation of drug-drug interaction evidence for clinical decision support,” Drug Saf. February; 38(2):197-206 (2015)). Despite being successful in several aspects, they determined that more work is needed to identify clinical relevance of the DDI, such as severity grading; factors that predict patient harm; and a quality of evidence rating system.

Physicians and other providers must not only disentangle NA from DDIs but also separate these two issues from disease progression. Moreover, the busy physician must sort through these three possibilities in a dynamic, time-constrained clinical setting. Unsurprisingly, NA and DDIs are overlooked at best and, at worse, attributed to disease progression.

An important reason physicians have difficulty in distinguishing NA from DDIs and disease progression is lack of an effective and standardized method to test for these conditions. While point-of-care drug alerts have the potential to prevent DDIs, they have had limited success, with as many as 98% of these alerts overridden (McEvoy et al., “Variation in high-priority drug-drug interaction alerts across institutions and electronic health records,” Journal of the American Medical Informatics Association 24(2):331-8 (2017)). There remains a need for an effective and standardized system to address these issues in polypharmacy patients and a provider- and patient-friendly approach to accurately distinguish NA, DDIs, and disease progression.

### SUMMARY OF THE INVENTION

In an aspect, the present disclosure provides for, and includes, methods for using a CDM test to evaluate, diagnose, manage, facilitate, and inform clinical care of patients with chronic conditions.

In an aspect, the present disclosure provides, and includes, a method for treating a chronic disease in a patient in need thereof, comprising: obtaining an oral fluid sample from the patient; analyzing the oral fluid for analytes of one or more drug classes selected from the group consisting of antiarrhythmics; antidepressants and antipsychotics; antiemetics and gastric reflux therapies; antiepileptics; antimicrobials and antivirals; cardiovascular; chemotherapeutic agents; cognitive enhancement agents; diabetes mellitus (DM); diabetes mellitus type 2 (DM2); nonsteroidal anti-inflammatory drugs (NSAIDs); skeletal muscle relaxant; foods and supplements; and miscellaneous drugs; identifying a list of drugs recently ingested by the patient based on the analyzing step; determining whether any drug-drug interaction (DDI) is present between the drugs in the list; and if DDI is present, presenting a first treatment plan to the patient; or if DDI is not present, presenting a second treatment plan to the patient.

In an aspect, the present disclosure provides, and includes, a method for improving a symptom of a chronic disease in a patient in need thereof, comprising: obtaining an oral fluid sample from the patient; analyzing the oral fluid for analytes of one or more drug classes selected from the group consisting of antiarrhythmics; antidepressants and antipsychotics; antiemetics and gastric reflux therapies; antiepileptics; antimicrobials and antivirals; cardiovascular; chemotherapeutic agents; cognitive enhancement agents; diabetes mellitus (DM); diabetes mellitus type 2 (DM2); nonsteroidal anti-inflammatory drugs (NSAIDs); skeletal muscle relaxant; foods and supplements; and miscellaneous drugs; identifying a list of drugs recently ingested by the patient based on the analyzing step; determining that a drug-drug interaction (DDI) is present between the drugs in the list; and determining an intervention, wherein the intervention comprises decreasing or increasing the dosage or discontinuing administration of one or more drugs determined as interacting with another drug in the list, and continuing administration of the remaining drugs in the list to the patient; wherein the symptom is improved after the intervention in comparison to prior to the intervention.

In an aspect, the present disclosure provides, and includes, a method for treating a chronic disease in a patient in need thereof, comprising: obtaining an oral fluid sample from the patient; analyzing the oral fluid for analytes of one or more drug classes selected from the group consisting of asthma/chronic obstructive pulmonary disease (COPD); cardiovascular; diabetes mellitus (DM); diabetes mellitus type 2 (DM2); and chronic kidney disease (CKD); identifying a list of drugs recently ingested by the patient based on the analyzing step; determining if there is medication nonadherence (NA) in the list of drugs by generating a list of drugs with NA and a list of drugs without NA; and if NA is present, presenting a first treatment plan to the patient; or if NA is not present, presenting a second treatment plan to the patient.

In an aspect, the present disclosure provides, and includes, a method of improving a symptom of a chronic disease in a patient in need thereof, comprising: obtaining an oral fluid sample from the patient; analyzing the oral fluid for analytes of one or more drug classes selected from the group consisting of asthma/chronic obstructive pulmonary disease (COPD); cardiovascular; diabetes mellitus (DM); diabetes mellitus type 2 (DM2); and chronic kidney disease (CKD); identifying a list of drugs recently ingested by the patient based on the analyzing step; determining that there is medication nonadherence (NA) in the list of drugs by generating a list of drugs with NA and a list of drugs without NA; and perform the step of: decreasing or increasing the dosage or discontinuing administration of one or more drugs with NA, continuing administration of the list of drugs without NA to the patient; wherein the symptom is improved after performing the step in comparison to prior to performing the step.

In an aspect, the present disclosure provides, and includes, a method for improving management of a chronic disease in a patient in need thereof, comprising the steps of: i. obtaining an oral fluid sample from the patient; ii. analyzing the oral fluid for analytes of one or more drug classes; iii. determining whether a drug-drug interaction (DDI) is present based on step ii; iv. determining an intervention based on the results from steps ii-iii, wherein if DDI is present, the intervention comprises: a. counseling the patient on DDI; b. documenting DDI in the patient's chart; c. documenting a newly identified adverse drug effect and/or therapeutic non-response in the patient's chart; d. adjusting or discontinuing a medication that is causing DDI; e. utilizing a new or maximizing an existing non-pharmacologic treatment option; and f. using a monitoring plan for potential increased symptoms of DDI; wherein if DDI is not present, the intervention comprises: g. counseling the patient on disease progression or other underlying issues related to adverse drug reaction (ADR); h. maintaining or adjusting the patient's current medication regimen; and i. using a monitoring plan for potential increased symptoms attributed to disease progression or other underlying issues related to ADR; and v. presenting the intervention to the patient; wherein the management of the chronic disease is improved after the intervention in comparison to prior to the intervention.

In an aspect, the present disclosure provides, and includes, a method for improving management of a chronic disease in a patient in need thereof, comprising the steps of: i. obtaining an oral fluid sample from the patient; ii. analyzing the oral fluid for analytes of one or more drug classes; iii. determining whether one or more medication nonadherence (NA) is present based on step ii, wherein each of the one or more NA corresponds to an analyte abnormality; iv. determining an intervention based on the results from steps ii-iii, wherein if NA is present, the intervention comprises: a. counseling the patient on NA; b. documenting NA on the patient's chart; c. continuing, adjusting, or discontinuing a medication that is causing NA; d. utilizing a tool to mitigate NA; and e. treating a secondary condition that may be the potential cause of NA; wherein if NA is not present, the intervention comprises: f. counseling the patient on disease progression or other underlying issues related to adverse drug reaction (ADR); g. maintaining or adjusting the patient's current medication regimen; h. using a monitoring plan for potential increased symptoms attributed to disease progression or other underlying issues related to ADR; and v. presenting the intervention to the patient; wherein the management of the chronic disease is improved after the intervention in comparison to prior to the intervention.

### BRIEF DESCRIPTION OF THE DRAWINGS

Some aspects of the disclosure are herein described, by way of example only, with reference to the accompanying drawings. With specific reference now to the drawings in detail, it is stressed that the particulars shown are by way of example and are for purposes of illustrative discussion of aspects of the disclosure. In this regard, the description and the drawings, considered alone and together, make apparent to those skilled in the art how aspects of the disclosure may be practiced.

FIG. 1 discloses a sample collection and CDM testing scheme according to the present disclosure.

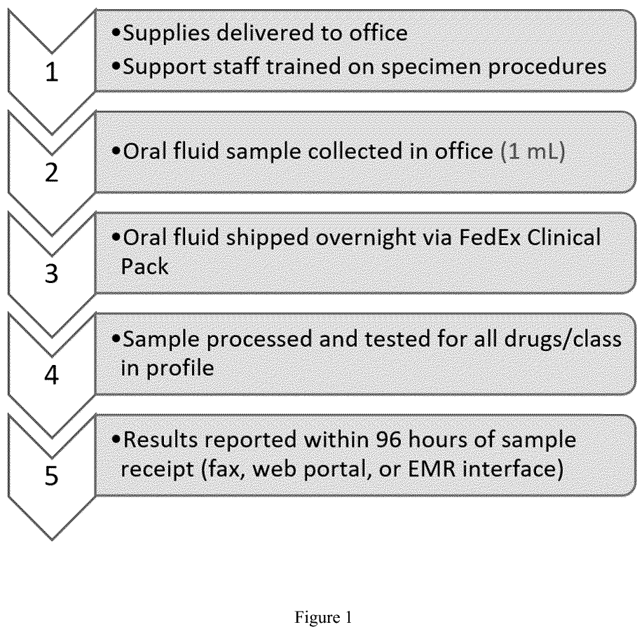

FIG. 2 depicts a diagram illustrating physician enrollment procedures.

FIG. 3 depicts a diagram illustrating intervention patient data procedures.

FIG. 4 depicts a diagram illustrating control patient data procedures.

FIG. 5 depicts the percent of participants, by study arm, that correctly identify medication nonadherence (MNA), DDI, and disease progression.

### DETAILED DESCRIPTION

The present disclosure describes methods for using a CDM test to evaluate, diagnose, manage, facilitate, and inform clinical care of patients with chronic conditions.

This description is not intended to be a detailed catalog of all the different ways in which the disclosure may be implemented, or all the features that may be added to the instant disclosure. For example, features illustrated with respect to one embodiment may be incorporated into other embodiments, and features illustrated with respect to a particular embodiment may be deleted from that embodiment. Thus, the disclosure contemplates that in some embodiments of the disclosure, any feature or combination of features set forth herein can be excluded or omitted. In addition, numerous variations and additions to the various embodiments suggested herein will be apparent to those skilled in the art in light of the instant disclosure, which do not depart from the instant disclosure. In other instances, well-known structures, interfaces and processes have not been shown in detail in order not to unnecessarily obscure the invention. It is intended that no part of this specification be construed to effect a disavowal of any part of the full scope of the invention. Hence, the following descriptions are intended to illustrate some particular embodiments of the disclosure, and not to exhaustively specify all permutations, combinations, and variations thereof.

Unless otherwise defined, all technical and scientific term used herein have the same meaning as commonly understood by one of ordinary skill in the art to which this disclosure belongs. The terminology used in the description of the disclosure herein is for the purpose of describing particular aspects or embodiments only and is not intended to be limiting of the disclosure.

All publications, patent applications, patents and other references cited herein are incorporated by reference in their entireties for the teachings relevant to the sentence and/or paragraph in which the reference is presented. References to techniques employed herein are intended to refer to the techniques as commonly understood in the art, including variations on those techniques or substitutions of equivalent techniques that would be apparent to one of skill in the art.

Unless the context indicates otherwise, it is specifically intended that the various features of the disclosure described herein can be used in any combination. Moreover, the present disclosure also contemplates that in some embodiments of the disclosure, any feature or combination of features set forth herein can be excluded or omitted.

The methods disclosed herein include and comprise one or more steps or actions for achieving the described method. The method steps and/or actions may be interchanged with one another without departing from the scope of the present invention. In other words, unless a specific order of steps or actions is required for proper operation of the embodiment, the order and/or use of specific steps and/or actions may be modified without departing from the scope of the present invention.

As used in the description of the disclosure and the appended claims, the singular forms “a,” “an” and “the” are intended to include the plural forms as well, unless the context clearly indicates otherwise.

As used herein, “and/or” refers to and encompasses any and all possible combinations of one or more of the associated listed items, as well as the lack of combinations when interpreted in the alternative (“or”).

The terms “about” and “approximately” as used herein when referring to a measurable value such as a length, a frequency, or a SEM value and the like, is meant to encompass variations of ±20%, ±10%, ±5%, ±1%, ±0.5%, or even ±0.1% of the specified amount.

As used herein, phrases such as “between X and Y” and “between about X and Y” should be interpreted to include X and Y. As used herein, phrases such as “between about X and Y” mean “between about X and about Y” and phrases such as “from about X to Y” mean “from about X to about Y.”

As used herein a “patient” may be a human or animal subject. The terms “patient” and “subject” are intended to be interchangeable. As used herein, a patient may be an animal (e.g., without being limiting, a mammal, reptile, bird, fish, amphibian) or other organism, such as, without being limiting, a plant or fungus. In an aspect, the patient can be a healthy individual, an individual that has or is suspected of having a disease or a predisposition to the disease, or an individual that is in need of therapy or suspected of needing therapy. In an aspect, the patient has or is suspected of having a chronic disease, disorder, or condition. In an aspect, the patient is a human adult. In an aspect, the patient is a geriatric individual.

As used herein, a “sample” may be obtained from the patient. In an aspect, a sample comprises a cell. In an aspect, a sample comprises a tissue. In an aspect, a sample comprises an organ. In an aspect, a sample comprises blood. In an aspect, a sample comprises plasma. In an aspect, a sample comprises urine. In an aspect, a sample comprises feces. In an aspect, a sample comprises a bodily fluid. In an aspect, a sample comprises an oral fluid. In an aspect, a sample comprises an oral fluid which is saliva. Additional non-limiting examples of samples include serum, sputum, semen, vaginal fluid, synovial fluid, spinal fluid, sweat, tears, nasal fluid, and saliva.

In an aspect, a sample provided herein comprises about 1 mL of oral fluid. In an aspect, a sample provided herein comprises about 2 mL of oral fluid. In an aspect, a sample provided herein comprises about 3 mL of oral fluid. In an aspect, a sample provided herein comprises about 3 mL of oral fluid. In an aspect, a sample provided herein comprises about 4 mL of oral fluid. In an aspect, a sample provided herein comprises about 5 mL of oral fluid.

In an aspect, a sample is obtained in a doctor's office. In an aspect, a sample is obtained in a clinical care center. In an aspect, a sample is obtained in a long-term care facility. In an aspect, a sample is obtained at a laboratory or testing facility. In an aspect, a sample is obtained at a patient's home. In an aspect, a sample is obtained in a nursing home. In an aspect, a sample is obtained in a hospital. In an aspect, a sample is obtained in a pharmacy.

As used herein, “CDM” refers to chronic disease management.

As used herein, “NA” or “MNA” refers to medication nonadherence. NA occurs when a patient does not take their medication as recommended from a healthcare provider (e.g., doctor, nurse, caregiver). In an aspect, medication as recommended from a health care providers is communicated and provided to a patient in one or more prescriptions. In an aspect, NA comprises a patient not taking one or more prescribed medications. In an aspect, NA comprises a patient taking one or more medications not prescribed by a healthcare provider. In an aspect, NA comprises a patient taking one or more medications at the incorrect frequency, time, or dosage. In an aspect, an NA corresponds to an analyte abnormality. In an aspect, an analyte abnormality can refer to a finding that is not in alignment with the patient's treatment regimen. In an aspect, an analyte abnormality is classified into categories of (i) prescribed to the patient but not detected by CDM test, or (ii) detected in CDM test but not prescribed to the patient.

As used herein, “drug” and “medication” are used interchangeably.

As used herein, “DDI” refers to drug-drug interaction, and “DDIs” refers to drug-drug interactions. In an aspect, DDIs occur when two or more drugs interact with each other, leading to additive, synergistic, or antagonistic pharmacological effects. In an aspect, DDIs occur between a drug and a food or supplement. In an aspect, DDIs can influence drug efficacy, lead to adverse effects, and result in poor treatment outcome.

As used herein, “ADR” refers to adverse drug reaction, and “ADRs” refers to adverse drug reactions. In an aspect, ADR is an unintended reaction resulting from the use of one or more drugs for the prophylaxis, diagnosis, or treatment of a disease in a patient. In an aspect, an ADR is associated with a DDI. In an aspect, an ADR is associated with NA. In an aspect, an ADR is associated with an underlying issue that is unrelated to DDIs. In an aspect, an ADR is associated with an underlying issue that is unrelated to NA.

As used herein, “AND” refers to adherent, no drug-drug interaction.

As used herein, “HF” refers to heart failure, and “CHF” refers to chronic heart failure.

As used herein, “D” and “DM2” refer to diabetes mellitus and diabetes mellitus type 2, respectively.

As used herein, “HTN” refers to hypertension.

As used herein, “Afib” refers to atrial fibrillation.

As used herein, “NSAIDs” refers to nonsteroidal anti-inflammatory drugs.

As used herein, “CKD” refers to chronic kidney disease.

As used herein, “CHD” refers to coronary heart disease or chronic heart disease.

As used herein, “CVD” refers to cardiovascular disease.

As used herein, “CAD” refers to coronary artery disease.

As used herein, “COPD” refers to chronic obstructive pulmonary disease.

As used herein, “MI” refers to myocardial infarction.

As used herein, “EKG” refers to electrocardiograms.

As used herein, “CPV” refers to Clinical Performance and Value. In an aspect, CPV vignettes are simulations of patient cases and scenarios used to evaluate and compare clinical practices of healthcare providers in a comprehensive range of clinical conditions.

As used herein, “DxTx” refers to diagnosis and treatment. In an aspect, a DxTx score measures how well a healthcare providers performed in making the correct diagnosis and providing the correct treatment.

As used herein, “O.R.” refers to odds ratio. In an aspect, an odds ratio is a statistic that quantifies the strength of the association between two events, A and B.

As used herein, “PCP” refers to primary care physician.

As used herein, “APP” refers to advanced practice providers.

As used herein, “RCT” refers to randomized controlled trial.

As used herein, “LC-MS/MS” refers to liquid chromatography-tandem mass spectrometry.

As used herein, a “chronic disease” refers to any long-term condition that requires ongoing medical treatment to manage. In an aspect, a chronic disease is selected from the group consisting of asthma/COPD, cardiovascular diseases (e.g., CHD, CAD, HF, CHF, deep vein thrombosis and pulmonary embolism), DM, DM2, and CKD. In an aspect, a patient has 1, 2, 3, 4, or 5 or more chronic diseases concurrently.

As used herein, the term “treat” or “treating” refers to completely or partially improving a disease, disorder, or condition, completely or partially reducing the severity of the one or more symptoms of a disease, disorder, or condition, or completely or partially preventing the occurrence or recurrence of a disease, disorder, or condition in a patient who may be predisposed to the disease, disorder, or condition, but has not yet been diagnosed with said disease, disorder, or condition. In an aspect, treatment refers to managing or improving the management of a disease, disorder, or condition.

In an aspect, treatment refers to therapeutic, prophylactic, or preventative measures. The terms “treatment” and “intervention” are intended to be interchangeable.

In an aspect, a treatment comprises increasing the dosage of one or more drugs. In an aspect, a treatment comprises decreasing the dosage of one or more drugs. In an aspect, a treatment comprises discontinuing administration of one or more drugs. In an aspect, a treatment comprises discontinuing administration of one or more drugs with NA or DDI. In an aspect, a treatment comprises titrating the dosage of one or more drugs. In an aspect, a treatment comprises substituting one or more existing drugs with NA or DDI with one or more new drugs. In an aspect, a treatment comprises administering one or more new drugs to the patient. In an aspect, a treatment comprises advising the patient about the importance of adherence. In an aspect, a treatment comprises providing aids and tools to increase adherence of drugs with NA. In an aspect, a treatment comprises updating an existing treatment plan or implementing a new treatment plan to allow for monitoring of the patient's symptom onset or resolution. In an aspect, a treatment plan comprises providing a patient with one or more follow-up monitoring tests. In an aspect, a treatment plan comprises providing a patient with one or more follow-up cardiac monitoring tests. In an aspect, a treatment plan comprises providing a patient with one or more follow-up electrocardiograms (EKGs). In an aspect, a treatment plan comprises one or more further assessments of the presenting signs and symptoms experienced by the patient.

As used herein, a “list of drugs” refers to drugs recently ingested by a patient and detected by the CDM test. In an aspect, a list of drugs comprises prescription drugs. In an aspect, a list of drugs comprises prescription drugs, non-prescription drugs, and other miscellaneous substances. In an aspect, a patient is administered 1, 2, 3, 4, 5, 6, 7, 8, 9, or 10 or more drugs concurrently. In an aspect, a patient recently ingested 1, 2, 3, 4, 5, 6, 7, 8, 9, or 10 or more drugs concurrently prior to the CDM test. In an aspect, a patient is administered 1, 2, 3, 4, 5, 6, 7, 8, 9, or 10 or more drugs concurrently after implementation of an intervention as determined based on results from the CDM test. In an aspect, the CDM test detects drugs ingested by a patient in the past 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 32, 34, 36, 38, 40, 42, 44, 46, 48, 50, 60, 70, 80, 90, or 100 hours. In an aspect, the CDM test detects drugs ingested by a patient in the past 48 hours.

As used herein, “ingested” by a patient refers to drugs taken-up into the body of the patient. In an aspect, drugs are ingested orally via inhaled medications. In an aspect, drugs are ingested via orally administered solid, liquid, or aerosolized medications. In an aspect, drugs are ingested by a patient via non-oral administration routes, for example, intravenous, intramuscular, subcutaneous, rectal, vaginal, transdermal, or topical.

As used herein, the term “analyte” refers to the drug parent compound or metabolites of the parent compound detected by the CDM test. In an aspect, the CDM test detects between 10 and 50 analytes, between 50 and 100 analytes, between 100 and 150 analytes, between 150 and 200 analytes, between 200 and 250 analytes, or between 250 and 300 analytes. In an aspect, the CDM test detects analytes from between 1 and 5 drug classes, between 5 and 10 drug classes, between 10 and 15 drug classes, or between 15 and 20 drug classes.

In an aspect, the present disclosure provides for, and includes, a CDM test that detects analytes of one or more drug classes selected from the group consisting of, antiarrhythmics, antidepressants and antipsychotics, antiemetics and gastric reflux therapies, antiepileptics; antihypertensives, antimicrobials and antivirals, antithrombotics, asthma/COPD, cardiovascular, chemotherapeutic agents, cognitive enhancement agents, DM, DM2, inhaled corticosteroids and beta agonists, NSAIDs, skeletal muscle relaxant, steroids, CKD, foods and supplements, and miscellaneous drugs.

In an aspect, the present disclosure provides for, and includes, a CDM test that further detects analytes of one or more drug classes selected from the group consisting of amphetamines, anti-Parkinson agents, benzodiazepines, opiates, opioids, and mixed agonists-antagonists.

In an aspect, the present disclosure provides for, and includes, a kit comprising reagents for detecting analytes from one or more drug classes selected from the group consisting of antiarrhythmics, antidepressants and antipsychotics, antiemetics and gastric reflux therapies, antiepileptics; antihypertensives, antimicrobials and antivirals, antithrombotics, asthma/COPD, cardiovascular, chemotherapeutic agents, cognitive enhancement agents, DM, DM2, inhaled corticosteroids and beta agonists, NSAIDs, skeletal muscle relaxant, steroids, CKD, foods and supplements, and miscellaneous drugs.

In an aspect, the present disclosure provides for, and includes, a kit comprising reagents for further detecting analytes from one or more drug classes selected from the group consisting of amphetamines, anti-Parkinson agents, benzodiazepines, opiates, opioids, and mixed agonists-antagonists.

In an aspect, a kit comprises reagents for detecting between 10 and 50 analytes, between 50 and 100 analytes, between 100 and 150 analytes, between 150 and 200 analytes, between 200 and 250 analytes, or between 250 and 300 analytes. In an aspect, a kit comprises reagents for detecting analytes from between 1 and 5 drug classes, between 5 and 10 drug classes, between 10 and 15 drug classes, or between 15 and 20 drug classes.

In an aspect, for detecting DDIs, analytes of the antiarrhythmics drug class are selected from the group consisting of amiodarone, desethylamiodarone, quinidine, ranolazine, and a combination thereof.

In an aspect, for detecting DDIs, analytes of the antidepressants and antipsychotic drug class are selected from the group consisting of citalopram, N-desmethylcitalopram, duloxetine, fluvoxamine, fluoxetine, norfluoxetine, iloperidone, lurasidone, nefazodone, olanzapine, paroxetine, 7-hydroxy quetiapine, norquetiapine, quetiapine, 9-hydroxyrisperidone, norsertraline, sertraline, trazodone, O-desmethylvenlafaxine, venlafaxine, vortioxetine, and a combination thereof.

In an aspect, for detecting DDIs, analytes of the antiemetic and gastric reflux drug class are selected from the group consisting of aprepitant, cimetidine, fosamprenavir, 4-hydroxy omeprazole sulfide, omeprazole sulfone, and a combination thereof.

In an aspect, for detecting DDIs, analytes of antiepileptics drug class are selected from the group consisting of butalbital, carbamazepine, carbamazepine epoxide, phenobarbital, phenytoin, primidone, and a combination thereof.

In an aspect, for detecting DDIs, analytes of the antimicrobials and antivirals drug class are selected from the group consisting of atazanavir, azithromycin, N-desmethyl azithromycin, ciprofloxacin, clarithromycin, cobicistat, darunavir, delavirdine, 8-hydroxyefavirenz, efavirenz, erythromycin, etravirine, fluconazole, amprenavir, indanavir, itraconazole, 2-hydroxyitraconazole, ketoconazole, levofloxacin, ofloxacin, metronidazole, 2-hydroxynelfinavir, nelfinavir, posaconazole, quinine, rifabutin, rifampin, rifapentine, hydroxy ritonavir, ritonavir, saquinavir, sulfamethoxazole, tipranavir, trimethoprim, voriconazole, voriconazole N-oxide, and a combination thereof.

In an aspect, for detecting DDIs, analytes of the cardiovascular drug class are selected from the group consisting of amlodipine, amlodipine metabolite, apixaban, salicylic acid, chlorothiazide, clonidine, clopidogrel, clopidogrel COOH, digoxin, deacetyl diltiazem-N-oxide, diltiazem, fenofibrate, fenofibrate metabolite, gemfibrozil, hydrochlorothiazide, lovastatin, lovastatin acid, rivaroxaban, sacubitril, simvastatin, simvastatin acid, ticagrelor, triamterene, norverapamil, verapamil, 7-hydroxywarfarin, warfarin, and a combination thereof.

In an aspect, for detecting DDIs, analytes of the chemotherapeutic agents drug class are selected from the group consisting of enzalutamide, nilotinib, pazopanib, and a combination thereof.

In an aspect, for detecting DDIs, analytes of the cognitive enhancement drug class are selected from the group consisting of 3-hydroxyguanfacine, guanfacine, and a combination thereof.

In an aspect, for detecting DDIs, analytes of the DM/DM2 drug class are selected from the group consisting of hydroxypioglitazone, pioglitazone, and a combination thereof.

In an aspect, for detecting DDIs, analytes of the NSAIDs drug class are selected from the group consisting of celecoxib, diclofenac, ibuprofen, indomethacin, ketorolac, meloxicam, naproxen, and a combination thereof.

In an aspect, for detecting DDIs, analytes of the skeletal muscle relaxant drug class are selected from the group consisting of cyclobenzaprine, norcyclobenzaprine, dehydrotizanidine, tizanidine, and combination thereof.

In an aspect, for detecting DDIs, analytes of the foods and supplements drug class are selected from the group consisting of bergaptol, dihydroxybergamottin, dihydrokavain, hyperforin, and a combination thereof.

In an aspect, for detecting DDIs, analytes of the miscellaneous drug class are selected from the group consisting of avanafil, colchicine, cyclosporine, 7-hydroxymethotrexate, methotrexate, tacrolimus, and a combination thereof.

In an aspect, for detecting NA, analytes of the asthma/chronic obstructive pulmonary disease (COPD) drug class are selected from the group consisting of Aclidinium, Albuterol/Levalbuterol, Arformoterol/Formoterol, Indacaterol, Olodaterol, Salmeterol, Vilanterol, Beclomethasone Monopropionate, Beclomethasone, Budesonide, Fluticasone Furoate, Fluticasone Propionate, Methylprednisolone, Roflumilast Metabolite, Roflumilast, Ipratropium, Tiotropium, Umeclidinium, and a combination thereof.

In an aspect, for detecting NA, analytes of the cardiovascular drug class are selected from the group consisting of Hydralazine Metabolite, Acebutolol, Amlodipine Metabolite, Amlodipine, Apixaban, Salicylic Acid, Atenolol, Atorvastatin Lactone, Atorvastatin, Azilsartan, Benazepril, Benazeprilat, Bendroflumethiazide, Betaxolol, Bisoprolol, Bumetanide, Candesartan, Captopril, Carvedilol, Chlorothiazide, Chlorthalidone, Clopidogrel COOH, Clopidogrel, Dabigatran, Deacetyl Diltiazem-N-Oxide, Diltiazem, Dipyridamole, Enalapril, Enalaprilat, Eplerenone, Eprosartan, Dehydro Felodipine, Felodipine, Fluvastatin, Fosinopril, Fosinoprilat, Furosemide, Hydrochlorothiazide, Indapamide, Irbesartan, Isradipine, Labetalol, Lisinopril, Losartan Metabolite, Losartan, Lovastatin acid, Lovastatin, Methyclothiazide, Metolazone, alpha-Hydroxymetoprolol, Metoprolol, Moexipril, Moexiprilat, Nadolol, Nebivolol, Nicardipine, Nifedipine Carboxylate, Nifedipine, Nisoldipine, Olmesartan, Perindopril, Pindolol, Pitavastatin, Pravastatin, Propranolol, Quinapril, Quinaprilat, Ramipril Metabolite, Ramipril, Rivaroxaban, Rosuvastatin, Sacubitril Metabolite, Sacubitril, Simvastatin, Simvastatin Acid, Canrenoic Acid, Canrenone, Spironolactone, Telmisartan, Ticagrelor, Timolol, Torsemide, Trandolapril, Trandolaprilat, Valsartan, Norverapamil, Verapamil, 7-Hydroxywarfarin, Warfarin, and a combination thereof.

In an aspect, for detecting NA, analytes of the DM/DM2 drug class are selected from the group consisting of Metformin (Glucophage), Linagliptin, Sitagliptin, Canagliflozin, Dapagliflozin, Empagliflozin, Glimepiride, Glipizide, Glyburide Metabolite, Glyburide, Hydroxypioglitazone, Pioglitazone, and a combination thereof.

In an aspect, analytes of the ACE inhibitor substance class are selected from the group consisting of Benazepril, Benazeprilat, Captopril, Enalapril, Enalaprilat, Fosinopril, Fosinoprilat, Lisinopril, Moexipril, Moexiprilat, Perindopril, Quinapril, Quinaprilat, Ramipril Metabolite, Ramipril, Trandolapril, Trandolaprilat, and a combination thereof.

In an aspect, analytes of the aldosterone antagonist substance class are selected from the group consisting of Eplerenone, Canrenoic Acid, Canrenone, Spironolactone, and a combination thereof.

In an aspect, analytes of the angiotensin receptor blocker substance class are selected from the group consisting of Azilsartan, Candesartan, Eprosartan, Irbesartan, Losartan Metabolite, Losartan, Olmesartan, Telmisartan, Valsartan, and a combination thereof.

In an aspect, analytes of the anticholinergic substance class are selected from the group consisting of Aclidinium, Ipratropium, Tiotropium, Umeclidinium, and a combination thereof.

In an aspect, analytes of the anticoagulants substance class are selected from the group consisting of Apixaban, Dabigatran, Rivaroxaban, 7-Hydroxywarfarin, Warfarin, and a combination thereof.

In an aspect, analytes of the antiplatelet substance class are selected from the group consisting of Salicylic Acid, Clopidogrel COOH, Clopidogrel, Dipyridamole, Ticagrelor, and a combination thereof.

In an aspect, analytes of the beta agonist substance class are selected from the group consisting of Albuterol/Levalbuterol, Arformoterol/Formoterol, Indacaterol, Olodaterol, Salmeterol, Vilanterol, and a combination thereof.

In an aspect, analytes of the beta blocker substance class are selected from the group consisting of Acebutolol, Atenolol, Betaxolol, Bisoprolol, Carvedilol, Labetalol, alpha-Hydroxymetoprolol, Metoprolol, Nadolol, Nebivolol, Pindolol, Propranolol, Timolol, and a combination thereof.

In an aspect, an analyte of the biguanide substance class is Metformin (Glucophage).

In an aspect, analytes of the calcium channel blocker substance class are selected from the group consisting of Amlodipine Metabolite, Amlodipine, Deacetyl Diltiazem-N-Oxide, Diltiazem, Dehydro Felodipine, Felodipine, Isradipine, Nicardipine, Nifedipine Carboxylate, Nifedipine, Nisoldipine, Norverapamil, Verapamil, and a combination thereof.

In an aspect, analytes of the corticosteroid substance class are selected from the group consisting of Beclomethasone Monopropionate, Beclomethasone, Budesonide, Fluticasone Furoate, Fluticasone Propionate, Methylprednisolone, and a combination thereof.

In an aspect, analytes of the DPP-4 inhibitor substance class are selected from the group consisting of Linagliptin, Sitagliptin, and a combination thereof.

In an aspect, analytes of the loop diuretic substance class are selected from the group consisting of Furosemide, Torsemide, Bumetanide, and a combination thereof.

In an aspect, analytes of the neprilysin inhibitor substance class are selected from the group consisting of Sacubitril Metabolite, Sacubitril, and a combination thereof.

In an aspect, analytes of the phosphodiesterase inhibitor substance class are selected from the group consisting of Roflumilast Metabolite, Roflumilast, and a combination thereof.

In an aspect, analytes of the sodium-glucose co-transporter 2 (SGLT2) inhibitor substance class are selected from the group consisting of Canagliflozin, Dapagliflozin, Empagliflozin, and a combination thereof.

In an aspect, analytes of the statin substance class are selected from the group consisting of Atorvastatin Lactone, Atorvastatin, Fluvastatin, Lovastatin acid, Lovastatin, Pitavastatin, Pravastatin, Rosuvastatin, Simvastatin, Simvastatin Acid, and a combination thereof.

In an aspect, analytes of the sulfonylureas substance class are selected from the group consisting of Glimepiride, Glipizide, Glyburide Metabolite, Glyburide, and a combination thereof.

In an aspect, analytes of the thiazide diuretics substance class are selected from the group consisting of Bendroflumethiazide, Chlorothiazide, Chlorthalidone, Hydrochlorothiazide, Indapamide, Methyclothiazide, Metolazone, and a combination thereof.

In an aspect, analytes of the thiazolidinedione substance class are selected from the group consisting of Hydroxypioglitazone, Pioglitazone, and a combination thereof.

In an aspect, an analyte of the vasodilator substance class is Hydralazine Metabolite.

As used herein, an “analyzing step” refers to the step of analyzing the sample (e.g., oral fluid) for the analytes. In an aspect, an analyzing step is performed by mass spectrometry. In an aspect, analytes in the CDM test are qualitatively evaluated using high-performance liquid chromatography-tandem mass spectrometry (LC-MS/MS). In an aspect, oral fluid samples collected with a commercial collection device are processed via automated Solid Phase Extraction (SPE) and extracts are diluted and injected onto an LC-MS/MS system. In an aspect, Modified C18 columns are used to chromatograph compounds and detection is via scheduled multiple reaction monitoring using electrospray with polarity switching.

In an aspect, an analyzing step identifies a list of drugs recently ingested by a patient.

As used herein, a “list of drugs with NA” refers to drugs not taken in accordance with the recommendation from a healthcare provider (e.g. physician). In an aspect, a list of drugs with NA comprises one or more drugs prescribed to a patient but not taken by the patient. In an aspect, a list of drugs with NA comprises one or more drugs prescribed to a patient but taken by the patient at the incorrect frequency, time, or dosage.

As used herein, a “list of drugs without NA” refers to drugs taken in accordance with the recommendation from a healthcare provider (e.g. physician). In an aspect, a list of drugs without NA comprises one or more drugs prescribed to a patient and taken by the patient. In an aspect, a list of drugs without NA comprises one or more drugs prescribed to a patient and taken by the patient in any amount. In an aspect, a list of drugs without NA comprises one or more drugs prescribed to a patient and taken by the patient according to the prescription instructions.

As used herein, a “prescription drug list” is a list of one or more drugs prescribed to a patient by a healthcare provider. In an aspect, a prescription drug list can also include non-prescription drugs or miscellaneous substances recommended as part of the treatment by a patient's healthcare provider.

In an aspect, a prescription drug list is provided by a healthcare provider at the same time as obtaining the sample from a patient. In an aspect, a prescription drug list is provided by a healthcare provider at the same time the CDM test is ordered. In an aspect, a prescription drug list is provided by a healthcare provider through fax, a website portal, email, or an electronic health record (EMR) interface. In an aspect, a prescription drug list is obtained from a patient's existing records. In an aspect, a prescription drug list is obtained from a database. In an aspect, a prescription drug list is stored on a database.

As used herein, “generating compliance calls” refers to assigning a list of drugs to a list of drugs with NA or a list of drugs without NA based on comparing against a prescription drug list. In an aspect, the comparing comprises determining if each and every drug in the prescription drug list is present in the list of drugs. In an aspect, the comparing comprises determining if each and every drug in the prescription drug list is present in the expected amount in the list of drugs. In an aspect, the expected amount of each drug in the prescription drug list is predicted using information derived from the prescription, including but not limited to dosage information and prescription length.

In an aspect, the comparing comprises determining if each and every drug in the prescription drug list is present in the list of drugs, and reporting drugs present on both the list of drugs and the prescription drug list as adherent (or compliant). In an aspect, a list of drugs without NA is generated, where the list of drugs without NA comprises one or more drugs present on both the list of drugs and the prescription drug list. In an aspect, no drugs are present on both the list of drugs and the prescription drug list, a list of drugs without NA is generated and comprises no drugs.

In an aspect, the comparing comprises determining if each and every drug in the prescription drug list is present in the list of drugs, and reporting drugs only present on the prescription drug list and not present on the list of drugs as NA (or non-compliant). In an aspect, a list of drugs with NA is generated, where the list of drugs with NA comprises one or more drugs present on the prescription drug list and not present on the list of drugs. In an aspect, all drugs present on the list of drugs are present on the prescription drug list, a list of drugs with NA is generated and comprises no drugs.

In an aspect, one or more drugs or miscellaneous substances not prescribed to a patient but taken by the patient is detected by the CDM test and reported as present. In an aspect, a second list of drugs is generated, where the second list of drugs comprises one or more drugs present on the list of drugs but not present on the prescription drug list. In an aspect, all drugs present on the prescription drug list are present on the list of drugs, a second list of drugs is generated and comprises no drugs.

In an aspect, determining whether any DDI is present between the drugs in the list of drugs comprises the following steps: (a) analytes that are found above a reporting threshold are reported as present; (b) analytes that are associated with a drug parent compound are mapped to a drug information database (e.g., a database licensed through First Databank (FDB)); (c) combinations of all detected compounds are assessed for associated drug-drug interactions within the drug information database; (d) if a drug-drug interaction associated with concurrent ingestion of one or more pairs of compounds is identified in the list of drug detected by the CDM test, the interaction is reported, wherein the report comprises (i) one or more interacting compounds/analytes, (ii) severity of the interaction as assigned by the drug information database, (iii) clinical information regarding the interaction as assigned by the drug information database. In an aspect, a reporting threshold is a predetermined value. In an aspect, a reporting threshold is a predetermined value individual to each analyte. In an aspect, a reporting threshold is a normalized value. In an aspect, a reporting threshold is derived from a standard.

In an aspect, an intervention is counseling a patient on DDI. In an aspect, an intervention is documenting DDI in a patient's chart. In an aspect, an intervention is documenting a newly identified adverse drug effect and/or therapeutic non-response in a patient's chart. In an aspect, an intervention is adjusting or discontinuing a medication that is causing DDI. In an aspect, an intervention is utilizing a new or maximizing an existing non-pharmacologic treatment option. In an aspect, an intervention is using a monitoring plan for potential increased symptoms of DDI. In an aspect, an intervention is counseling a patient on disease progression or other underlying issues related to ADR that is not DDI.

In an aspect, an intervention is maintaining or adjusting the patient's current medication regimen. In an aspect, an intervention is using a monitoring plan for potential increased symptoms attributed to disease progression or other underlying issues related to ADR that is not DDI.

In an aspect, an intervention is counseling a patient on NA. In an aspect, an intervention is documenting NA on the patient's chart. In an aspect, an intervention is continuing, adjusting, or discontinuing a medication that is causing NA. In an aspect, an intervention is utilizing a tool to mitigate NA. In an aspect, a tool to mitigate NA is a smart reminder. In an aspect, a tool to mitigate NA is an alarm setting. In an aspect, a tool to mitigate NA is a digital pill. In an aspect, an intervention is treating a secondary condition that may be the potential cause of NA. In an aspect, a secondary condition is depression. In an aspect, an intervention is counseling a patient on disease progression or other underlying issues related to ADR that is not NA. In an aspect, an intervention is maintaining or adjusting the patient's current medication regimen. In an aspect, an intervention is using a monitoring plan for potential increased symptoms attributed to disease progression or other underlying issues related to ADR that is not NA.

In an aspect, DDIs are classified into categories based on the severity of the DDIs. In an aspect, a DDI is classified as a moderate DDI. In an aspect, a DDI is classified as a severe DDI.

As used herein, the term “improving management of a chronic disease” can refer to reducing the severity or reducing the number of incidences of DDIs or NA in a patient with a chronic disease who was previously experiencing DDIs or NA. In an aspect, the management of a chronic disease is improved when there is a change in the severity of a DDI detected in a patient. In an aspect, the management of a chronic disease is improved when a DDI which was previously classified as severe is reduced to moderate. In an aspect, the management of a chronic disease is improved when there is a decrease in the number of DDIs detected in a patient. In an aspect, the management of a chronic disease is improved when there is a decrease of 1, 2, 3, 4, 5, 6, 7, 8, 9, or 10 or more DDIs detected in a patient. In an aspect, the management of a chronic disease is improved when there is a decrease in the number of NAs detected in a patient. In an aspect, the management of a chronic disease is improved when there is a decrease of 1, 2, 3, 4, 5, 6, 7, 8, 9, or 10 or more NAs detected in a patient. In an aspect, the management of a chronic disease is improved when lack of DDIs or NAs in a patient as detected by CDM test directs a healthcare provider (e.g., doctor, nurse, or caregiver) to interventions suitable for disease progression.

In an aspect, the management of a chronic disease is improved as assessed based on results from a subsequent CDM test. In an aspect, a subsequent CDM test is performed at a later time point compared to a prior CDM test. In an aspect, the time period between a prior CDM test and a subsequent CDM test is two weeks, three weeks, 1 month, 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, or 24 months. In an aspect, results from at least 2, at least 3, at least 4, at least 5, at least 6, at least 7, at least 8, at least 9, or at least 10 CDM tests are used to assess improvement in the management of a chronic disease.

In an aspect, reporting of CDM test results occurs within 5, 10, 12, 20, 24, 30, 26, 40, 50, 60, 70, 80, 90, 96, or 100 hours of patient sample receipt at a testing facility. In an aspect, the reporting of the CDM test results to the health care professional (e.g., doctor, nurse, or caregiver) is via fax, a website portal, email, or an EMR interface.

In an aspect, results of a CDM test are returned to a patient or a healthcare provider within 1, 2, 3, 4, 5, 6, 7, 8, 9, 10, 11, 12, 13, 14, or 15 days of patient sample receipt at a testing facility.

In an aspect, an intervention based on the results of a CDM test is determined 1 day, 2 days, 3 days, 4 days, 5 days, 6 days, 7 days, 8 days, 9 days, 10 days, 11 days, 12 days, 13 days, 14 days, 15 days, 20 days, 25 days, 1 month, 2 months, or 3 months after obtaining a sample from a patient.

As used herein, the term “improving a symptom” refers to reducing the severity of the one or more symptoms of a disease, disorder, or condition of a patient. In an aspect, blood pressure measurement is used as the assessment system for assessing changes in symptom severity before, during, or following one or more intervention or treatment steps. In an aspect, an improvement in one or more symptoms is assessed by measuring a patient's blood pressure and subsequently comparing the measured value to a clinical care guideline. In an aspect, a symptom is improved by improving or restoring blood pressure control. In an aspect, a symptom is improved by improving or restoring blood pressure control according to a guideline-based treatment target.

In an aspect, blood glucose measurement is used as the assessment system for assessing changes in symptom severity before, during, or following one or more intervention or treatment steps.

In an aspect, an improvement in one or more symptoms is assessed by measuring a patient's blood glucose and subsequently comparing the measured value to a clinical care guideline. In an aspect, a symptom is improved by improving or restoring glucose control. In an aspect, a symptom is improved by improving or restoring glucose control according to a guideline-based treatment target.

In an aspect, a cardiac monitoring test (e.g. EKG) is used as the assessment system for assessing changes in symptom severity before, during, or following one or more intervention or treatment steps.

In an aspect, an improvement in one or more symptoms is assessed by monitoring a patient's cardiac activity and subsequently comparing the test results to a clinical care guideline.

In an aspect, a respiratory test (e.g. pulmonary function tests (PFTs)) is used as the assessment system for assessing changes in symptom severity before, during, or following one or more intervention or treatment steps. In an aspect, an improvement in one or more symptoms is assessed by monitoring a patient's respiration and subsequently comparing the test results to a clinical care guideline. In an aspect, other assessment systems can be used to evaluate changes in a patient's symptom severity before, during, or following one or more intervention or treatment steps.

In an aspect, cardiac risk (e.g. stroke or myocardial infarction (MI)) is lowered by improved blood pressure control after an intervention or treatment step. In an aspect, cardiac risk (e.g. stroke, MI, and sudden cardiac death) is lowered if a patient improves adherence to one or more drugs after an intervention or treatment step. In an aspect, cardiac risk (e.g. stroke, MI, and sudden cardiac death) is lowered if a patient improves adherence to one or more drugs in the SGLT2-inhibitor and/or beta-blocker substance classes after an intervention or treatment step. In an aspect, cardiac risk (e.g. stroke and MI) is lowered if a patient improves adherence to one or more drugs after an intervention or treatment step. In an aspect, cardiac risk (e.g. stroke and MI) is lowered if a patient improves adherence to one or more drugs in the statin, anticoagulants, and/or antiplatelet substance classes after an intervention or treatment step. In an aspect, cardiac risk (e.g. sudden cardiac death and atrial fibrillation) is lowered if a patient improves adherence to one or more drugs after an intervention or treatment step. In an aspect, cardiac risk (e.g. sudden cardiac death and atrial fibrillation) is lowered if a patient improves adherence to one or more drugs in the anti-arrhythmic and/or beta-blocker substance classes after an intervention or treatment step. In an aspect, one or more HF symptoms are improved if a patient improves adherence to one or more drugs after an intervention or treatment step.

In an aspect, one or more HF symptoms are improved if a patient improves adherence to one or more drugs in the diuretics (e.g. loop diuretic and thiazide diuretics) substance class after an intervention or treatment step. In an aspect, cardiac risk (e.g. HF) is lowered if a patient improves adherence to goal-directed HF therapies with outcome benefits. In an aspect, one or more COPD symptoms are improved and risk of exacerbations are decreased if a patient improves adherence to one or more drugs after an intervention or treatment step. In an aspect, one or more COPD symptoms are improved and risk of exacerbations are decreased if a patient improves adherence to one or more drugs in the corticosteroid (e.g. inhaled corticosteroid (ICS)) substance class and/or beta agonist substance classes after an intervention or treatment step.

In an aspect, a treatment or intervention results in a symptom improvement of at least 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 11%, 12%, 13%, 14%, 15%, 16%, 17%, 18%, 19%, 20%, 25%, 30%, 35%, 40%, 45%, 50%, 60%, 70%, 80%, or 90% based on blood pressure. In an aspect, a treatment or intervention results in a symptom improvement of at least 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 11%, 12%, 13%, 14%, 15%, 16%, 17%, 18%, 19%, 20%, 25%, 30%, 35%, 40%, 45%, 50%, 60%, 70%, 80%, or 90% based on blood glucose. In an aspect, a treatment or intervention results in a symptom improvement of at least 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 11%, 12%, 13%, 14%, 15%, 16%, 17%, 18%, 19%, 20%, 25%, 30%, 35%, 40%, 45%, 50%, 60%, 70%, 80%, or 90% based on results of a cardiac monitoring test. In an aspect, a treatment or intervention results in a symptom improvement of at least 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 11%, 12%, 13%, 14%, 15%, 16%, 17%, 18%, 19%, 20%, 25%, 30%, 35%, 40%, 45%, 50%, 60%, 70%, 80%, or 90% based on results of a respiratory test. In an aspect, a treatment or intervention results in a symptom improvement of at least 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 11%, 12%, 13%, 14%, 15%, 16%, 17%, 18%, 19%, 20%, 25%, 30%, 35%, 40%, 45%, 50%, 60%, 70%, 80%, or 90% based on an appropriate assessment system. In an aspect, a treatment or intervention results in a symptom improvement of at least 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 11%, 12%, 13%, 14%, 15%, 16%, 17%, 18%, 19%, 20%, 25%, 30%, 35%, 40%, 45%, 50%, 60%, 70%, 80%, or 90% based on a patient's self assessment. In an aspect, a treatment or intervention results in a symptom improvement of at least 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 11%, 12%, 13%, 14%, 15%, 16%, 17%, 18%, 19%, 20%, 25%, 30%, 35%, 40%, 45%, 50%, 60%, 70%, 80%, or 90% based on a caregiver's assessment. In an aspect, a treatment or intervention results in a symptom improvement of at least 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 11%, 12%, 13%, 14%, 15%, 16%, 17%, 18%, 19%, 20%, 25%, 30%, 35%, 40%, 45%, 50%, 60%, 70%, 80%, or 90% based on an assessment system supported by clinical guidelines. In an aspect, a percent improvement in a symptom can be scaled by a factor or a multiple based on values provided herein.

In an aspect, a treatment or intervention achieves at least 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, or 10% reduction in blood pressure in a patient. In an aspect, a treatment or intervention achieves at least 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, or 10% reduction in blood glucose in a patient.

In an aspect, a symptom improvement based on any assessment system is measured after administering the intervention or treatment for at least 1, 2, 3, 4, 5, 6, 7, 8, 9, 10, or more weeks and compared to one or more measurements made prior to the intervention or treatment. In an aspect, a symptom improvement based on any assessment system is measured after administering the intervention or treatment for at least 1, 2, 3, 4, 5, 6, 7, 8, 9, 10, or more months and compared to one or more measurements made prior to the intervention or treatment.

In an aspect, a treatment or intervention achieves between 1% and 5%, between 5% and 10%, between 10% and 15%, between 15% and 20%, between 20% and 30%, between 30% and 40%, or between 40% and 50% reduction in symptom severity after 8 or more weeks of treatment or intervention as compared to before initiating the treatment or intervention. In an aspect, a treatment or intervention achieves between 1% and 5%, between 5% and 10%, between 10% and 15%, between 15% and 20%, between 20% and 30%, between 30% and 40%, or between 40% and 50% reduction in symptom severity after 16 or more weeks of treatment or intervention as compared to before initiating the treatment or intervention.

Methods according to the present disclosure provide for, and include, detecting NA and the presence and severity of DDIs in a sample obtained from a patient with a chronic condition. In an aspect, measurements and analysis are determine by an CDM testing apparatus or system. In an aspect, an apparatus or system according to the present disclosure comprises a device configured to measure one or more analytes, a processor that may be configured to electronically couple to the device to receive the measured values, and a non-transitory computer readable medium that may be configured to electronically couple to the processor and may comprise instructions stored thereon that, when executed on the processor, may perform the steps of analyzing the patient sample for the analytes, identifying a list of drugs recently ingested by the patient based on the analyzing step, determining if there is NA and or DDI, and determining an intervention or treatment step that addresses the NA and or DDI.

In an aspect, an apparatus or system comprises a device configured to measure one or more analytes of one or more drug classes selected from the group consisting of antiarrhythmics, antidepressants and antipsychotics, antiemetics and gastric reflux therapies, antiepileptics; antihypertensives, antimicrobials and antivirals, antithrombotics, asthma/COPD, cardiovascular, chemotherapeutic agents, cognitive enhancement agents, DM, DM2, inhaled corticosteroids and beta agonists, NSAIDs, skeletal muscle relaxant, steroids, CKD, foods and supplements, and miscellaneous drugs.

In an aspect, an apparatus or system comprises a device configured to further measure one or more analytes of one or more drug classes selected from the group consisting of amphetamines, anti-Parkinson agents, benzodiazepines, opiates, opioids, and mixed agonists-antagonists.

In an aspect, an apparatus or system comprises a device configured to measure between 10 and 50 analytes, between 50 and 100 analytes, between 100 and 150 analytes, between 150 and 200 analytes, between 200 and 250 analytes, or between 250 and 300 analytes. In an aspect, the apparatus or system comprises a device configured to measure analytes from between 1 and 5 drug classes, between 5 and 10 drug classes, between 10 and 15 drug classes, or between 15 and 20 drug classes.

In an aspect, an apparatus or system comprises a device configured to detect DDIs, and configured to measure one or more analytes of the antiarrhythmics drug class selected from the group consisting of amiodarone, desethylamiodarone, quinidine, ranolazine, and a combination thereof.

In an aspect, an apparatus or system comprises a device configured to detect DDIs, and configured to measure one or more analytes of the antidepressants and antipsychotic drug class selected from the group consisting of citalopram, N-desmethylcitalopram, duloxetine, fluvoxamine, fluoxetine, norfluoxetine, iloperidone, lurasidone, nefazodone, olanzapine, paroxetine, 7-hydroxy quetiapine, norquetiapine, quetiapine, 9-hydroxyrisperidone, norsertraline, sertraline, trazodone, O-desmethylvenlafaxine, venlafaxine, vortioxetine, and a combination thereof.

In an aspect, an apparatus or system comprises a device configured to detect DDIs, and configured to measure one or more analytes of the antiemetic and gastric reflux drug class selected from the group consisting of aprepitant, cimetidine, fosamprenavir, 4-hydroxy omeprazole sulfide, omeprazole sulfone, and a combination thereof.

In an aspect, an apparatus or system comprises a device configured to detect DDIs, and configured to measure one or more analytes of antiepileptics drug class selected from the group consisting of butalbital, carbamazepine, carbamazepine epoxide, phenobarbital, phenytoin, primidone, and a combination thereof.

In an aspect, an apparatus or system comprises a device configured to detect DDIs, and configured to measure one or more analytes of the antimicrobials and antivirals drug class selected from the group consisting of atazanavir, azithromycin, N-desmethyl azithromycin, ciprofloxacin, clarithromycin, cobicistat, darunavir, delavirdine, 8-hydroxyefavirenz, efavirenz, erythromycin, etravirine, fluconazole, amprenavir, indanavir, itraconazole, 2-hydroxyitraconazole, ketoconazole, levofloxacin, ofloxacin, metronidazole, 2-hydroxynelfinavir, nelfinavir, posaconazole, quinine, rifabutin, rifampin, rifapentine, hydroxy ritonavir, ritonavir, saquinavir, sulfamethoxazole, tipranavir, trimethoprim, voriconazole, voriconazole N-oxide, and a combination thereof.

In an aspect, an apparatus or system comprises a device configured to detect DDIs, and configured to measure one or more analytes of the cardiovascular drug class selected from the group consisting of amlodipine, amlodipine metabolite, apixaban, salicylic acid, chlorothiazide, clonidine, clopidogrel, clopidogrel COOH, digoxin, deacetyl diltiazem-N-oxide, diltiazem, fenofibrate, fenofibrate metabolite, gemfibrozil, hydrochlorothiazide, lovastatin, lovastatin acid, rivaroxaban, sacubitril, simvastatin, simvastatin acid, ticagrelor, triamterene, norverapamil, verapamil, 7-hydroxywarfarin, warfarin, and a combination thereof.

In an aspect, an apparatus or system comprises a device configured to detect DDIs, and configured to measure one or more analytes of the chemotherapeutic agents drug class selected from the group consisting of enzalutamide, nilotinib, pazopanib, and a combination thereof.

In an aspect, an apparatus or system comprises a device configured to detect DDIs, and configured to measure one or more analytes of the cognitive enhancement drug class selected from the group consisting of 3-hydroxyguanfacine, guanfacine, and a combination thereof.

In an aspect, an apparatus or system comprises a device configured to detect DDIs, and configured to measure one or more analytes of the DM/DM2 drug class selected from the group consisting of hydroxypioglitazone, pioglitazone, and a combination thereof.

In an aspect, apparatus or system comprises a device configured to detect DDIs, and configured to measure one or more analytes of the NSAIDs drug class selected from the group consisting of celecoxib, diclofenac, ibuprofen, indomethacin, ketorolac, meloxicam, naproxen, and a combination thereof.

In an aspect, an apparatus or system comprises a device configured to detect DDIs, and configured to measure one or more analytes of the skeletal muscle relaxant drug class selected from the group consisting of cyclobenzaprine, norcyclobenzaprine, dehydrotizanidine, tizanidine, and combination thereof.

In an aspect, an apparatus or system comprises a device configured to detect DDIs, and configured to measure one or more analytes of the foods and supplements drug class selected from the group consisting of bergaptol, dihydroxybergamottin, dihydrokavain, hyperforin, and a combination thereof.

In an aspect, an apparatus or system comprises a device configured to detect DDIs, and configured to measure one or more analytes of the miscellaneous drug class selected from the group consisting of avanafil, colchicine, cyclosporine, 7-hydroxymethotrexate, methotrexate, tacrolimus, and a combination thereof.

In an aspect, an apparatus or system comprises a device configured to detect NA, and configured to measure one or more analytes of the asthma/chronic obstructive pulmonary disease (COPD) drug class selected from the group consisting of Aclidinium, Albuterol/Levalbuterol, Arformoterol/Formoterol, Indacaterol, Olodaterol, Salmeterol, Vilanterol, Beclomethasone Monopropionate, Beclomethasone, Budesonide, Fluticasone Furoate, Fluticasone Propionate, Methylprednisolone, Roflumilast Metabolite, Roflumilast, Ipratropium, Tiotropium, Umeclidinium, and a combination thereof.

In an aspect, an apparatus or system comprises a device configured to detect NA, and configured to measure one or more analytes of the cardiovascular drug class selected from the group consisting of Hydralazine Metabolite, Acebutolol, Amlodipine Metabolite, Amlodipine, Apixaban, Salicylic Acid, Atenolol, Atorvastatin Lactone, Atorvastatin, Azilsartan, Benazepril, Benazeprilat, Bendroflumethiazide, Betaxolol, Bisoprolol, Bumetanide, Candesartan, Captopril, Carvedilol, Chlorothiazide, Chlorthalidone, Clopidogrel COOH, Clopidogrel, Dabigatran, Deacetyl Diltiazem-N-Oxide, Diltiazem, Dipyridamole, Enalapril, Enalaprilat, Eplerenone, Eprosartan, Dehydro Felodipine, Felodipine, Fluvastatin, Fosinopril, Fosinoprilat, Furosemide, Hydrochlorothiazide, Indapamide, Irbesartan, Isradipine, Labetalol, Lisinopril, Losartan Metabolite, Losartan, Lovastatin acid, Lovastatin, Methyclothiazide, Metolazone, alpha-Hydroxymetoprolol, Metoprolol, Moexipril, Moexiprilat, Nadolol, Nebivolol, Nicardipine, Nifedipine Carboxylate, Nifedipine, Nisoldipine, Olmesartan, Perindopril, Pindolol, Pitavastatin, Pravastatin, Propranolol, Quinapril, Quinaprilat, Ramipril Metabolite, Ramipril, Rivaroxaban, Rosuvastatin, Sacubitril Metabolite, Sacubitril, Simvastatin, Simvastatin Acid, Canrenoic Acid, Canrenone, Spironolactone, Telmisartan, Ticagrelor, Timolol, Torsemide, Trandolapril, Trandolaprilat, Valsartan, Norverapamil, Verapamil, 7-Hydroxywarfarin, Warfarin, and a combination thereof.

In an aspect, an apparatus or system comprises a device configured to detect NA, and configured to measure one or more analytes of the DM/DM2 drug class selected from the group consisting of Metformin (Glucophage), Linagliptin, Sitagliptin, Canagliflozin, Dapagliflozin, Empagliflozin, Glimepiride, Glipizide, Glyburide Metabolite, Glyburide, Hydroxypioglitazone, Pioglitazone, and a combination thereof.

A variety of further modifications and improvements in and to the methods of the present disclosure will be apparent to those skilled in the art. The following non-limiting embodiments are specifically envisioned:

1. A method for treating a chronic disease in a patient in need thereof, comprising:
obtaining an oral fluid sample from the patient;
analyzing the oral fluid for analytes of one or more drug classes selected from the group consisting of antiarrhythmics; antidepressants and antipsychotics; antiemetics and gastric reflux therapies; antiepileptics; antimicrobials and antivirals; cardiovascular; chemotherapeutic agents; cognitive enhancement agents; diabetes mellitus (DM); diabetes mellitus type 2 (DM2); nonsteroidal anti-inflammatory drugs (NSAIDs); skeletal muscle relaxant; foods and supplements; and miscellaneous drugs;
identifying a list of drugs recently ingested by the patient based on the analyzing step;
determining whether any drug-drug interaction (DDI) is present between the drugs in the list; and
if DDI is present, presenting a first treatment plan to the patient; or
if DDI is not present, presenting a second treatment plan to the patient.

2. The method of embodiment 1, wherein the list of drugs comprises prescription drugs, non-prescription drugs, and other miscellaneous substances.

3. The method of embodiment 1, further comprising administering an alternative intervention to the patient, wherein the alternative intervention does not interact with the remaining drugs in the list.

4. The method of embodiment 3, wherein the alternative intervention comprises administration of one or more new drugs to the patient.

5. The method of embodiment 1, further comprising administering one or more of the remaining drugs in the list to the patient at an increased dosage.

6. The method of embodiment 1, wherein the first treatment plan comprises one or more treatment options selected from the group consisting of implementing a monitoring plan for the patient, decreasing the dosage of one or more drugs determined as interacting with another drug in the list, increasing the dosage of one or more drugs determined as interacting with another drug in the list, discontinuing administration of one or more drugs determined as interacting with another drug in the list, continuing administration of the remaining drugs in the list, and a combination thereof.

7. The method of embodiment 1, wherein the second treatment plan comprises one or more treatment options selected from the group consisting of implementing one or more assessments for the presenting signs and symptoms experienced by the patient, continuing administration of the list of drugs, and a combination thereof.

8. The method of embodiment 1, wherein the analyzing step is performed by mass spectrometry.

9. The method of embodiment 8, wherein the mass spectrometry is high-performance liquid chromatography-tandem mass spectrometry (LC-MS/MS).

10. The method of embodiment 1, wherein the analytes of the antiarrhythmics drug class are selected from the group consisting of amiodarone, desethylamiodarone, quinidine, ranolazine, and a combination thereof.

11. The method of embodiment 1, wherein the analytes of the antidepressants and antipsychotic drug class are selected from the group consisting of citalopram, N-desmethylcitalopram, duloxetine, fluvoxamine, fluoxetine, norfluoxetine, iloperidone, lurasidone, nefazodone, olanzapine, paroxetine, 7-hydroxy quetiapine, norquetiapine, quetiapine, 9-hydroxyrisperidone, norsertraline, sertraline, trazodone, O-desmethylvenlafaxine, venlafaxine, vortioxetine, and a combination thereof.

12. The method of embodiment 1, wherein the analytes of the antiemetic and gastric reflux drug class are selected from the group consisting of aprepitant, cimetidine, fosamprenavir, 4-hydroxy omeprazole sulfide, omeprazole sulfone, and a combination thereof.

13. The method of embodiment 1, wherein the analytes of the antiepileptics drug class are selected from the group consisting of butalbital, carbamazepine, carbamazepine epoxide, phenobarbital, phenytoin, primidone, and a combination thereof.

14. The method of embodiment 1, wherein the analytes of the antimicrobials and antivirals drug class are selected from the group consisting of atazanavir, azithromycin, N-desmethyl azithromycin, ciprofloxacin, clarithromycin, cobicistat, darunavir, delavirdine, 8-hydroxyefavirenz, efavirenz, erythromycin, etravirine, fluconazole, amprenavir, indanavir, itraconazole, 2-hydroxyitraconazole, ketoconazole, levofloxacin, ofloxacin, metronidazole, 2-hydroxynelfinavir, nelfinavir, posaconazole, quinine, rifabutin, rifampin, rifapentine, hydroxy ritonavir, ritonavir, saquinavir, sulfamethoxazole, tipranavir, trimethoprim, voriconazole, voriconazole N-oxide, and a combination thereof.

15. The method of embodiment 1, wherein the analytes of the cardiovascular drug class are selected from the group consisting of amlodipine, amlodipine metabolite, apixaban, salicylic acid, chlorothiazide, clonidine, clopidogrel, clopidogrel COOH, digoxin, deacetyl diltiazem-N-oxide, diltiazem, fenofibrate, fenofibrate metabolite, gemfibrozil, hydrochlorothiazide, lovastatin, lovastatin acid, rivaroxaban, sacubitril, simvastatin, simvastatin acid, ticagrelor, triamterene, norverapamil, verapamil, 7-hydroxywarfarin, warfarin, and a combination thereof.

16. The method of embodiment 1, wherein the analytes of the chemotherapeutic agents drug class are selected from the group consisting of enzalutamide, nilotinib, pazopanib, and a combination thereof.

17. The method of embodiment 1, wherein the analytes of the cognitive enhancement drug class are selected from the group consisting of 3-hydroxyguanfacine, guanfacine, and a combination thereof.

18. The method of embodiment 1, wherein the analytes of the diabetes mellitus (DM)/diabetes mellitus type 2 (DM2) drug class are selected from the group consisting of hydroxypioglitazone, pioglitazone, and a combination thereof.

19. The method of embodiment 1, wherein the analytes of the NSAIDs drug class are selected from the group consisting of celecoxib, diclofenac, ibuprofen, indomethacin, ketorolac, meloxicam, naproxen, and a combination thereof.

20. The method of embodiment 1, wherein the analytes of the skeletal muscle relaxant drug class are selected from the group consisting of cyclobenzaprine, norcyclobenzaprine, dehydrotizanidine, tizanidine, and combination thereof.

21. The method of embodiment 1, wherein the analytes of the foods and supplements drug class are selected from the group consisting of bergaptol, dihydroxybergamottin, dihydrokavain, hyperforin, and a combination thereof.

22. The method of embodiment 1, wherein the analytes of the miscellaneous drug class are selected from the group consisting of avanafil, colchicine, cyclosporine, 7-hydroxymethotrexate, methotrexate, tacrolimus, and a combination thereof.

23. The method of embodiment 1, wherein the chronic disease is selected from the group consisting of chronic kidney disease (CKD), coronary heart disease (CHD), diabetes mellitus (DM), diabetes mellitus type 2 (DM2), chronic heart failure (CHF), stroke, coronary artery disease (CAD), hyperlipidemia, and hypertension.

24. The method of embodiment 1, wherein the patient was diagnosed with two or more indications selected from the group consisting of chronic kidney disease (CKD), coronary heart disease (CHD), diabetes mellitus (DM), diabetes mellitus type 2 (DM2), chronic heart failure (CHF), stroke, coronary artery disease (CAD), hyperlipidemia, and hypertension.

25. The method of embodiment 1, wherein about 1 milliliter of the oral fluid is obtained from the patient during the obtaining step.

26. The method of embodiment 1, wherein the oral fluid sample is obtained in a doctor's office.

27. The method of embodiment 1, wherein the oral fluid sample is shipped to a testing facility.

28. The method of embodiment 1, wherein the results of the analyzing, identifying, and determining steps are reported to the patient or a healthcare worker within 96 hours of oral fluid sample receipt at a testing facility.

29. The method of embodiment 28, wherein the results are reported via fax, a website portal, or an electronic health record (EMR) interface.

30. The method of embodiment 1, wherein prior to the treatment, the patient is administered 5 or more drugs concurrently.

31. A method for improving a symptom of a chronic disease in a patient in need thereof, comprising:
obtaining an oral fluid sample from the patient;
analyzing the oral fluid for analytes of one or more drug classes selected from the group consisting of antiarrhythmics; antidepressants and antipsychotics; antiemetics and gastric reflux therapies; antiepileptics; antimicrobials and antivirals; cardiovascular; chemotherapeutic agents; cognitive enhancement agents; diabetes mellitus (DM); diabetes mellitus type 2 (DM2); nonsteroidal anti-inflammatory drugs (NSAIDs); skeletal muscle relaxant; foods and supplements; and miscellaneous drugs;
identifying a list of drugs recently ingested by the patient based on the analyzing step;
determining that a drug-drug interaction (DDI) is present between the drugs in the list; and
determining an intervention, wherein the intervention comprises decreasing or increasing the dosage or discontinuing administration of one or more drugs determined as interacting with another drug in the list, and continuing administration of the remaining drugs in the list to the patient;
wherein the symptom is improved after the intervention in comparison to prior to the intervention.

32. The method of embodiment 31, wherein the list of drugs comprises prescription drugs, non-prescription drugs, and other miscellaneous substances.

33. The method of embodiment 31, wherein the symptom is improved by restoring blood pressure control according to a guideline-based treatment target.

34. The method of embodiment 31, wherein the symptom is improved by restoring glucose control according to a guideline-based treatment target.

35. The method of embodiment 31, wherein the symptom is improved based on results of one or more cardiac monitoring tests.

36. The method of embodiment 31, wherein the symptom is improved based on results of one or more respiratory tests.

37. The method of embodiment 31, further comprising administering an alternative intervention to the patient, wherein the alternative intervention does not interact with the remaining drugs in the list.

38. The method of embodiment 37, wherein the alternative intervention comprises administration of one or more new drugs to the patient.

39. The method of embodiment 31, further comprising administering one or more of the remaining drugs in the list to the patient at an increased dosage.

40. The method of embodiment 31, wherein the analyzing step is performed by mass spectrometry.

41. The method of embodiment 40, wherein the mass spectrometry is high-performance liquid chromatography-tandem mass spectrometry (LC-MS/MS).

42. The method of embodiment 31, wherein the analytes of the antiarrhythmics drug class are selected from the group consisting of amiodarone, desethylamiodarone, quinidine, ranolazine, and a combination thereof.

43. The method of embodiment 31, wherein the analytes of the antidepressants and antipsychotic drug class are selected from the group consisting of citalopram, N-desmethylcitalopram, duloxetine, fluvoxamine, fluoxetine, norfluoxetine, iloperidone, lurasidone, nefazodone, olanzapine, paroxetine, 7-hydroxy quetiapine, norquetiapine, quetiapine, 9-hydroxyrisperidone, norsertraline, sertraline, trazodone, O-desmethylvenlafaxine, venlafaxine, vortioxetine, and a combination thereof.

44. The method of embodiment 31, wherein the analytes of the antiemetic and gastric reflux drug class are selected from the group consisting of aprepitant, cimetidine, fosamprenavir, 4-hydroxy omeprazole sulfide, omeprazole sulfone, and a combination thereof.

45. The method of embodiment 31, wherein the analytes of the antiepileptics drug class are selected from the group consisting of butalbital, carbamazepine, carbamazepine epoxide, phenobarbital, phenytoin, primidone, and a combination thereof.

46. The method of embodiment 31, wherein the analytes of the antimicrobials and antivirals drug class are selected from the group consisting of atazanavir, azithromycin, N-desmethyl azithromycin, ciprofloxacin, clarithromycin, cobicistat, darunavir, delavirdine, 8-hydroxyefavirenz, efavirenz, erythromycin, etravirine, fluconazole, amprenavir, indanavir, itraconazole, 2-hydroxyitraconazole, ketoconazole, levofloxacin, ofloxacin, metronidazole, 2-hydroxynelfinavir, nelfinavir, posaconazole, quinine, rifabutin, rifampin, rifapentine, hydroxy ritonavir, ritonavir, saquinavir, sulfamethoxazole, tipranavir, trimethoprim, voriconazole, voriconazole N-oxide, and a combination thereof.

47. The method of embodiment 31, wherein the analytes of the cardiovascular drug class are selected from the group consisting of amlodipine, amlodipine metabolite, apixaban, salicylic acid, chlorothiazide, clonidine, clopidogrel, clopidogrel COOH, digoxin, deacetyl diltiazem-N-oxide, diltiazem, fenofibrate, fenofibrate metabolite, gemfibrozil, hydrochlorothiazide, lovastatin, lovastatin acid, rivaroxaban, sacubitril, simvastatin, simvastatin acid, ticagrelor, triamterene, norverapamil, verapamil (calan), 7-hydroxywarfarin, warfarin, and a combination thereof.

48. The method of embodiment 31, wherein the analytes of the chemotherapeutic agents drug class are selected from the group consisting of enzalutamide, nilotinib, pazopanib, and a combination thereof.

49. The method of embodiment 31, wherein the analytes of the cognitive enhancement drug class are selected from the group consisting of 3-hydroxyguanfacine, guanfacine, and a combination thereof.

50. The method of embodiment 31, wherein the analytes of the diabetes mellitus (DM)/diabetes mellitus type 2 (DM2) drug class are selected from the group consisting of hydroxypioglitazone, pioglitazone, and a combination thereof.

51. The method of embodiment 31, wherein the analytes of the NSAIDs drug class are selected from the group consisting of celecoxib, diclofenac, ibuprofen, indomethacin, ketorolac, meloxicam, naproxen, and a combination thereof.

52. The method of embodiment 31, wherein the analytes of the skeletal muscle relaxant drug class are selected from the group consisting of cyclobenzaprine, norcyclobenzaprine, dehydrotizanidine, tizanidine, and combination thereof.

53. The method of embodiment 31, wherein the analytes of the foods and supplements drug class are selected from the group consisting of bergaptol, dihydroxybergamottin, dihydrokavain, hyperforin, and a combination thereof.

54. The method of embodiment 31, wherein the analytes of the miscellaneous drug class are selected from the group consisting of avanafil, colchicine, cyclosporine, 7-hydroxymethotrexate, methotrexate, tacrolimus, and a combination thereof.

55. The method of embodiment 31, wherein the chronic disease is selected from the group consisting of chronic kidney disease (CKD), coronary heart disease (CHD), diabetes mellitus (DM), diabetes mellitus type 2 (DM2), chronic heart failure (CHF), stroke, coronary artery disease (CAD), hyperlipidemia, and hypertension.

56. The method of embodiment 31, wherein the patient was diagnosed with two or more indications selected from the group consisting of chronic kidney disease (CKD), coronary heart disease (CHD), diabetes mellitus (DM), diabetes mellitus type 2 (DM2), chronic heart failure (CHF), stroke, coronary artery disease (CAD), hyperlipidemia, and hypertension.

57. The method of embodiment 31, wherein about 1 milliliter of the oral fluid is obtained from the patient during the obtaining step.

58. The method of embodiment 31, wherein the oral fluid sample is obtained in a doctor's office.

59. The method of embodiment 31, wherein the oral fluid sample is shipped to a testing facility.

60. The method of embodiment 31, wherein the results of the analyzing, identifying, and determining steps are reported to the patient or a healthcare worker within 96 hours of oral fluid sample receipt at a testing facility.

61. The method of embodiment 60, wherein the results are reported via fax, a website portal, or an electronic health record (EMR) interface.

62. The method of embodiment 31, wherein prior to the improvement, the patient is administered 5 or more drugs concurrently.

63. A method for treating a chronic disease in a patient in need thereof, comprising:
obtaining an oral fluid sample from the patient;
analyzing the oral fluid for analytes of one or more drug classes selected from the group consisting of asthma/chronic obstructive pulmonary disease (COPD); cardiovascular; diabetes mellitus (DM); diabetes mellitus type 2 (DM2); and chronic kidney disease (CKD);
identifying a list of drugs recently ingested by the patient based on the analyzing step;
determining if there is medication nonadherence (NA) in the list of drugs by generating a list of drugs with NA and a list of drugs without NA; and
if NA is present, presenting a first treatment plan to the patient; or
if NA is not present, presenting a second treatment plan to the patient.

64. The method of embodiment 63, wherein the determining step comprises generating compliance calls based on comparing against a prescription drug list.

65. The method of embodiment 63, wherein the list of drugs comprises prescription drugs, non-prescription drugs, and other miscellaneous substances.

66. The method of embodiment 63, wherein the first treatment plan comprises one or more treatment options selected from the group consisting of implementing a monitoring plan for the patient, decreasing the dosage of one or more drugs with NA, increasing the dosage one or more drugs with NA, discontinuing administration of one or more drugs with NA, continuing administration of the list of drugs without NA, administering an increased dosage of one or more drugs in the list of drugs, advising the patient about the importance of adherence, providing aids and tools to increase adherence of drugs in the list of drugs with NA, and a combination thereof.

67. The method of embodiment 63, further comprising administering an alternative intervention to the patient, wherein the alternative intervention comprises administration of one or more new drugs to the patient.

68. The method of embodiment 63, further comprising administering one or more drugs with NA in a titrated dosage to the patient.

69. The method of embodiment 63, wherein the second treatment plan comprises one or more treatment options selected from the group consisting of implementing one or more assessments for the presenting signs and symptoms experienced by the patient, continuing administration of the list of drugs, and a combination thereof.

70. The method of embodiment 63, wherein the analyzing step is performed by mass spectrometry.

71. The method of embodiment 70, wherein the mass spectrometry is high-performance liquid chromatography-tandem mass spectrometry (LC-MS/MS).

72. The method of embodiment 63, wherein the analytes of the asthma/chronic obstructive pulmonary disease (COPD) drug class are selected from the group consisting of Aclidinium, Albuterol/Levalbuterol, Arformoterol/Formoterol, Indacaterol, Olodaterol, Salmeterol, Vilanterol, Beclomethasone Monopropionate, Beclomethasone, Budesonide, Fluticasone Furoate, Fluticasone Propionate, Methylprednisolone, Roflumilast Metabolite, Roflumilast, Ipratropium, Tiotropium, Umeclidinium, and a combination thereof.

73. The method of embodiment 63, wherein the analyte of the cardiovascular drug class is Hydralazine Metabolite.

74. The method of embodiment 63, wherein the analytes of the cardiovascular drug class are selected from the group consisting of Acebutolol, Amlodipine Metabolite, Amlodipine, Apixaban, Salicylic Acid, Atenolol, Atorvastatin Lactone, Atorvastatin, Azilsartan, Benazepril, Benazeprilat, Bendroflumethiazide, Betaxolol, Bisoprolol, Bumetanide, Candesartan, Captopril, Carvedilol, Chlorothiazide, Chlorthalidone, Clopidogrel COOH, Clopidogrel, Dabigatran, Deacetyl Diltiazem-N-Oxide, Diltiazem, Dipyridamole, Enalapril, Enalaprilat, Eplerenone, Eprosartan, Dehydro Felodipine, Felodipine, Fluvastatin, Fosinopril, Fosinoprilat, Furosemide, Hydrochlorothiazide, Indapamide, Irbesartan, Isradipine, Labetalol, Lisinopril, Losartan Metabolite, Losartan, Lovastatin acid, Lovastatin, Methyclothiazide, Metolazone, alpha-Hydroxymetoprolol, Metoprolol, Moexipril, Moexiprilat, Nadolol, Nebivolol, Nicardipine, Nifedipine Carboxylate, Nifedipine, Nisoldipine, Olmesartan, Perindopril, Pindolol, Pitavastatin, Pravastatin, Propranolol, Quinapril, Quinaprilat, Ramipril Metabolite, Ramipril, Rivaroxaban, Rosuvastatin, Sacubitril Metabolite, Sacubitril, Simvastatin, Simvastatin Acid, Canrenoic Acid, Canrenone, Spironolactone, Telmisartan, Ticagrelor, Timolol, Torsemide, Trandolapril, Trandolaprilat, Valsartan, Norverapamil, Verapamil, 7-Hydroxywarfarin, Warfarin, and a combination thereof.

75. The method of embodiment 63, wherein the analytes of the diabetes mellitus (DM)/diabetes mellitus type 2 (DM2) drug class are selected from the group consisting of Metformin (Glucophage), Linagliptin, Sitagliptin, Canagliflozin, Dapagliflozin, Empagliflozin, Glimepiride, Glipizide, Glyburide Metabolite, Glyburide, Hydroxypioglitazone, Pioglitazone, and a combination thereof.

76. The method of embodiment 63, wherein the chronic disease is selected from the group consisting of chronic kidney disease (CKD), coronary heart disease (CHD), diabetes mellitus (DM), diabetes mellitus type 2 (DM2), chronic heart failure (CHF), stroke, coronary artery disease (CAD), hyperlipidemia, and hypertension.

77. The method of embodiment 63, wherein the patient was diagnosed with two or more indications selected from the group consisting of chronic kidney disease (CKD), coronary heart disease (CHD), diabetes mellitus (DM), diabetes mellitus type 2 (DM2), chronic heart failure (CHF), stroke, coronary artery disease (CAD), hyperlipidemia, and hypertension.

78. The method of embodiment 63, wherein the wherein about 1 milliliter of the oral fluid is obtained from the patient during the obtaining step.

79. The method of embodiment 63, wherein the oral fluid sample is obtained in a doctor's office.

80. The method of embodiment 63, wherein the oral fluid sample is shipped to a testing facility.

81. The method of embodiment 63, wherein the results of the analyzing, identifying, and determining steps are reported to the patient or a healthcare worker within 96 hours of oral fluid sample receipt at a testing facility.

82. The method of embodiment 81, wherein the results are reported via fax, a website portal, or an electronic health record (EMR) interface.

83. The method of embodiment 63, wherein prior to the treatment, the patient is administered 5 or more drugs concurrently.

84. A method of improving a symptom of a chronic disease in a patient in need thereof, comprising:
obtaining an oral fluid sample from the patient;
analyzing the oral fluid for analytes of one or more drug classes selected from the group consisting of asthma/chronic obstructive pulmonary disease (COPD); cardiovascular; diabetes mellitus (DM); diabetes mellitus type 2 (DM2); and chronic kidney disease (CKD);
identifying a list of drugs recently ingested by the patient based on the analyzing step;
determining that there is medication nonadherence (NA) in the list of drugs by generating a list of drugs with NA and a list of drugs without NA; and
perform the step of:
decreasing or increasing the dosage or discontinuing administration of one or more drugs with NA, continuing administration of the list of drugs without NA to the patient;
wherein the symptom is improved after performing the step in comparison to prior to performing the step.

85. The method of embodiment 84, wherein the determining step comprises generating compliance calls based on comparing against a prescription drug list.

86. The method of embodiment 84, wherein the list of drugs comprises prescription drugs, non-prescription drugs, and other miscellaneous substances.

87. The method of embodiment 84, further comprising a step of advising the patient about the importance of adherence.

88. The method of embodiment 84, further comprising a step of providing aids and tools to increase adherence of drugs in the list of drugs with NA.

89. The method of embodiment 84, wherein the symptom is improved in the patient by restoring blood pressure control according to a guideline-based treatment target.

90. The method of embodiment 84, wherein the symptom is improved in the patient by restoring blood pressure levels similar to patients who have persistently maintained adherence to medication.

91. The method of embodiment 84, wherein the symptom is improved by restoring glucose control according to a guideline-based treatment target.

92. The method of embodiment 84, wherein the symptom is improved based on results of one or more cardiac monitoring tests.

93. The method of embodiment 84, wherein the symptom is improved based on results of one or more respiratory tests.

94. The method of embodiment 84, further comprising administering an alternative intervention to the patient, wherein the alternative intervention comprises administration of one or more new drugs to the patient.

95. The method of embodiment 84, further comprising administering an increased dosage of one or more drugs in the list of drugs to the patient.

96. The method of embodiment 84, wherein the analyzing step is performed by mass spectrometry.

97. The method of embodiment 96, wherein the mass spectrometry is high-performance liquid chromatography-tandem mass spectrometry (LC-MS/MS).

98. The method of embodiment 84, wherein the analytes of the asthma/chronic obstructive pulmonary disease (COPD) drug class are selected from the group consisting of Aclidinium, Albuterol/Levalbuterol, Arformoterol/Formoterol, Indacaterol, Olodaterol, Salmeterol, Vilanterol, Beclomethasone Monopropionate, Beclomethasone, Budesonide, Fluticasone Furoate, Fluticasone Propionate, Methylprednisolone, Roflumilast Metabolite, Roflumilast, Ipratropium, Tiotropium, Umeclidinium, and a combination thereof.

99. The method of embodiment 84, wherein the analyte of the cardiovascular drug class is Hydralazine Metabolite.

100. The method of embodiment 84, wherein the analytes of the cardiovascular drug class is Acebutolol, Amlodipine Metabolite, Amlodipine, Apixaban, Salicylic Acid, Atenolol, Atorvastatin Lactone, Atorvastatin, Azilsartan, Benazepril, Benazeprilat, Bendroflumethiazide, Betaxolol, Bisoprolol, Bumetanide, Candesartan, Captopril, Carvedilol, Chlorothiazide, Chlorthalidone, Clopidogrel COOH, Clopidogrel, Dabigatran, Deacetyl Diltiazem-N-Oxide, Diltiazem, Dipyridamole, Enalapril, Enalaprilat, Eplerenone, Eprosartan, Dehydro Felodipine, Felodipine, Fluvastatin, Fosinopril, Fosinoprilat, Furosemide, Hydrochlorothiazide, Indapamide, Irbesartan, Isradipine, Labetalol, Lisinopril, Losartan Metabolite, Losartan, Lovastatin acid, Lovastatin, Methyclothiazide, Metolazone, alpha-Hydroxymetoprolol, Metoprolol, Moexipril, Moexiprilat, Nadolol, Nebivolol, Nicardipine, Nifedipine Carboxylate, Nifedipine, Nisoldipine, Olmesartan, Perindopril, Pindolol, Pitavastatin, Pravastatin, Propranolol, Quinapril, Quinaprilat, Ramipril Metabolite, Ramipril, Rivaroxaban, Rosuvastatin, Sacubitril Metabolite, Sacubitril, Simvastatin, Simvastatin Acid, Canrenoic Acid, Canrenone, Spironolactone, Telmisartan, Ticagrelor, Timolol, Torsemide, Trandolapril, Trandolaprilat, Valsartan, Norverapamil, Verapamil, 7-Hydroxywarfarin, Warfarin, and a combination thereof.

101. The method of embodiment 84, wherein the analytes of the diabetes mellitus (DM)/diabetes mellitus type 2 (DM2) drug class are selected from the group consisting of Metformin (Glucophage), Linagliptin, Sitagliptin, Canagliflozin, Dapagliflozin, Empagliflozin, Glimepiride, Glipizide, Glyburide Metabolite, Glyburide, Hydroxypioglitazone, Pioglitazone, and a combination thereof.

102. The method of embodiment 84, wherein the chronic disease is selected from the group consisting of chronic kidney disease (CKD), coronary heart disease (CHD), diabetes mellitus (DM), diabetes mellitus type 2 (DM2), chronic heart failure (CHF), stroke, coronary artery disease (CAD), hyperlipidemia, and hypertension.

103. The method of embodiment 84, wherein the patient was diagnosed with two or more indications selected from the group consisting of chronic kidney disease (CKD), coronary heart disease (CHD), diabetes mellitus (DM), diabetes mellitus type 2 (DM2), chronic heart failure (CHF), stroke, coronary artery disease (CAD), hyperlipidemia, and hypertension.

104. The method of embodiment 84, wherein the wherein about 1 milliliter of the oral fluid is obtained from the patient during the obtaining step.

105. The method of embodiment 84, wherein the oral fluid sample is obtained in a doctor's office.

106. The method of embodiment 84, wherein the oral fluid sample is shipped to a testing facility.

107. The method of embodiment 84, wherein the results of the analyzing, identifying, and determining steps are reported to the patient or a healthcare worker within 96 hours of oral fluid sample receipt at a testing facility.

108. The method of embodiment 107, wherein the results are reported via fax, a website portal, or an electronic health record (EMR) interface.

109. The method of embodiment 84, wherein prior to the treatment, the patient is administered 5 or more drugs concurrently.

110. A method for improving management of a chronic disease in a patient in need thereof, comprising the steps of:
i. obtaining an oral fluid sample from the patient;
ii. analyzing the oral fluid for analytes of one or more drug classes;
iii. determining whether a drug-drug interaction (DDI) is present based on step ii;
iv. determining an intervention based on the results from steps ii-iii,
   wherein if DDI is present, the intervention comprises:
      a. counseling the patient on DDI;
      b. documenting DDI in the patient's chart;
      c. documenting a newly identified adverse drug effect and/or therapeutic non-response in the patient's chart;
      d. adjusting or discontinuing a medication that is causing DDI;
      e. utilizing a new or maximizing an existing non-pharmacologic treatment option; and
      f. using a monitoring plan for potential increased symptoms of DDI;
   wherein if DDI is not present, the intervention comprises:
      g. counseling the patient on disease progression or other underlying issues related to adverse drug reaction (ADR);
      h. maintaining or adjusting the patient's current medication regimen; and
      i. using a monitoring plan for potential increased symptoms attributed to disease progression or other underlying issues related to ADR; and
v. presenting the intervention to the patient;
   wherein the management of the chronic disease is improved after the intervention in comparison to prior to the intervention.

111. The method of embodiment 110, wherein the method comprises a further step vi of implementing the intervention.

112. The method of embodiment 110, wherein step iv is performed at a later time point after step i, wherein the later time point is selected from the group consisting of 5 days, 10 days, 15 days, 20 days, 1 month, 2 months, and 3 months.

113. The method of embodiment 110, wherein during step iii, if DDI is present, the one or more DDIs is each further classified into categories comprising moderate DDI or severe DDI.

114. The method of embodiment 110, wherein the improvement is assessed based on results obtained from performing steps i-iii a second time at a later time point after the intervention.

115. The method of embodiment 114, wherein a decrease in the number of DDIs determined from the second time performing step iii compared to the first time performing step iii is indicative of improvement in management of the chronic disease in the patient.

116. The method of embodiment 114, wherein if the same DDI is present as determined from the second time performing step iii compared to the first time performing step iii, a change in classification of the same DDI to a lower severity the second time performing step iii compared to the first time performing step iii is indicative of improvement in management of the chronic disease in the patient.

117. The method of embodiment 114, wherein the later time point is selected from the group consisting of 1 month, 2 months, 3 months, 4 months, 5 months, 6 months, 7 months, 8 months, 9 months, 10 months, 11 months, and 12 months.

118. The method of embodiment 110, wherein the results of steps ii and iii are returned to the patient or a healthcare provider within 5-10 calendar days of the oral fluid sample receipt at a testing facility.

119. The method of embodiment 110, wherein the one or more drug classes is selected from the group consisting of antiarrhythmics; antidepressants and antipsychotics; antiemetics and gastric reflux therapies; antiepileptics; antimicrobials and antivirals; cardiovascular; chemotherapeutic agents; cognitive enhancement agents; diabetes mellitus (DM); diabetes mellitus type 2 (DM2); nonsteroidal anti-inflammatory drugs (NSAIDs); skeletal muscle relaxant; foods and supplements; and miscellaneous drugs.

120. The method of embodiment 110, wherein the patient is administered 5 or more medications concurrently.

121. The method of embodiment 110, wherein the chronic disease is selected from the group consisting of chronic kidney disease (CKD), coronary heart disease (CHD), diabetes mellitus (DM), diabetes mellitus type 2 (DM2), chronic heart failure (CHF), stroke, coronary artery disease (CAD), hyperlipidemia, and hypertension.

122. The method of embodiment 110, wherein step ii is performed using high-performance liquid chromatography-tandem mass spectrometry (LC-MS/MS).

123. A method for improving management of a chronic disease in a patient in need thereof, comprising the steps of:
i. obtaining an oral fluid sample from the patient;
ii. analyzing the oral fluid for analytes of one or more drug classes;
iii. determining whether one or more medication nonadherence (NA) is present based on step ii, wherein each of the one or more NA corresponds to an analyte abnormality;
iv. determining an intervention based on the results from steps ii-iii,
   wherein if NA is present, the intervention comprises:
      a. counseling the patient on NA;
      b. documenting NA on the patient's chart;
      c. continuing, adjusting, or discontinuing a medication that is causing NA;
      d. utilizing a tool to mitigate NA; and
      e. treating a secondary condition that may be the potential cause of NA;
   wherein if NA is not present, the intervention comprises:
      f. counseling the patient on disease progression or other underlying issues related to adverse drug reaction (ADR);
      g. maintaining or adjusting the patient's current medication regimen;
      h. using a monitoring plan for potential increased symptoms attributed to disease progression or other underlying issues related to ADR; and
v. presenting the intervention to the patient;
   wherein the management of the chronic disease is improved after the intervention in comparison to prior to the intervention.

124. The method of embodiment 123, wherein the method comprises a further step vi of implementing the intervention.

125. The method of embodiment 123, wherein step iv is performed at a later time point after step i, wherein the later time point is selected from the group consisting of 5 days, 10 days, 15 days, 20 days, 1 month, 2 months, and 3 months.

126. The method of embodiment 123, wherein the improvement is assessed based on results obtained from performing steps i-iii a second time at a later time point after the intervention.

127. The method of embodiment 126, wherein a decrease in the number of NAs determined from the second time performing step iii compared to the first time performing step iii is indicative of improvement in management of the chronic disease in the patient.

128. The method of embodiment 126, wherein the later time point is selected from the group consisting of 1 month, 2 months, 3 months, 4 months, 5 months, 6 months, 7 months, 8 months, 9 months, 10 months, 11 months, and 12 months.

129. The method of embodiment 123, wherein the results of steps ii and iii are returned to the patient or a healthcare provider within 5-10 calendar days of the oral fluid sample receipt at a testing facility.

130. The method of embodiment 123, wherein the tool to mitigate NA is selected from the group consisting of smart reminder, alarm setting, and digital pill.

131. The method of embodiment 123, wherein the one or more drug classes is selected from the group consisting of asthma/chronic obstructive pulmonary disease (COPD); cardiovascular; diabetes mellitus (DM); diabetes mellitus type 2 (DM2); and chronic kidney disease (CKD).

132. The method of embodiment 123, wherein the patient is administered 5 or more medications concurrently.

133. The method of embodiment 123, wherein the chronic disease is selected from the group consisting of chronic kidney disease (CKD), coronary heart disease (CHD), diabetes mellitus (DM), diabetes mellitus type 2 (DM2), chronic heart failure (CHF), stroke, coronary artery disease (CAD), hyperlipidemia, and hypertension.

134. The method of embodiment 123, wherein step ii is performed using high-performance liquid chromatography-tandem mass spectrometry (LC-MS/MS).

135. The method of embodiment 123, wherein the secondary condition that may be the potential cause of NA is depression.

136. The method of embodiment 123, wherein the analyte abnormality is classified into categories comprising: A. prescribed to the patient but not detected in step ii; or B. detected in step ii but not prescribed to the patient.

### EXAMPLES

The following examples are put forth so as to provide those of ordinary skill in the art with a complete disclosure and description of how to make and use the present invention, and are not intended to limit the scope of what the inventors regard as their invention, nor are they intended to represent or imply that the experiments below are all of or the only experiments performed. It will be appreciated by persons skilled in the art that numerous variations and/or modifications may be made to the invention as shown in the specific aspects without departing from the spirit or scope of the invention as broadly described. The present aspects are, therefore, to be considered in all respects as illustrative and not restrictive.

EXAMPLE 1: A study to evaluate a novel CDM test that uses an oral sample from a patient to detect NA and the presence and severity of DDIs. The purpose of the study is to determine the clinical utility of the CDM test.

### Study Methods

### Study Overview

A randomized controlled cross-sectional study is conducted using simulated Clinical Performance and Value® (CPV®) vignettes. The study assesses the clinical utility of the CDM test among US primary care physicians in the evaluation, work-up, diagnosis, and management of patients with chronic cardiometabolic disease with either NA, DDIs, or disease progression. The participating physicians' clinical care is measured before and after introduction of the CDM test. The participating physicians each cares for three simulated CPV® patients per two rounds, for a total of six patients.

### Study Ethics

This study is conducted in accordance with ethical standards, approved by the Advarra Institutional Review Board, Columbia, MD, USA, and is listed in clinicaltrials.gov (NCT05192590). Informed consent is obtained from all participants.

### Physician Selection

American practicing primary care physicians (PCP) are recruited and enrolled from a national roster. The eligibility criteria are whether: 1) they are board-certified and currently practicing as a family medicine or internal medicine physician, 2) they have been practicing as a board-certified physician in internal or family medicine between 2-35 years, 3) they practice in a community or non-academic setting, 4) they care for more than 40 patients weekly, 5) they commonly treat patients with cardiometabolic conditions such as atrial fibrillation, coronary artery disease, heart failure (HF), diabetes mellitus (DM), hypertension (HTN), and hyperlipidemia, 6) they practice in the U.S., 7) they speak English, 8) they have internet access, and 9) they provide informed and voluntary consent into the study.

261 PCPs meet the inclusion criteria. Of this total, 8 participants are withdrawn from the study due to retracted consent, and 17 participants do not complete all six of their cases. A total of 699 simulated patient cases are completed by a total of 233 physicians. Of these 233 physicians, 78 are in the control group, 78 are in the intervention 1 group, and 77 are in the intervention 2 group. In the first round of cases, all physicians are naïve to the CDM test and do not have access to the CDM test results.

### Intervention

Identical educational materials on the CDM test is provided to the two intervention groups. The educational materials consist of a physician-targeted slide deck, a fact sheet which detailed the science behind the test, the sample collection method, and a sample report. Both intervention groups are required to view these materials before progressing to round 2 and completing the study. Two weeks after reviewing the educational materials, the intervention group physicians are asked to complete three new CPV patients during round 2 of the study.

The two intervention groups consist of intervention 1, wherein the participants are given the CDM test results as they complete the round 2 cases, and intervention 2, whose participants receive the same education materials but had the option of ordering the CDM test while they care for the round 2 simulated patients. Control arm physicians in round 2 have access to the current standard of care diagnostic tools but do not have access to the CDM test results.

### Data Sources

Two sources of data are used for this study: the physician questionnaire which query about the demographic background, physician characteristics and their practice characteristics, and the physicians' responses to the simulated patients, or CPVs.

### Physician Survey

After physicians are enrolled into the study, they are asked to answer a brief questionnaire detailing their practice, their patient level and types, and their own demographic background. The survey include questions on employment status, location of practice, practice type, and patient make-up, among other.

### Clinical Performance and Value (CPV) Vignettes

CPVs are a validated online patient simulation tool owned by QURE Healthcare which have been widely used to measure clinical care. Several inventors, through their affiliation with QURE Healthcare, are authorized to use the CPV vignettes. These vignettes are open-ended questions simulating typical clinical encounters involving four domains of care: 1) history taking, 2) physical examination, 3) diagnostic workup, 4) making a diagnosis and treatment (DxTx) plan including follow-on care.

Between 61 and 74 evidence-based criteria are evaluated for each CPV. Participant responses are scored by two trained expert physicians, working independently, using pre-determined criteria (see Table 1) based on current standards of care to measure individual physician care. In cases of disagreement, a third physician is to adjudicate for the final score. All three physicians are blinded to the study arm assignment of the participant. A quality-of-care percentage score is generated based upon the number of responses matching the evidence-based criteria (range from 0% to 100%). Higher percentage scores indicate greater adherence to the evidence-base criteria in the clinical care provided.

TABLE 1
Pre-determined Case Scoring Criteria.

| | |
|---|---|
| HISTORY | Onset and duration of symptoms |
| DOMAIN | Nature, progression and severity of symptoms |
| | Associated symptoms |
| | Previous workup/laboratory tests/imaging studies |
| | related to the complaint |
| | Other medical problems and medical consults or |
| | hospitalizations |
| | Past surgeries |
| | Medications (prescription and over the counter) |
| | Allergies to medications |
| | Review of systems |
| | History of notable diseases in family members |
| | Alcohol, tobacco, illicit drug use |
| | Lifestyle: diet, stressors, and physical activity |
| | Social and economic determinants of health |
| | (employment status, insurance, living arrangement, |
| | relationship status) |
| PHYSICAL | General |
| EXAM | HEENT/Head/Eyes/Ears/Nose/Throat |
| DOMAIN | Neck |
| | Lungs/Respiratory/Chest/Pulmonary |
| | Cardiovascular/Cardiac/Heart/CV |
| | Abdomen/Gastrointestinal/GI |
| | Extremities/Musculoskeletal/MSK/Back |
| | Dermatologic/Skin |
| | Neurologic, as appropriate |
| | Psychiatric/Depression Screening, as appropriate |
| WORKUP | Relevant ancillary tests (blood tests, urine tests, etc.) |
| DOMAIN | Relevant imaging tests |
| | Other applicable diagnostic procedures |
| DIAGNOSIS | Primary clinical diagnosis |
| DOMAIN | Secondary diagnosis/-es |
| | Applicable to specific case type: Medication non- |
| | adherence/Drug-drug interaction |
| TREATMENT | Applicable to specific case type: Advise about |
| DOMAIN | importance of adherence, aids/tools to increase |
| | adherence, discontinue interacting medications, |
| | shift to different medication, increase medication dose, |
| | order interventions to address disease progression |
| | Supportive treatment |
| | Subspecialty referrals |
| | Non-pharmacologic management |
| | Follow-up office visit |

We note the DxTx score is helpful to understand the challenges clinicians face-DDI DxTx scores are 22.9% in a previous research study indicating that both diagnosis and treatment of DDIs are significant clinical problems (Peabody et al., “Drug-Drug Interaction Assessment and Identification in the Primary Care Setting,” Journal of Clinical Medicine Research 10(11):806 (2018)). CPV cases are used to evaluate and compare clinical practices of healthcare providers in a comprehensive range of clinical conditions and types of clinical practices.

### Chronic Cardiometabolic Disease Vignettes

Nine CPV vignettes are constructed on a 3×3 matrix with three patient case types and three variants. The case types include a patient with atrial fibrillation (Afib), a patient with heart failure (HF), and a patient with diabetes or hypertension (DM/HTN). The three variants include three patients who are not at their therapeutic goals because of NA but no DDI, referred to as NA patients; three patients who are not at their therapeutic goals (but adherent to medication) because of a DDI, referred to as the DDI patients; and three patients who are both adherent to their medications and have no DDI but not at their therapeutic goals because of disease progression, referred to as AND patients. For the AND patients, a diagnosis of disease progression is made with either explicitly diagnosing worsening disease, selecting a new medication, increasing the dose of medication, or referring to a specialist. To avoid any ordering effect, every participant cares for three CPV patients, one randomly assigned from each case type and from each variant type. The CPVs are typical patients with these three conditions and are summarized in Table 2.

TABLE 2
CPV case matrix.

| | | | Variant C: |
|---|---|---|---|
| | Variant A: | Variant B: | CDM Test shows no NA |
| | CDM Test shows NA but | CDM Test shows DDI but | and no DDI. |
| Case Types | no DDI. | not NA. | Disease Progression |
| Atrial | 77-year old woman | 81-year-old woman on | 66-year-old man on follow- |
| fibrillation | reporting depressive | follow-up after recent | up after recent |
| (Afib) | symptoms while on follow- | transient ischemic attack. | cardioembolic stroke. |
| | up for Afib (on warfarin), | Known non-valvular Afib | Blood pressure controlled. |
| | hypertension and | (on dabigatran), diabetes, | TEE showed LA thrombus |
| | dyslipidemia. Blood | dyslipidemia, and chronic | during stroke workup. |
| | pressure, heart rate are | low back pain. Recently | Known Afib (on apixaban), |
| | controlled. INR is slightly | started an OTC herbal | dyslipidemia, anxiety, and |
| | below target. | supplement to help | benign prostate |
| | | address symptoms of | enlargement. Recently |
| | | anxiety. | started taking an OTC |
| | | | supplement for stress |
| | | | alleviation. |
| Heart failure | 67-year-old man with heart | 69-year-old man with | 64-year-old woman with |
| (HF) | failure symptoms | known HF who presents | heart failure symptoms |
| | (orthopnea, easy | with progressive | (orthopnea and nocturnal |
| | fatigability) on maximal | symptoms (edema and | cough) on beta blocker and |
| | dose of beta blocker. | easy fatigability) on | ARNI. Known HF with |
| | Comorbid illnesses include | optimal therapy. On | reduced ejection fraction, |
| | HF with reduced ejection | history he has a recent | hypertension, and obesity. |
| | fraction, DM, chronic | urgent care visit for | Recently diagnosed with |
| | venous insufficiency, | osteoarthritic knee pain, | depression and prescribed |
| | dyslipidemia, overweight, | where he was prescribed | mirtazapine. Has also been |
| | depression, chronic kidney | celecoxib. He self- | taking NSAID sporadically |
| | disease stage 2. | medicated with an | for knee pain. |
| | | unrecalled medication for | |
| | | knee pain as well. | |
| | | Comorbid illnesses | |
| | | include hypertension, | |
| | | depression, and benign | |
| | | prostatic hypertrophy. | |
| Diabetes | 78-year-old woman on | 61-year old man with low | 67-year-old woman |
| (DM)/ | follow-up, with poorly- | back pain and dizziness. | complaining of headaches, |
| Hypertension | controlled hypertension on | Diagnosed with | with poorly controlled |
| (HTN) | telmisartan. On evaluation, | hypertension, diabetes, | hypertension on maximum |
| | patient also has mild | low back pain, | dose lisinopril/HCTZ. Has |
| | depression. | dyslipidemia. Recently | chronic Afib (on beta |
| | Comorbid illnesses include, | started an antibiotic for | blocker and rivaroxaban) |
| | chronic kidney disease | urinary tract infection. | with controlled heart rate. |
| | stage 3a, diabetes, anemia, | | Also diagnosed with |
| | dyslipidemia, osteoporosis, | | diabetes, obesity, and |
| | and osteoarthritis. | | chronic kidney disease |
| | | | stage 2. Currently taking an |
| | | | OTC supplement for |
| | | | wellness. |

### Study Outcomes and Analysis.

The study seeks to determine the clinical utility of the CDM test. Accordingly, the primary outcome is whether using CDM test improves patient care by increasing the diagnosis of NA or DDI or if it leads to changes in medication treatment. More specifically, we want to: (1) determine the change in frequency of identifying/diagnosing NA, DDIs, and AND; (2) measure the difference in treating these three patient types as a result of receiving the CDM test reports; and (3) explore how effective the intervention materials were in getting participants to order the CDM test. All primary outcomes are based on a 0%-100% scale, with primary analyses calculated as a difference-in-difference percentage between intervention and control and as an odds ratio with 95% confidence interval as determined by multiple variable logistic regression.

Secondary outcomes include the effect of provider and clinical practice characteristics on care, cost implications of using the CDM test, and identifying the best use cases of the CDM test. The effect of provider and clinical practice characteristics are determined by inserting these variables into the multiple variable regression models. Cost analysis is done by measuring differential rates of diagnostic ordering selected by each arm and multiplying by average Medicare reimbursement rates for these workups. Use case determination is made through difference-in-difference and logistic sub-analyses of the clinical variants presented to the participants.

All analyses are done in Stata 14.2.

### Study Results

### Physician Characteristics

The eligibility requirements are met by 236 board-certified PCPs (Table 3). The physician questionnaire and six CPV patient cases are completed by the 235 PCPs. Over half of the participants work in a suburban setting and a quarter work in solo private practice. Intervention arms have slightly more male participants (control: 68.4%; intervention 1: 77.9%; intervention 2: 75.9%; p=0.367) and intervention 1 have more internal medicine physicians (control: 54.0%; intervention 1: 61.0%; intervention 2: 51.8%; p=2.475).

TABLE 3
Physician Baseline Characteristics, by Study Arm.

| | Control | Intervention 1 | Intervention 2 | p-value |
|---|---|---|---|---|
| N | 76 | 77 | 83 | — |
| Male | 68.4% | 77.9% | 75.9% | 0.367 |
| Age | 56.1 ± 8.7 | 57.0 ± 7.8 | 56.3 ± 8.2 | 0.940 |
| Internal Medicine | 54.0% | 61.0% | 51.8% | 0.475 |
| Practice Type | | | | |
| Hospital-Based | 9.2% | 11.7% | 12.1% | 0.266 |
| Private, Multi-Specialty | 34.2% | 32.5% | 20.5% | |
| Private, Single Specialty | 30.3% | 29.9% | 45.8% | |
| Private, Solo | 26.3% | 26.0% | 21.7% | |
| Region* | | | | |
| Northeast | 33.3% | 24.7% | 28.9% | 0.829 |
| South | 28.0% | 32.5% | 33.7% | |
| Midwest | 21.3% | 20.8% | 22.9% | |
| West | 17.3% | 22.1% | 14.5% | |
| Setting* | | | | |
| Urban | 28.0% | 37.7% | 25.3% | 0.248 |
| Suburban | 61.3% | 54.6% | 57.8% | |
| Rural | 10.7% | 7.8% | 16.9% | |
| Employed by Practice* | 82.7% | 77.9% | 69.9% | 0.157 |
| Payer, % | | | | |
| Medicare | 37.7% | 34.5% | 36.5% | 0.654 |
| Medicaid | 10.2% | 8.7% | 9.1% | 0.571 |
| Commercial | 45.0% | 49.7% | 48.8% | 0.823 |
| Self | 4.5% | 5.0% | 6.4% | 0.252 |
| Other | 2.7% | 2.0% | 2.2% | 0.730 |
| Participant in CMS Quality Program* | | | | |
| Yes | 30.7% | 31.2% | 41.0% | 0.356 |
| No | 52.0% | 58.4% | 44.6% | |
| Don't know | 17.3% | 10.4% | 14.5% | |
| Medication Reconciliation Used | | | | |
| Pharmacy reconciliation | 90.8% | 89.6% | 88.0% | 0.843 |
| EMR alert | 85.5% | 80.5% | 75.9% | 0.310 |
| Self-report | 67.1% | 68.8% | 73.5% | 0.659 |
| Urine drug screen | 35.5% | 32.5% | 37.4% | 0.809 |
| Confirmation drug testing | 19.7% | 15.6% | 25.3% | 0.308 |
| Digital pills | 2.6% | 1.3% | 1.2% | 0.744 |
| None | 0.0% | 0.0% | 2.4% | 0.156 |

*One control participant missing data

### Diagnosis-and-Treatment Domain Scores

At baseline, we find wide variation in DxTx scores among participants caring for patients with cardiometabolic diseases. Across all patient cases, DxTx ranges from 0% to 75%, averaging 21.7%±13.4%. DxTx scores among the three case types are 22.7% for Afib, 22.4% for HF, and 19.9% for DM/HTN (p>0.05). Breaking out the results by the three case variants, we find participants diagnosed and treated NA 23.8%±13.4% more frequently than DDI 17.7%±14.1%, and about the same as AND 23.5%±11.8%; these differences are significant (p<0.001). There are no significant differences in DxTx scores between study arms at baseline (Table 4) (p>0.05 for all case variants).

TABLE 4
Diagnosis-Treatment Scores by CPV Variant.

| Study arm | R1 | R2 | p-value |
|---|---|---|---|
| Medication Nonadherence | | | |
| Control | 21.2% ± 12.1% | 24.0% ± 14.6% | 0.195 |
| Intervention 1 | 25.0% ± 13.2% | 38.2% ± 22.1% | <0.001 |
| Intervention 2 | 25.3% ± 14.4% | 30.4% ± 20.0% | 0.031 |
| p-value | 0.102 | <0.001 | 0.006/0.540 |
| Drug-drug Interaction | | | |
| Control | 16.1% ± 13.6% | 18.8% ± 13.7% | 0.216 |
| Intervention 1 | 17.4% ± 15.1% | 31.0% ± 18.4% | <0.001 |
| Intervention 2 | 19.4% ± 13.7% | 19.7% ± 17.9% | 0.449 |
| p-value | 0.331 | <0.001 | 0.003/0.487 |
| Disease Progression | | | |
| Control | 20.8% ± 10.9% | 23.6% ± 10.9% | 0.140 |
| Intervention 1 | 24.9% ± 12.0% | 20.1% ± 10.3% | 0.004 |
| Intervention 2 | 24.6% ± 11.4% | 21.5% ± 10.3% | 0.036 |
| p-value | 0.055 | 0.124 | 0.003/0.021 |

The control is compared to the first intervention in a pre-post analysis. The formal difference-in-difference estimations using a fixed effects model shows a +10.4% improvement in recognizing and treating NA and a +10.8% improvement for identifying and treating DDI (p<0.01 for both). There is no round over round improvement in the DxTx score for the AND patient cases.

After controlling for gender, internal medicine specialty, age, region, practice locale and type, the fixed-effects model shows that practicing in the West (+4.3%, 95% C.I.+2.2% to +6.4%) and in non-urban environments (+2.1%, 95% C.I.+0.3% to +3.9%) are correlated with higher DxTx scores. Comparing intervention to control, intervention 1 providers perform significantly better than controls across all patient cases (+4.5%, 95% C.I.+0.6% to +8.3%). By case variant, the intervention group improves significantly for both the NA (+10.8%, 95% C.I.+3.4% to +18.2%) and DDI cases (+11.0%, C.I.+4.2% to +17.9%), but not for the AND cases (−7.6%, 95% C.I.−12.6% to −2.6%).

By case type (Afib, HF, and DM/HTN), we find no significant improvement in DxTx score for the intervention group in the difference-in-difference fixed effects modeling (Afib: +2.9%; HF: +3.8%; DM/HTN: +6.7%; p>0.05 for all). However, we do see improved identification of both NA and DDI across all case types (Afib, O.R. 39.7, 95% C.I. 5.1-309.4; HF, O.R. 19.2, 95% C.I. 1.6-230.7; DM/HTN, O.R. 99.0, 95% C.I. 8.8-1179.5).

### Identification and Treatment of Underlying Cause by Case Variant

The combined DxTx score is disaggregated to explore how well physicians identified and managed the root problems of their patient's symptoms (NA, DDI, or disease progression).

Medication nonadherence—At baseline, among the NA patient cases, providers identify NA in their patients only 2.0% of the time, with no difference between study arms (p=0.414) (Table 5a). After introduction of the CDM test, intervention increases their detection of NA from 1.3% to 39.0% (p≤0.001), while control stayed nearly the same (2.6% to 2.7%, p=0.989).

The improvement in NA case diagnosis in intervention 1 leads to improved clinical care manifested by continuing medication and discussing the importance of medication adherence. After introduction of the CDM test, intervention continues the nonadherence medications by an additional 20.8% (p=0.003) (Table 5b). Control performance is unchanged at 6.6% and 5.3% round-to-round (p=0.747).

TABLE 5
Primary Variant Diagnosis and Related
Treatment, by Case Variant.

| | | |
|---|---|---|
| R1 | R2 | p-value |

(a) Primary Variant Diagnosis

| | | | |
|---|---|---|---|
| Nonadherence (Variant NA) | | | |
| Control | 2.6% | 2.7% | 0.989 |
| Intervention 1 | 1.3% | 39.0% | <0.001 |
| Intervention 2 | 4.8% | 14.8% | 0.031 |
| p-value | 0.414 | <0.001 | |
| DDI (Variant DDI) | | | |
| Control | 9.2% | 9.3% | 0.979 |
| Intervention 1 | 6.5% | 57.1% | <0.001 |
| Intervention 2 | 9.6% | 18.5% | 0.102 |
| p-value | 0.745 | <0.001 | |

Other (apixaban resistance, HF progression, HTN-caused symptoms)
(Variant AND)

| | | | |
|---|---|---|---|
| Control | 50.0% | 72.0% | 0.006 |
| Intervention 1 | 64.9% | 57.1% | 0.321 |
| Intervention 2 | 66.3% | 58.0% | 0.277 |
| p-value | 0.071 | 0.105 | |

(b) CMD-related Primary Treatment

| Continue stopped medication (Variant NA) | | | |
|---|---|---|---|
| Control | 6.6% | 5.3% | 0.747 |
| Intervention 1 | 14.3% | 35.1% | 0.003 |
| Intervention 2 | 9.6% | 13.6% | 0.430 |
| p-value | 0.283 | <0.001 | |

Stop/switch interacting substance (Variant DDI)

| | | | |
|---|---|---|---|
| Control | 32.9% | 12.0% | 0.002 |
| Intervention 1 | 32.5% | 64.9% | <0.001 |
| Intervention 2 | 39.8% | 21.0% | 0.009 |
| p-value | 0.553 | <0.001 | |

Other (shift med, advise HF progression, HTN workup) (Variant AND)

| | | | |
|---|---|---|---|
| Control | 14.5% | 32.0% | 0.011 |
| Intervention 1 | 23.4% | 28.6% | 0.462 |
| Intervention 2 | 25.3% | 33.3% | 0.258 |
| p-value | 0.211 | 0.803 | |

Regression modeling confirms that intervention is 50.4× more likely to identify NA (95% C.I. 2.9-871.2) and 3.3× more likely to continue the medications for which their patients are nonadherent, although the latter proves not to be significant (O.R. 0.6-19.3)

Drug-drug interactions—At baseline, identification of DDIs is modest in both control and intervention (7.8% for both arms, p=0.532). After introduction of the CDM test, intervention significantly improves their ability to identify DDIs, increasing from 6.5% to 57.1% (p<0.001) compared to control which did not change (9.2% to 9.3%, p=0.979).

After identifying more DDIs, intervention is nearly twice as likely to make a clinical adjustment by typically either stopping the interacting medications or shifting to a different medication (32.5% to 64.9%, p<0.001) compared to control (32.9% to 12.0%, p=0.002).

Here, the fixed effects model confirms that intervention is 26.9× more likely to identify the DDI (95% C.I. 5.6-130.6) and 15.7× more likely to stop the interacting substance (95% C.I. 5.0-49.0).

Disease progression—Although making the diagnosis of disease progression trends in the right direction for these patient cases at +3.2% this trend does not reach statistical significance (p=0.838) and, in the difference-in-difference estimation, the intervention group is 0.3× as likely to diagnose disease progression and 0.3× as likely to advance the medication regimen or increase medication dose.

### Intervention 2 Results

We want to determine whether the educational materials increases awareness of NA or DDIs and if so, how this impacts practice and test ordering by physicians.

Overall, intervention 2 only orders the CDM test in 12.4% of patient cases with no significant difference by case variant (p=0.892). When given the option of ordering the CDM test after reviewing the education materials, they have a nonsignificant improvement in their DxTx domain scores for NA cases of +2.0%, (p=0.542) and no improvement in the other case variants when compared to baseline scores. When we look at diagnosing NA, the second intervention improves from 4.8% to 14.8% (p=0.031), while the control remains the same (p=0.989). Intervention 2 also improves from 9.6% to 18.5% in diagnosing DDIs (p=0.102). When we control for physician and practice characteristics, we find a 3.6× (95% C.I. 0.3-34.8) improvement by intervention 2 to identify nonadherence in the NA patient cases and 1.9× (95% C.I. 0.4-9.8) improvement in treatment compared to controls. Similarly, for the DDI cases, intervention 2 is 2.3× (95% C.I. 0.5-9.4) more likely to identify the patient's DDI and 1.5× (95% C.I. 0.5-4.4) more likely to treat it.

Intervention 2 is split into two subgroups and those who choose to order the CDM test (Int2A) and those who did not order the test (Int2B) is analyzed. At baseline, there is no difference between the two subgroups. However, after introducing the education materials, Int2A is significantly better in DxTx (43.2%±24.8% vs. 21.2%±14.0%), making the appropriate primary diagnosis (60.0% vs. 18.9%, p<0.001), and in discontinuing the offending agent(s) (50.0% vs. 18.2%, p<0.001) compared to Int2B.

Int2A has similar scores to intervention 1, and Int2B scores similarly to control (Table 6). Int2A, like intervention 1, is significantly more likely to improve DxTx scores in the NA (+32.4%) and DDI (+20.7%) patient cases but not in the AND cases (−2.4%). Int2A is also significantly more likely to identify NA (O.R. 41.6×) and DDI (O.R. 56.2×) but not disease progression (O.R. 0.3×). In treatment, Int2A is significantly more likely to stop the interacting medicine (O.R. 14.0×) in the DDI patient cases, and although improvement is seen, Int2A is not significantly more likely to continue the stopped medication in the NA cases (O.R. 7.2×).

TABLE 6
Intervention 2: Comparison by CDM Usage Versus
Control, Multivariate Regression Modeling.

| | Intervention 2 | Intervention 2 |
|---|---|---|
| | w/CDM test | w/o CDM test |
| Change in DxTx Score, % | | |
| NA | +32.4%*** | −4.0% |
| DDI | +20.7%* | −5.0% |
| Disease progression | −2.4% | −6.4% |

Change in Primary Underlying Diagnosis, O.R.

| | | |
|---|---|---|
| NA | 41.6x* | 1.1x |
| DDI | 56.2x** | 1.1x |
| Disease progression | 0.3x | 0.3x |

Change in Primary Related Treatment, O.R.

| | | |
|---|---|---|
| NA | 7.2x | 0.7x |
| DDI | 14.0x* | 0.9x |
| Disease progression | 0.3x | 0.3x |

*p < 0.05;
**p < 0.01;
***p < 0.001

### Economic Changes in Diagnostic Ordering

The economic impact of the CDM test is examined, we find that intervention 1 physicians ordered 0.3 fewer low-value tests per case (95% C.I. 0.0 to 0.6). This decrease in test ordering translates to a per case savings of $119 (95% C.I. $20 to $217).

### Study Discussion

In patients with chronic cardiometabolic diseases, healthcare outcomes depend upon correct diagnoses and effective treatment regimens. NA and DDIs are underrecognized but significant barriers to effective medical treatment. Our earlier study among U.S. PCPs revealed that NA and DDIs was recognized and diagnosed in just 3.6% and 8.9% of cases, respectively despite 99% of participants indicating that they used some form of medication reconciliation in their everyday practice (Valdenor et al., “Clinical variation in the treatment practices for medication nonadherence, drug-drug interactions, and recognition of disease progression in patients with chronic cardiometabolic diseases: A cross-sectional patient simulation study among primary care physicians,” Int J Clin Pract 6450641(2022)). Treatment suffered, too: 24.4% of NA cases and 40.5% of DDI cases were inappropriately treated.

A RCT is conducted to determine if the CDM test improved the recognition, diagnosis, and medication management of NA and DDIs in patients with chronic cardiometabolic diseases. The results show large differences between the intervention and the control groups: physicians who used the CDM test are 50.4× more likely to diagnose NA and 26.9× more likely to diagnose DDIs compared to the control. Importantly, they also provide improved subsequent care: they are 3.3× more likely to restart the medication, the appropriate way to address NA, and 15.7× more likely to stop or switch the interacting medications, the appropriate treatment for DDIs. The difference-in-difference calculations for DxTx scores, our combined measure of diagnostic and therapeutic improvement, confirm this effect.

Although the CDM test can not explicitly test for disease progression, physicians can diagnose disease progression by deduction after the CDM test excludes NA and DDIs. Even though intervention physicians are more likely to identify disease progression, there is not a significant difference when compared to control in diagnosis or treating disease progression. Should future study confirm our findings that routine use of tools, such as the CDM test, can objectively exclude NA and DDI, a clinician's ability to determine whether worsening symptoms are due to worsening clinical conditions, ineffective medications, or misdiagnosis can be improved.

Interestingly, when we compare the overall scores between the three case types, Afib, HF, and DM/HTN, we find no overall differences in diagnosis and treatment between each of the case types in aggregate. We interpret this finding as an indicator of the overriding challenge physicians face when caring for patients with multiple chronic conditions and accompanying polypharmacy, regardless of the specific disease, indicating the CDM Test has value across all four disease states.

Overall, only 1 in 8 providers in the elective intervention group choose to order the CDM test, suggesting that even with education, a more compelling narrative on these challenges than presented in our education materials is needed to alert physicians. Notwithstanding, Int2A is significantly more likely to make the primary diagnosis (58.1% vs. 16.5%) and order the correct related treatment (48.4% vs. 22.1%), compared to Int2B. These results closely mirror the results we see in the first intervention, who are all given the result, and control who are not.

The potential economic impact of the CDM test is compelling. Intervention orders 0.3 fewer low-value diagnostic tests per case, leading to savings of $119. While savings through reduced utilization of low-value diagnostic testing is in line with the potential the CDM test costs, our analysis does not include larger direct costs; factoring in lower clinical, emergency, and hospital visits, the direct cost benefits would increase significantly. NA is estimated to cause 150,000 emergency room (ER) visits and over one million hospitalizations per year. DDIs, similarly, have been associated with 74,000 annual ER visits and 195,000 annual hospitalizations. According to a study, the average cost of an ER visit is $383; reducing ED visits from NA and DDIs by 20% would compel over $17.0 million in savings from improved identification and treatment of NA and DDI using the CDM Test. Hospital stays are estimated at $11,700/stay; if these hospitalizations are conservatively reduced by 10%, the CDM test would deliver a cost reduction of $1.2 billion for NA and $228 million for DDIs.

Our findings have important implications for patients. Similar to a previous study which only looked at DDIs in clinical practice, (Peabody et al., “Clinical utility of definitive drug-drug interaction testing in primary care,” J Clin Med. 7(11):384 (2018)) these data show physicians are not checking for NA and that the polypharmacy of chronic disease management is attended by unrecognized DDIs. When physicians are unable to distinguish between NA and/or DDIs versus disease progression, assigning the correct treatment for these conditions becomes overwhelmingly more difficult. After introducing an accurate, reliable, and standardized office-based test to identify NA and DDIs among patients with chronic cardiometabolic conditions, we see improvements not only in diagnostic accuracy but also, and perhaps more importantly, treatment for these common clinical conditions.

A careful effort is made to present cases of chronic cardiometabolic conditions commonly encountered in primary care, however, the nine cases used in this study could not cover all possible presentations of NA and DDIs. The CDM test incorporates a significant number of commonly encountered prescription and non-prescription substances capable of contributing to pharmacokinetic or pharmacodynamic interactions when taken with prescription medications used to treat cardiometabolic disease. This study uses CPV simulations which have been validated against actual practice in numerous studies.

### Example 2: Analytes Detected and Analyzed by the CDM Test

TABLE 7
Analytes used for detecting NA.

| | Parent | | |
|---|---|---|---|
| Analyte | Compound | Substance Class | Drug Class |
| Aclidinium | Aclidinium | Anticholinergic | Asthma/COPD |
| Albuterol/ | Albuterol/ | Beta Agonist | Asthma/COPD |
| Levalbuterol | Levalbuterol | | |
| Arformoterol/ | Arformoterol/ | Beta Agonist | Asthma/COPD |
| Formoterol | Formoterol | | |
| Beclomethasone | Beclomethasone | Corticosteroid | Asthma/COPD |
| Monopropionate | | | |
| Beclomethasone | Beclomethasone | Corticosteroid | Asthma/COPD |
| Budesonide | Budesonide | Corticosteroid | Asthma/COPD |
| Fluticasone Furoate | Fluticasone | Corticosteroid | Asthma/COPD |
| Fluticasone Propionate | Fluticasone | Corticosteroid | Asthma/COPD |
| Indacaterol | Indacaterol | Beta Agonist | Asthma/COPD |
| Methylprednisolone | Methylprednisolone | Corticosteroid | Asthma/COPD |
| Olodaterol | Olodaterol | Beta Agonist | Asthma/COPD |
| Roflumilast Metabolite | Roflumilast | Phosphodiesterase | Asthma/COPD |
| | | Inhibitor | |
| Roflumilast | Roflumilast | Phosphodiesterase | Asthma/COPD |
| | | Inhibitor | |
| Salmeterol | Salmeterol | Beta Agonist | Asthma/COPD |
| Vilanterol | Vilanterol | Beta Agonist | Asthma/COPD |
| Ipratropium | Ipratropium | Anticholinergic | Asthma/COPD |
| Tiotropium | Tiotropium | Anticholinergic | Asthma/COPD |
| Umeclidinium | Umeclidinium | Anticholinergic | Asthma/COPD |
| Acebutolol | Acebutolol | Beta Blocker | Cardiovascular |
| Amlodipine Metabolite | Amlodipine | Calcium Channel | Cardiovascular |
| | | Blocker | |
| Amlodipine | Amlodipine | Calcium Channel | Cardiovascular |
| | | Blocker | |
| Apixaban | Apixaban | Anticoagulants | Cardiovascular |
| Salicylic Acid | Aspirin | Antiplatelet | Cardiovascular |
| Atenolol | Atenolol | Beta Blocker | Cardiovascular |
| Atorvastatin Lactone | Atorvastatin | Statin | Cardiovascular |
| Atorvastatin | Atorvastatin | Statin | Cardiovascular |
| Azilsartan | Azilsartan | Angiotensin | Cardiovascular |
| | | Receptor Blocker | |
| Benazepril | Benazepril | ACE Inhibitor | Cardiovascular |
| Benazeprilat | Benazepril | ACE Inhibitor | Cardiovascular |
| Bendroflumethiazide | Bendroflumethiazide | Thiazide Diuretics | Cardiovascular |
| Betaxolol | Betaxolol | Beta Blocker | Cardiovascular |
| Bisoprolol | Bisoprolol | Beta Blocker | Cardiovascular |
| Bumetanide | Bumetanide | Loop Diuretic | Cardiovascular |
| Candesartan | Candesartan | Angiotensin | Cardiovascular |
| | | Receptor Blocker | |
| Captopril | Captopril | ACE Inhibitor | Cardiovascular |
| Carvedilol | Carvedilol | Beta Blocker | Cardiovascular |
| Chlorothiazide | Chlorothiazide | Thiazide Diuretics | Cardiovascular |
| Chlorthalidone | Chlorthalidone | Thiazide Diuretics | Cardiovascular |
| Clopidogrel COOH | Clopidogrel | Antiplatelet | Cardiovascular |
| Clopidogrel | Clopidogrel | Antiplatelet | Cardiovascular |
| Dabigatran | Dabigatran | Anticoagulants | Cardiovascular |
| Deacetyl Diltiazem-N- | Diltiazem | Calcium Channel | Cardiovascular |
| Oxide | | Blocker | |
| Diltiazem | Diltiazem | Calcium Channel | Cardiovascular |
| | | Blocker | |
| Dipyridamole | Dipyridamole | Antiplatelet | Cardiovascular |
| Enalapril | Enalapril | ACE Inhibitor | Cardiovascular |
| Enalaprilat | Enalapril | ACE Inhibitor | Cardiovascular |
| Eplerenone | Eplerenone | Aldosterone | Cardiovascular |
| | | Antagonist | |
| Eprosartan | Eprosartan | Angiotensin | Cardiovascular |
| | | Receptor Blocker | |
| Dehydro Felodipine | Felodipine | Calcium Channel | Cardiovascular |
| | | Blocker | |
| Felodipine | Felodipine | Calcium Channel | Cardiovascular |
| | | Blocker | |
| Fluvastatin | Fluvastatin | Statin | Cardiovascular |
| Fosinopril | Fosinopril | ACE Inhibitor | Cardiovascular |
| Fosinoprilat | Fosinopril | ACE Inhibitor | Cardiovascular |
| Furosemide | Furosemide | Loop Diuretic | Cardiovascular |
| Hydralazine Metabolite | Hydralazine | Vasodilator | Cardiovascular |
| Hydrochlorothiazide | Hydrochlorothiazide | Thiazide Diuretics | Cardiovascular |
| Indapamide | Indapamide | Thiazide Diuretics | Cardiovascular |
| Irbesartan | Irbesartan | Angiotensin | Cardiovascular |
| | | Receptor Blocker | |
| Isradipine | Isradipine | Calcium Channel | Cardiovascular |
| | | Blocker | |
| Labetalol | Labetalol | Beta Blocker | Cardiovascular |
| Lisinopril | Lisinopril | ACE Inhibitor | Cardiovascular |
| Losartan Metabolite | Losartan | Angiotensin | Cardiovascular |
| | | Receptor Blocker | |
| Losartan | Losartan | Angiotensin | Cardiovascular |
| | | Receptor Blocker | |
| Lovastatin acid | Lovastatin | Statin | Cardiovascular |
| Lovastatin | Lovastatin | Statin | Cardiovascular |
| Methyclothiazide | Methyclothiazide | Thiazide Diuretics | Cardiovascular |
| Metolazone | Metolazone | Thiazide Diuretics | Cardiovascular |
| alpha-Hydroxy- | Metoprolol | Beta Blocker | Cardiovascular |
| metoprolol | | | |
| Metoprolol | Metoprolol | Beta Blocker | Cardiovascular |
| Moexipril | Moexipril | ACE Inhibitor | Cardiovascular |
| Moexiprilat | Moexipril | ACE Inhibitor | Cardiovascular |
| Nadolol | Nadolol | Beta Blocker | Cardiovascular |
| Nebivolol | Nebivolol | Beta Blocker | Cardiovascular |
| Nicardipine | Nicardipine | Calcium Channel | Cardiovascular |
| | | Blocker | |
| Nifedipine Carboxylate | Nifedipine | Calcium Channel | Cardiovascular |
| | | Blocker | |
| Nifedipine | Nifedipine | Calcium Channel | Cardiovascular |
| | | Blocker | |
| Nisoldipine | Nisoldipine | Calcium Channel | Cardiovascular |
| | | Blocker | |
| Olmesartan | Olmesartan | Angiotensin | Cardiovascular |
| | | Receptor Blocker | |
| Perindopril | Perindopril | ACE Inhibitor | Cardiovascular |
| Pindolol | Pindolol | Beta Blocker | Cardiovascular |
| Pitavastatin | Pitavastatin | Statin | Cardiovascular |
| Pravastatin | Pravastatin | Statin | Cardiovascular |
| Propranolol | Propranolol | Beta Blocker | Cardiovascular |
| Quinapril | Quinapril | ACE Inhibitor | Cardiovascular |
| Quinaprilat | Quinapril | ACE Inhibitor | Cardiovascular |
| Ramipril Metabolite | Ramipril | ACE Inhibitor | Cardiovascular |
| Ramipril | Ramipril | ACE Inhibitor | Cardiovascular |
| Rivaroxaban | Rivaroxaban | Anticoagulants | Cardiovascular |
| Rosuvastatin | Rosuvastatin | Statin | Cardiovascular |
| Sacubitril Metabolite | Sacubitril | Neprilysin Inhibitor | Cardiovascular |
| Sacubitril | Sacubitril | Neprilysin Inhibitor | Cardiovascular |
| Simvastatin | Simvastatin | Statin | Cardiovascular |
| Simvastatin Acid | Simvastatin | Statin | Cardiovascular |
| Canrenoic Acid | Spironolactone | Aldosterone | Cardiovascular |
| | | Antagonist | |
| Canrenone | Spironolactone | Aldosterone | Cardiovascular |
| | | Antagonist | |
| Spironolactone | Spironolactone | Aldosterone | Cardiovascular |
| | | Antagonist | |
| Telmisartan | Telmisartan | Angiotensin | Cardiovascular |
| | | Receptor Blocker | |
| Ticagrelor | Ticagrelor | Antiplatelet | Cardiovascular |
| Timolol | Timolol | Beta Blocker | Cardiovascular |
| Torsemide | Torsemide | Loop Diuretic | Cardiovascular |
| Trandolapril | Trandolapril | ACE Inhibitor | Cardiovascular |
| Trandolaprilat | Trandolapril | ACE Inhibitor | Cardiovascular |
| Valsartan | Valsartan | Angiotensin | Cardiovascular |
| | | Receptor Blocker | |
| Norverapamil | Verapamil | Calcium Channel | Cardiovascular |
| | | Blocker | |
| Verapamil | Verapamil | Calcium Channel | Cardiovascular |
| | | Blocker | |
| 7-Hydroxywarfarin | Warfarin | Anticoagulants | Cardiovascular |
| Warfarin | Warfarin | Anticoagulants | Cardiovascular |
| Canagliflozin | Canagliflozin | SGLT2 Inhibitor | Diabetes |
| Dapagliflozin | Dapagliflozin | SGLT2 Inhibitor | Diabetes |
| Empagliflozin | Empagliflozin | SGLT2 Inhibitor | Diabetes |
| Glimepiride | Glimepiride | Sulfonylureas | Diabetes |
| Glipizide | Glipizide | Sulfonylureas | Diabetes |
| Glyburide Metabolite | Glyburide | Sulfonylureas | Diabetes |
| Glyburide | Glyburide | Sulfonylureas | Diabetes |
| Linagliptin | Linagliptin | DPP-4 Inhbitor | Diabetes |
| Metformin | Metformin | Biguanide | Diabetes |
| (Glucophage) | | | |
| Hydroxypioglitazone | Pioglitazone | Thiazolidinedione | Diabetes |
| Pioglitazone | Pioglitazone | Thiazolidinedione | Diabetes |
| Sitagliptin | Sitagliptin | DPP-4 Inhbitor | Diabetes |

TABLE 8
Analytes used for detecting DDI.

| Analyte | Parent Compound | Drug Class |
|---|---|---|
| Amiodarone (Cordarone) | Amiodarone | Antiarrhythmics |
| Desethylamiodarone | Amiodarone | Antiarrhythmics |
| Quinidine | Quinidine | Antiarrhythmics |
| Ranolazine (Ranexa) | Ranolazine | Antiarrhythmics |
| Citalopram | Citalopram/ | Antidepressants and |
| | Escitalopram | Antipsychotics |
| N-Desmethylcitalopram | Citalopram/ | Antidepressants and |
| | Escitalopram | Antipsychotics |
| Duloxetine (Cymbalta) | Duloxetine | Antidepressants and |
| | | Antipsychotics |
| Fluvoxamine (Luvox) | Flovoxamine | Antidepressants and |
| | | Antipsychotics |
| Fluoxetine (Prozac) | Fluoxetine | Antidepressants and |
| | | Antipsychotics |
| Norfluoxetine | Fluoxetine | Antidepressants and |
| | | Antipsychotics |
| Iloperidone (Fanapt) | Iloperidone | Antidepressants and |
| | | Antipsychotics |
| Lurasidone | Lurasidone | Antidepressants and |
| | | Antipsychotics |
| Nefazodone (Serzone) | Nafazone | Antidepressants and |
| | | Antipsychotics |
| Olanzapine | Olanzapine | Antidepressants and |
| | | Antipsychotics |
| Paroxetine (Paxil) | Paroxetine | Antidepressants and |
| | | Antipsychotics |
| 7-Hydroxy Quetiapine | Quetiapine | Antidepressants and |
| | | Antipsychotics |
| Norquetiapine | Quetiapine | Antidepressants and |
| | | Antipsychotics |
| Quetiapine | Quetiapine | Antidepressants and |
| | | Antipsychotics |
| 9-Hydroxyrisperidone | Risperidone | Antidepressants and |
| | | Antipsychotics |
| Norsertraline | Sertraline | Antidepressants and |
| | | Antipsychotics |
| Sertraline (Zoloft) | Sertraline | Antidepressants and |
| | | Antipsychotics |
| Trazodone | Trazodone | Antidepressants and |
| | | Antipsychotics |
| O-desmethylvenlafaxine | Venlafaxine/ | Antidepressants and |
| | Desvenlafaxine | Antipsychotics |
| Venlafaxine (Effexor) | Venlafaxine/ | Antidepressants and |
| | Desvenlafaxine | Antipsychotics |
| Vortioxetine | Vortioxetine | Antidepressants and |
| | | Antipsychotics |
| Aprepitant (Emend) | Aprepitant | Antiemetic and |
| | | Gastric Reflux |
| Cimetidine (Tagamet) | Cimetidine | Antiemetic and |
| | | Gastric Reflux |
| Fosamprenavir (Lexiva) | Fosamprenavir | Antiemetic and |
| | | Gastric Reflux |
| 4-Hydroxy | Omeprazole/ | Antiemetic and |
| Omeprazole Sulfide | Esomeprazole | Gastric Reflux |
| Omeprazole Sulfone | Omeprazole/ | Antiemetic and |
| | Esomeprazole | Gastric Reflux |
| Butalbital | Butalbital | Antiepileptics |
| Carbamazepine | Carbamazepine | Antiepileptics |
| Carbamazepine Epoxide | Carbamazepine | Antiepileptics |
| Phenobarbital | Phenobarbital | Antiepileptics |
| Phenytoin (Dilantin) | Phenytoin | Antiepileptics |
| Primidone (Mysoline) | Primidone | Antiepileptics |
| Atazanavir (Reyataz) | Atazanavir | Antimicrobials and |
| | | Antiretrovirals |
| Azithromycin | Azithromycin | Antimicrobials and |
| | | Antiretrovirals |
| N-desmethyl azithromycin | Azithromycin | Antimicrobials and |
| | | Antiretrovirals |
| Ciprofloxacin (Cipro) | Ciprofloxacin | Antimicrobials and |
| | | Antiretrovirals |
| Clarithromycin (Biaxin) | Clarithromycin | Antimicrobials and |
| | | Antiretrovirals |
| Cobicistat (Tybost) | Cobicistat | Antimicrobials and |
| | | Antiretrovirals |
| Darunavir (Prezista) | Darunavir | Antimicrobials and |
| | | Antiretrovirals |
| Delavirdine (Rescriptor) | Darunavir | Antimicrobials and |
| | | Antiretrovirals |
| 8-Hydroxyefavirenz | Efavirenz | Antimicrobials and |
| | | Antiretrovirals |
| Efavirenz (Sustiva) | Efavirenz | Antimicrobials and |
| | | Antiretrovirals |
| Erythromycin (Erythrocin) | Erythromycin | Antimicrobials and |
| | | Antiretrovirals |
| Etravirine (Intelence) | Etravirine | Antimicrobials and |
| | | Antiretrovirals |
| Fluconazole (Diflucan) | Fluconazole | Antimicrobials and |
| | | Antiretrovirals |
| Amprenavir | Fosamprenavir | Antimicrobials and |
| | | Antiretrovirals |
| Indanavir (Crixivan) | Indinavir | Antimicrobials and |
| | | Antiretrovirals |
| Itraconazole (Sporanox) | Itraconazole | Antimicrobials and |
| | | Antiretrovirals |
| 2-Hydroxyitraconazole | Itraconazole | Antimicrobials and |
| | | Antiretrovirals |
| Ketoconazole (Nizoral) | Ketoconazole | Antimicrobials and |
| | | Antiretrovirals |
| Levofloxacin/Ofloxacin | Levofloxacin/ | Antimicrobials and |
| | Ofloxacin | Antiretrovirals |
| Metronidazole | Metronidazole | Antimicrobials and |
| | | Antiretrovirals |
| 2-Hydroxynelfinavir | Nelfinavir | Antimicrobials and |
| | | Antiretrovirals |
| Nelfinavir (Viracept) | Nelfinavir | Antimicrobials and |
| | | Antiretrovirals |
| Posaconazole (Noxafil) | Posaconazole | Antimicrobials and |
| | | Antiretrovirals |
| Quinine | Quinine | Antimicrobials and |
| | | Antiretrovirals |
| Rifabutin (Mycobutin) | Rifabutin | Antimicrobials and |
| | | Antiretrovirals |
| Rifampin (Rifadin) | Rifampin | Antimicrobials and |
| | | Antiretrovirals |
| Rifapentine (Priftin) | Rifapentine | Antimicrobials and |
| | | Antiretrovirals |
| Hydroxy Ritonavir | Ritonavir | Antimicrobials and |
| | | Antiretrovirals |
| Ritonavir (Norvir) | Ritonavir | Antimicrobials and |
| | | Antiretrovirals |
| Saquinavir (Invirase) | Saquinavir | Antimicrobials and |
| | | Antiretrovirals |
| Sulfamethoxazole | Sulfamethoxazole | Antimicrobials and |
| | | Antiretrovirals |
| Tipranavir (Aptivus) | Tipranivir | Antimicrobials and |
| | | Antiretrovirals |
| Trimethoprim | Trimethoprim | Antimicrobials and |
| | | Antiretrovirals |
| Voriconazole (Vfend) | Voriconazole | Antimicrobials and |
| | | Antiretrovirals |
| Voriconazole N-Oxide | Voriconazole | Antimicrobials and |
| | | Antiretrovirals |
| Amlodipine (Norvasc) | Amlodipine | Cardiovascular |
| Amlodipine Metabolite | Amlodipine | Cardiovascular |
| Apixaban | Apixaban | Cardiovascular |
| Salicylic Acid | Aspirin | Cardiovascular |
| Chlorothiazide | Chlorothiazide | Cardiovascular |
| Clonidine | Clonidine | Cardiovascular |
| Clopidogrel | Clopidogrel | Cardiovascular |
| Clopidogrel COOH | Clopidogrel | Cardiovascular |
| Digoxin | Digoxin | Cardiovascular |
| Deacetyl Diltiazem-N-Oxide | Diltiazem | Cardiovascular |
| Diltiazem (Cardizem) | Diltiazem | Cardiovascular |
| Fenofibrate | Fenofibrate | Cardiovascular |
| Fenofibrate Metabolite | Fenofibrate | Cardiovascular |
| Gemfibrozil | Gemfibrozil | Cardiovascular |
| Hydrochlorothiazide | Hydrochlorothiazide | Cardiovascular |
| Lovastatin | Lovastatin | Cardiovascular |
| Lovastatin Acid | Lovastatin | Cardiovascular |
| Rivaroxaban | Rivaroxaban | Cardiovascular |
| Sacubitril | Sacubitril | Cardiovascular |
| Simvastatin | Simvastatin | Cardiovascular |
| Simvastatin Acid | Simvastatin | Cardiovascular |
| Ticagrelor | Ticagrelor | Cardiovascular |
| Triamterene | Triamterene | Cardiovascular |
| Norverapamil | Verapamil | Cardiovascular |
| Verapamil (Calan) | Verapamil | Cardiovascular |
| 7-Hydroxywarfarin | Warfarin | Cardiovascular |
| Warfarin | Warfarin | Cardiovascular |
| Enzalutamide (Xtandi) | Enzalutamide | Chemotherapeutic |
| | | Agents |
| Nilotinib (Tasigna) | Nilotinib | Chemotherapeutic |
| | | Agents |
| Pazopanib (Votrient) | Pazopanib | Chemotherapeutic |
| | | Agents |
| 3-Hydroxyguanfacine | Guanfacine | Cognitive |
| | | Enhancement |
| Guanfacine | Guanfacine | Cognitive |
| | | Enhancement |
| Hydroxypioglitazone | Pioglitazone | Diabetes |
| Pioglitazone (Actos) | Pioglitazone | Diabetes |
| Bergaptol | Grapefruit | Foods and |
| | Furanocoumarins | Supplements |
| Dihydroxybergamottin | Grapefruit | Foods and |
| | Furanocoumarins | Supplements |
| Dihydrokavain | Kava | Foods and |
| | | Supplements |
| Hyperforin | St. John's Wort | Foods and |
| | | Supplements |
| Avanafil (Stendra) | Avanafil | Miscellaneous |
| Colchicine | Colchicine | Miscellaneous |
| Cyclosporine | Cyclosporine | Miscellaneous |
| 7-Hydroxymethotrexate | Methotrexate | Miscellaneous |
| Methotrexate | Methotrexate | Miscellaneous |
| Tacrolimus | Tacrolimus | Miscellaneous |
| Celecoxib | Celecoxib | NSAID |
| Diclofenac | Diclofenac | NSAID |
| Ibuprofen | Ibuprofen | NSAID |
| Indomethacin | Indomethacin | NSAID |
| Ketorolac | Ketorolac | NSAID |
| Meloxicam | Meloxicam | NSAID |
| Naproxen | Naproxen | NSAID |
| Cyclobenzaprine | Cyclobenzaprine | Skeletal Muscle |
| | | Relaxant |
| Norcyclobenzaprine | Cyclobenzaprine | Skeletal Muscle |
| | | Relaxant |
| Dehydrotizanidine | Tizanidine | Skeletal Muscle |
| | | Relaxant |
| Tizanidine | Tizanidine | Skeletal Muscle |
| | | Relaxant |

### Example 3: A Study to Evaluate and Support the Clinical Utility of a Novel CDM Test in the Management of Patients with Cardiometabolic Disease and Polypharmacy

### Methods

### Study Overview

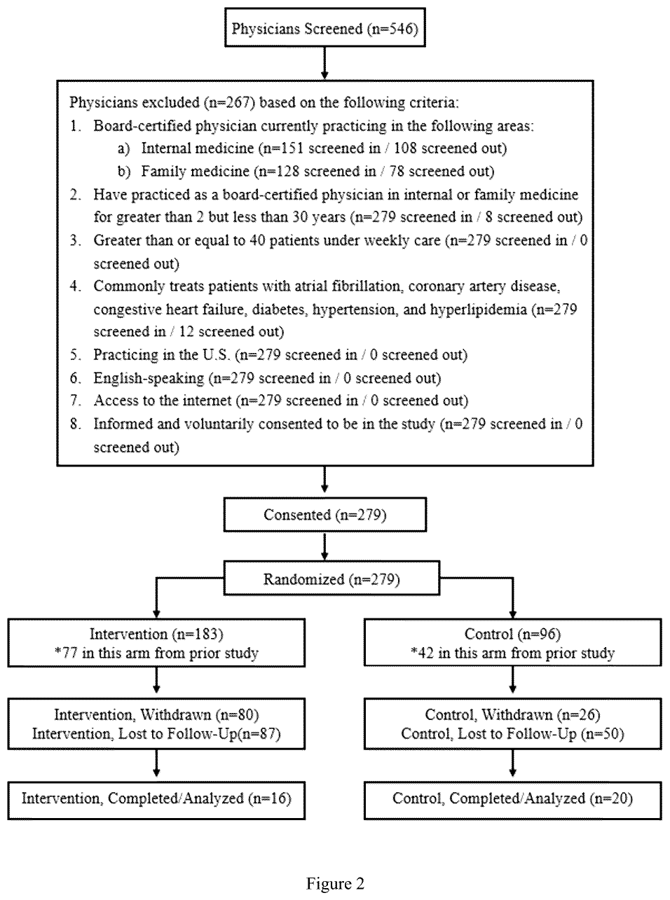

279 board-certified Primary Care Physicians (PCPs) are enrolled in a randomized controlled study to examine the clinical utility of the CDM test on PCPs' ability to detect and treat NA and DDI. PCPs randomized to the intervention arm (FIG. 2) are provided the opportunity to use the CDM test on patients in their practice who satisfied eligibility criteria (detailed below in “Patient Inclusion Criteria”) then, as appropriate, utilize the results to guide clinical decisions and actions. Both intervention and control PCPs provided patient data, at baseline and at least 3 months after the baseline visit, through de-identified medical record abstraction. PCPs randomized to the control arm use the same patient inclusion criteria but they are not provided with the CDM test. The primary outcomes include the ability of the intervention versus control PCPs to detect NA and DDI, as well as to demonstrate clinical utility of the CDM test as demonstrated by changes in patient care. The secondary outcome is the examination of the types of clinical actions performed.

### PCP Recruitment and Enrollment

PCPs are recruited from a previous virtual patient study, as well as additional PCPs who met the study inclusion criteria (FIG. 2) (Peabody et al., “Randomized prospective trial to detect and distinguish between medication nonadherence, drug-drug interactions, and disease progression in chronic cardiometabolic disease,” BMC Primary Care 24:100 (2023)). PCPs from the virtual patient study maintained their original randomization assignment (intervention or control) and newly recruited PCPs are randomized by a balanced randomized block size allocation (FIG. 2) (Efird J., “Blocked randomization with randomly selected block sizes,” Int J Environ Res Public Health 8(1):15-20 (2011)).

### Patient Inclusion Criteria

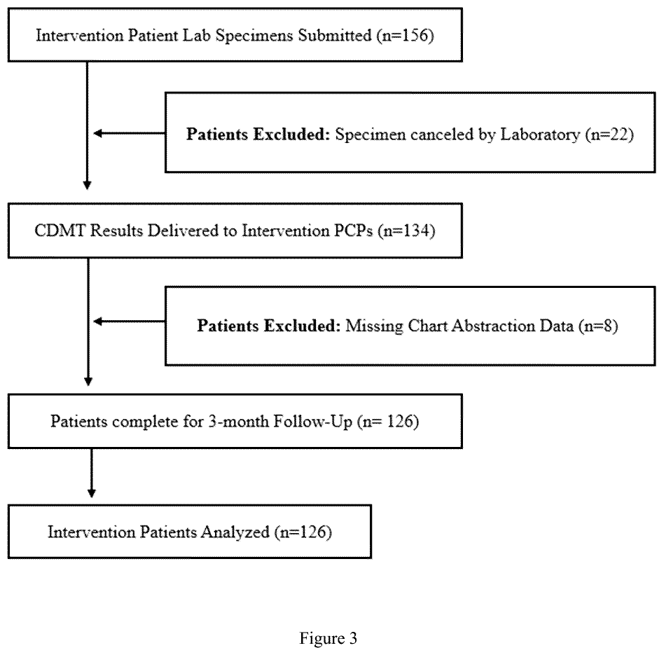

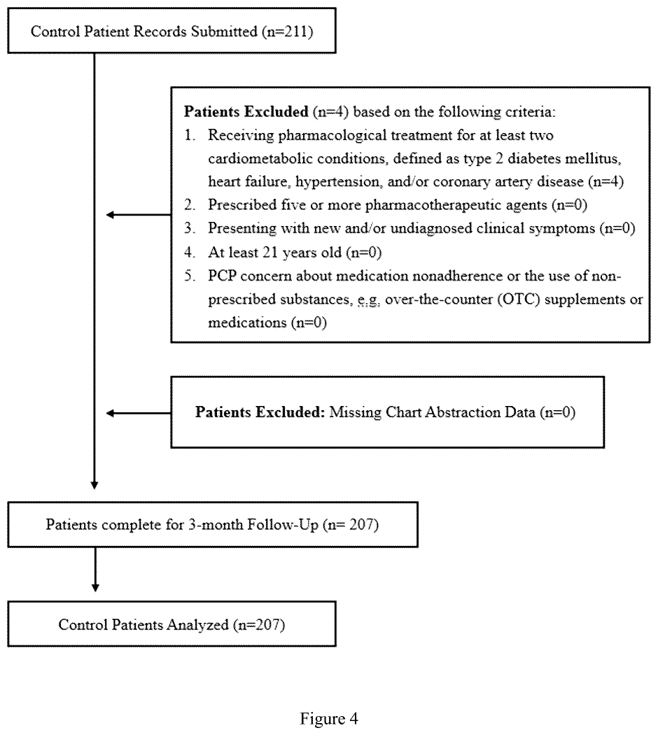

All PCPs are asked to identify 6-14 patients in their practice who satisfied the following inclusion criteria: (1) receiving pharmacological treatment for at least two cardiometabolic conditions: type 2 diabetes mellitus, heart failure, hypertension, and/or coronary artery disease; (2) being prescribed five or more drugs; (3) presenting with new and/or undiagnosed clinical symptoms; (4) being at least 21 years old, and (5) evoking PCP concern regarding NA, DDI, or the use of non-prescribed substances, e.g. over-the-counter supplements or medications. The patients who are determined to be eligible based on these criteria are detailed separately for the intervention PCPs (FIG. 3) and control PCPs (FIG. 4).

### Data Collection and Intervention

The primary data for this study is obtained from de-identified, abstracted patient medical records from both intervention and control PCPs. Medical record data are captured at baseline and at a follow-up occurring at least 3 months after the baseline data collection for each patient. The medical records are de-identified by the PCPs in compliance with section 164.514 of the U.S. Department of Health and Human Services' HIPAA Privacy Rule and were linked to CDM test results through a unique identification number. A third-party abstracter provided anonymized HIPAA-compliant medical records for review.

Patient consent from eligible patients is obtained by intervention PCPs for the CDM test according to their standard clinical practice. In-office saliva samples are obtained by the PCPs and the samples are submitted directly to the Aegis Sciences Corporation laboratory for complimentary analysis. CDM test results are returned to the PCPs.

### Primary and Secondary Outcomes

The primary outcomes are to determine if the CDM test results (1) improved PCPs' detection of NA and/or a DDI; and (2) led to the subsequent NA- or DDI-related clinical actions. The secondary outcome is to examine the ways in which intervention PCPs modified their clinical practice in the setting of CDM test results in order to address NA or DDI.

### Statistical Analyses

Single binary independent variables and categorical variables in intervention and control arms are analyzed using the chi-square test. Primary and secondary outcomes between study arms are analyzed by logistic regression using both a single variable and a clustered multivariate model, in order to adjust for potential patient characteristic similarities within PCP practice. The sample size calculation is based on a chi-square test of the ability of PCPs to detect NA and/or DDI among intervention patients compared to control patients. In order to achieve an alpha of 0.05 and a power of 80%, a baseline incidence of 20% and an effect size of 17.5% is assumed. Therefore, using standard power calculations, 104 patients is required to be enrolled in each study arm. However, in order to account for potential clustering effects within PCP practices, the sample size is increased by 20% to require a minimum sample size of 125 patients in each arm. All analyses are performed in Stata 18.0 (StataCorp. Stata Statistical Software: Release 18. College Station, TX: StataCorp, LLC (2023)).

### Results

### Physician Characteristics

After applying inclusion/exclusion criteria (FIG. 2), a total of 279 PCPs are enrolled and randomized. However, 106 PCPs are withdrawn from the study for reasons including, time constraints (n=34); institutional or organizational restrictions (n=27); personal circumstances or changes (n=12); lack of interest or willingness (n=18); misunderstanding of study expectations (n=7); miscellaneous or administrative issues (n=5); and technical or logistical issues (n=3). Additionally, 137 PCPs are lost to follow-up, resulting in 36 PCPs completing the study (16 intervention and 20 control). Comparing PCP characteristics between the two study arms, there are no significant differences by age, gender, years of experience, or practice demographics (P>0.05 for all; Table 9).

TABLE 9
Baseline characteristics of physicians. Data are percentages
within each category within each study population.

| | | |
|---|---|---|
| Intervention | Control | |
| (N = 16) | (N = 20) | P value |

Age Group (yrs)

| | | | |
|---|---|---|---|
| <40 | 6% | 10% | 0.85 |
| 40 to 55 | 56% | 60% | |
| >55 | 38% | 30% | |

Years of Experience

| | | | |
|---|---|---|---|
| 2 to 10 | 25% | 15% | 0.15 |
| 11 to 20 | 19% | 50% | |
| 21 to 35 | 56% | 35% | |

Sex

| | | | |
|---|---|---|---|
| Female | 6% | 25% | 0.19 |
| Male | 94% | 70% | |
| Prefer Not to Answer | 0% | 5% | |

Board Certification

| | | | |
|---|---|---|---|
| Family Medicine | 44% | 55% | 0.50 |
| Internal Medicine | 56% | 45% | |

Practice Region

| | | | |
|---|---|---|---|
| Northeast | 19% | 30% | 0.81 |
| South | 38% | 35% | |
| Midwest | 31% | 20% | |
| West | 12% | 15% | |

Practice Setting

| | | | |
|---|---|---|---|
| Urban | 25% | 45% | 0.38 |
| Suburban | 63% | 40% | |
| Rural | 12% | 15% | |

Practice Type

| | | | |
|---|---|---|---|
| Academic | 13% | 25% | 0.26 |
| Hospital-based | 25% | 0% | |
| Private | 62% | 70% | |
| Other | 0% | 5% | |

Patient Panel Size

| | | | |
|---|---|---|---|
| 500 to 1,000 | 13% | 5% | 0.33 |
| 1,001 to 2,000 | 31% | 55% | |
| >2,000 | 56% | 40% | |

### Patient Characteristics

After applying inclusion/exclusion criteria, a total of 126 intervention (FIG. 3) and 207 control patients (FIG. 4) are included in the analysis. When comparing patient characteristics between intervention and control study arms, respectively, no significant differences are found by age (mean±SD, 67.6±12.3 vs. 68.3±12.9 years); sex (male, 57.3 vs. 59.3%); or number of medications at baseline (11.5±6.9 vs. 10.7±6.2; all P>0.05; Table 10). When examining co-morbidities, no significant differences are found between the intervention and control patients by age, sex, number of medications taken at baseline, or prevalence of diseases. However, intervention patients are slightly more likely to have been diagnosed with non-cardiometabolic disease than control patients (71.6% vs. 63.8%; P=0.16).

TABLE 10
Baseline characteristics of patients.

| | Intervention (N = 126) | | Control (N = 207) | | P- |
|---|---|---|---|---|---|
| | Mean | SD | Mean | SD | Value |
| Age (yrs) | 67.6 | 12.3 | 68.3 | 12.9 | 0.66 |
| Male (%) | 57.3 | — | 59.3 | — | 0.73 |
| Baseline | 11.5 | 6.9 | 10.7 | 6.2 | 0.28 |
| Medications (n) | | | | | |

Comorbidity Frequencies (%)

| | | | | | |
|---|---|---|---|---|---|
| Hypertension | 87.3 | — | 85.5 | — | 0.65 |
| Diabetes | 65.1 | — | 58.9 | — | 0.26 |
| CAD | 21.4 | — | 21.8 | — | 0.95 |
| Heart Failure | 15.9 | — | 12.6 | — | 0.40 |
| Non-Cardiometabolic | 71.6 | — | 63.8 | — | 0.16 |
| Diseases | | | | | |

### CDM Test Results

Of the 126 intervention patients with CDM test results, 42.1% (53/126) have received results indicating NA alone, 9.5% (12/126) have received results indicating DDI alone, and 42.9% (54/126) have received results indicating the presence of both. Consequently, only 5.6% (7/126) of the CDM test results are negative for both NA and DDIL When examining NA, the CDM test has detected 3.1±1.8 (mean±SD) analyte abnormalities (i.e., findings that are not in alignment with the patient's treatment (e.g., NA to a prescribed medication, detection of a medication not indicated as prescribed)), with an average of 2.0±1.5 prescribed but not detected and 1.1±1.4 detected but not prescribed. Regarding DDI, the CDM test has detected 2.9±2.6 moderate or severe DDIs per test.

### NA and DDI Detection

At baseline, the percentages of intervention and control PCPs who identified NA or DDI in their patients are inferred from the PCP clinical actions performed (e.g., noted in chart, counseled patient, augmented or discontinued pharmacotherapy or other non-prescription substance use). At 3-month follow-up, after the CDM test results are shared, 107 patients are recognized within medical records as NA (P<0.001). Regarding DDI, after the CDM test results are shared, 66 patients are identified (P<0.001). Of note, nine NA-related actions are performed among the 19 intervention patients without CDM test-detected NA. Also, two DDI-related actions are performed among the 60 intervention patients without CDM test confirmation of DDI.

When considering the impact of the CDM test on clinical actions among the intervention PCPs, the clinical actions performed for NA are increased significantly, from 18.3% to 69.1% (P<0.001; Table 11a). Similarly, the clinical actions to address DDI are increased significantly, from 0.8% to 37.3% (P<0.001; Table 11b).

TABLE 11
Clinical actions taken in response to (a) NA or (b) DDI.

| | | | | Logistic Regression | | | |
|---|---|---|---|---|---|---|---|
| | | P- | Difference -in- | | 95% | 95% | P- |
| Intervention | Control | Value | Difference | O.R. | Lower | Upper | Value |

(a) NA

| | | | | | | | | |
|---|---|---|---|---|---|---|---|---|
| Baseline | 18.3% | 18.4% | 0.98 | | | | | |
| Follow-up | 69.1% | 20.3% | <0.001 | 48.9% | 8.8 | 4.1 | 19.0 | <0.001 |
| P-value | <0.001 | 0.62 | | | | | | |

(b) DDI

| | | | | | | | | |
|---|---|---|---|---|---|---|---|---|
| Baseline | 0.8% | 1.9% | 0.41 | | | | | |
| Follow-Up | 37.3% | 0.5% | <0.001 | 38.0% | 301.9 | 15.4 | 5904.7 | <0.001 |
| P-Value | <0.001 | 0.18 | | | | | | |

### NA-Related and DDI-Related Action Frequency

For NA, 18.3% of intervention patients compared to 18.4% of control patients (P=0.98) have a NA-related action performed at baseline, such as counseling, noting nonadherence on chart, adjusting/switching medications causing nonadherence, or offering tools to mitigate nonadherence (Table 11a). At 3-month follow-up, intervention patients receive a NA-related action 69.1% of the time compared to 20.3% of the time for control patients (P<0.001), representing a difference-in-difference of 48.9%. Consequently, the intervention arm is more likely to be associated with a change in clinical practice than the control arm (odds ratio, 8.8; 95% confidence interval [CI], 4.1 to 19.0; P<0.001). A full logistic regression model accounting for provider, patient, and practice characteristics, as well as potential PCP practice clustering effects, reveals an odds ratio of 11.9; 95% CI, 2.8 to 51.4; P<0.001).

Regarding DDI, only 0.8% of intervention patients have a DDI-related action performed at baseline compared to 1.9% of control patients (p=0.41; Table 11b). These DDI-relevant clinical management actions include counseling, noting new DDI in the patient's chart, adjusting/switching medications causing DDI, noting new medication allergy in chart, or offering a monitoring plan for potential increased symptoms of DDI. At 3-month follow-up, intervention patients receive a DDI-related action 37.3% of the time compared to 0.5% of the time for control patients (p<0.001), demonstrating a difference-in-difference of 38.0%. Simple logistic regression shows an odds ratio of 301.9 (95% CI, 15.4 to 5904.7). A full logistic regression model using the same variables as those used for the NA-related actions exhibits an odds ratio of 372.2 (95% CI, 16.6 to 8351.9).

### Types of NA-Related Actions

At baseline, there is no significant difference between intervention and control patients in the types of NA-related actions that are performed (P=0.20, Table 12). Nonetheless, the intervention compared to the control PCPs more frequently counseled their patients (91.3% vs. 73.3%), but less frequently adjusted their patients' medications (0.0% vs. 10.5%) or offered a tool to improve adherence, such as smart reminders, alarm setting, or digital pills (0.0% vs. 5.3%).

TABLE 12
Types of NA and DDI actions taken at baseline and follow-up. Providers
could perform more than one action for a given patient.

| Baseline | | | 3-Month Follow Up | | |
|---|---|---|---|---|---|
| Intervention | Control | P value | Intervention | Control | P value |

Medication Nonadherence

| | | | | | | |
|---|---|---|---|---|---|---|
| Number of Patient Actions | 23 | 38 | — | 87 | 42 | — |

Action Taken

| | | | | | | |
|---|---|---|---|---|---|---|
| Offered Counseling on | 91.3% | 73.7% | 0.20 | 77.0% | 90.5% | 0.04 |
| Medication Adherence | | | | | | |
| Adjusted the Medication | 0.0% | 10.5% | | 24.1% | 9.5% | |
| Regimen (changed dosage or | | | | | | |
| frequency, stopped, or | | | | | | |
| switched to an alternative | | | | | | |
| medication) | | | | | | |
| Noted Medication | 21.7% | 31.6% | | 31.0% | 14.3% | |
| Nonadherence on Chart | | | | | | |
| Offered Tool to Improve | 0.0% | 5.3% | | 0.0% | 0.0% | |
| Adherence | | | | | | |

DDI

| | | | | | | |
|---|---|---|---|---|---|---|
| Number of Patient Actions | 1 | 4 | — | 47 | 1 | — |

DDI Action

| | | | | | | |
|---|---|---|---|---|---|---|
| Offered Counseling on DDI | 0.0% | 0.0% | 0.12 | 63.8% | 0.0% | 0.003 |
| Adjusted the Medication | 0.0% | 0.0% | | 25.5% | 0.0% | |
| Regimen (changed dosage or | | | | | | |
| frequency, stopped, or | | | | | | |
| switched to an alternative | | | | | | |
| medication) | | | | | | |
| Noted DDI on Chart | 100.0% | 25.0% | | 34.0% | 0.0% | |
| Offered Monitoring Plan for | 0.0% | 0.0% | | 8.5% | 0.0% | |
| ADRs | | | | | | |
| Noted New Drug Allergy on | 0.0% | 100.0% | | 6.4% | 100.0% | |
| Chart (New Drug Allergy is | | | | | | |
| e.g. newly identified adverse | | | | | | |
| drug effect and/or therapeutic | | | | | | |
| non-response) | | | | | | |

After introduction of the CDM test, the intervention compared to the control PCPs have a different distribution of NA-related action (Table 12; P=0.04). Specifically, intervention PCPs are less likely to counsel their patients (77.0 vs. 90.5%) but are more likely to adjust their patients' medication regimen (24.1% vs. 9.5%) and note NA on the patient chart (31.0% vs. 14.3%). None of the PCPs at follow-up has offered an auxiliary tool (e.g., smart reminders, alarm settings, digital pills) that might improve medication adherence.

### Types of DDI-Related Actions

At baseline, there are no significant differences between intervention and control patients in the types of DDI-related actions performed (P=0.12, Table 12). At 3-month follow-up, the intervention PCPs offer counseling 63.8% of the time; adjust DDI-related medications 25.5% of the time; note a new DDI on the patient's chart 34.0% of the time; implement a monitoring plan 8.5% of the time; and note a new drug allergy 6.4% of the time (P=0.003, Table 12). Only one control patient receives a DDI-related action at 3-month follow-up, the notation of a new drug allergy.

### NA-Related and DDI-Related Actions in Non-Positive CDM Test Results

Among the 19 intervention patients without CDM test-detected NA, counseling is performed seven times, and a chart notation is performed two times. Among the 60 control patients without CDM test-detected DDI, a drug allergy is noted on the chart two times.

### Discussion

This randomized controlled study is conducted in order to quantify the ability of PCPs to detect NA and DDI, and to adjust clinical management accordingly. In addition, the types of responses PCPs offered in these two ADR scenarios is analyzed. These study aims are developed because, first, it is acknowledged that the highest rates of NA have been reported in adults taking medications for cardiovascular disease and diabetes; current approaches to detect NA are limited; and an expert consensus panel determined the need for research to develop a provider- and patient-friendly approach to accurately distinguish NA and DDI (Wong et al., “The association between multimorbidity and poor adherence with cardiovascular medications,” Int J Cardiol. December 15; 177(2):477-82 (2014); Vatcharavongvan et al., “Polypharmacy, medication adherence and medication management at home in elderly patients with multiple non-communicable diseases in Thai primary care,” Fam Med Prim Care Rev 19:412-6 (2017); Lam et al., “Medication Adherence Measures: An Overview,” Biomed Res Int. 2015:217047 (2015); Scheife et al., “Consensus recommendations for systematic evaluation of drug-drug interaction evidence for clinical decision support,” Drug Saf. February; 38(2):197-206 (2015)). Secondly, because the clinical utility of CDM test in scientifically validated virtual patients with chronic cardiometabolic disease has been demonstrated, the current study sets out to corroborate the virtual patient evidence among actual patients, with similar clinical characteristics (Peabody et al., “Randomized prospective trial to detect and distinguish between medication nonadherence, drug-drug interactions, and disease progression in chronic cardiometabolic disease,” BMC Primary Care 24:100 (2023)).

No significant difference is observed at baseline between the intervention and control PCPs in their ability to detect and adjust clinical management due to NA or DDI. Although the NA and DDI baseline data are not validated by CDM test, these data are inferred from the PCPs' clinical actions data, reflecting their customary approach to ADR management related to NA and DDIs. Additionally, these inferred data are believed to be valuable since the literature indicates that the highest rates of NA are detected by patient self-report (Foley et al., “Prevalence and predictors of medication non-adherence among people living with multimorbidity: a systematic review and meta-analysis,” BMJ Open September 2; 11(9):e044987 (2021)). Nonetheless, these self-reported rates of NA are still relatively low and support the need for enhanced provider and patient responsibility in fostering more effective discussions about medication adherence (Tarn et al., “Provider views about responsibility for medication adherence and content of physician-older patient discussions,” J Am Geriatr Soc. June; 60(6):1019-26 (2012)). Data from this study further confirm the presence of this clinical practice gap in that the control PCPs, who care for their patients without the use of CDM test, perform fewer clinical actions addressing these two ADRs than the intervention PCPs.

This study demonstrates that the CDM test is associated with (1) increasing the rate of NA and DDI detection, and (2) more clinically rigorous actions among intervention versus control PCPs. At baseline, the clinical actions are primarily counseling (91%) and chart documentation (22%); there are no medication adjustments (change dosage or frequency, stop, or switch to an alternative medication) made. At 3-month follow-up, after the CDM test, the clinical actions are distributed among counseling (77%), medication adjustment (24%), and chart documentation (31%). The shift towards more clinically impactful actions may reflect a greater degree of confidence regarding the certainty of NA status.

The use of the CDM test improves the intervention PCPs' ability to take clinical actions related to NA from 18.3% to 69.1% (P<0.001; Table 11a) and DDI from 0.8% to 37.3% (P<0.001; Table 11b). These data suggest that the detection of NA and DDIs is not being sufficiently recognized or treated in standard primary care. A few providers in this study exhibit a blanket patient education and counseling approach, regardless of patient presentation or results, however, the CDM test's influence on personalized healthcare decision-making related to polypharmacy remains significant.

Limited DDI detection is observed and clinical management actions performed at baseline among the intervention PCPs, providing further evidence regarding the diagnostic challenge posed by DDI assessment in clinical practice. While practicing PCPs may have access to electronic DDI databases, challenges in technical or time-dependent barriers likely still prevent more systematic use (Roblek et al., “Drug-drug interaction software in clinical practice: a systematic review,” Eur J Clin Pharmacol. February; 71(2):131-42 (2015); Kheshti et al., “A comparison of five common drug-drug interaction software programs regarding accuracy and comprehensiveness,” Journal of research in pharmacy practice, 5(4), 257 (2016); Sheikh-Taha et al., “Polypharmacy and severe potential drug-drug interactions among older adults with cardiovascular disease in the United States,” BMC Geriatr 21, 233 (2021)).

Notably, after the CDM test results are provided, intervention PCPs engage in the full range of actions, primarily counseling (64%); chart documentation (40%); medication adjustments (26%); and implementation of a DDI monitoring plan (8.5%; P=0.003, Table 12). These data suggest that the provision of an uncomplicated patient- and provider-friendly tool may support improved clinical management.

Interestingly, although a small number of control PCPs offer patients tools to improve medication adherence (e.g., smart reminders, alarm setting, digital pills) at baseline, no control PCPs elect to utilize these tools at 3-month follow-up. This preference may reflect a prior unsatisfactory experience with these devices or perhaps a lack of experience with them. While further investigation may be conducted to understand this observation, the unfortunate current reality is that there are limited resources utilized by PCPs to support NA detection.

### Conclusion

The optimal clinical management of patients with chronic cardiometabolic disease and polypharmacy requires awareness and active clinical management of NA and DDIs. Because of the limited and variable efficacy of patient self-reporting, monitoring systems, and currently available management tools, the use of a provider- and patient-friendly diagnostic tool is necessary. This study provides evidence for the clinical utility of a noninvasive test that can enable physicians to better detect NA and DDI, and to subsequently perform clinical actions that will promote improved clinical outcomes.

EXAMPLE 4: A study to evaluate a novel CDM test that uses an oral sample from a patient to detect NA and DDIs. The purpose of the study is to determine the clinical utility of the CDM test in the management of patients with chronic disease.

### Methods

### Study Overview

150 VillageMD primary care providers are recruited and asked to voluntarily participate in a randomized controlled trial (RCT) using simulated Clinical Performance and Value® (CPV®) vignettes. The study assesses the ability of providers to recognize NA, DDIs and disease progression, to determine the potential utility of the novel CDM test.

### Study Ethics

This study is conducted in accordance with ethical standards approved by the Advarra Institutional Review Board, Columbia, MD, USA, and accordingly listed on ClinicalTrials.gov (NCT05658653). Voluntary informed consent is obtained from all participants before study commencement.

### Participant Sample

All established VillageMD providers are invited to participate in the study. Of these, the first 150 practicing primary care providers (physicians and advanced practice providers [nurse practitioners and physician assistants]) who met eligibility criteria are serially enrolled. The eligibility criteria including:
licensed primary care provider (MD, DO, NP, or PA) currently practicing in General Internal Medicine or Family Medicine;
in practice as a licensed primary care provider for greater than two but less than 30 years;
currently caring for 40 or more patients weekly;
commonly treating patients with primary diagnoses of congestive heart failure and/or COPD and common comorbidities, including diabetes, hypertension, and atrial fibrillation;
practicing in the US;
English-speaking;
have access to broadband internet; and
voluntarily consented to be a study participant.

Primary care provider potential participants are identified via an inclusive roster provided by VillageMD leadership. Potential participants are contacted by email using a recruitment letter.

### Data Sources

This study includes two primary sources of data: a participant questionnaire and the providers' responses to CPVs. The questionnaire probes demographics, details of medical practice, location of practice, practice type, and patient make-up, among other items. The CPVs evaluate clinical practice.

### Clinical Performance and Value (CPV®) Vignettes

CPVs are a validated methodology, widely used to rapidly measure physician care decisions. CPVs use open-ended questions simulating typical patient encounters, with questions divided into five domains of care: (1) medical history taking, (2) physical examination, (3) diagnostic work-up, (4) making a diagnosis, and (5) management plan and patient monitoring.

### CPV Patient Cases

Nine simulated CPV vignettes are created in a matrix with 3 cases: (1) heart failure with reduced ejection fraction (HFrEF), (2) heart failure with preserved ejection fraction (HfpEF), and (3) COPD (See Table 13 for case matrix and patient details). All patients have comorbid conditions such as diabetes mellitus, chronic kidney disease, depression, etc., and are symptomatic at their outpatient visits. Each case has three variants (A) NA no DDI; (B) DDI no NA; (C) No NA nor DDI; disease progression likely. To assess their current practice when treating HF and COPD, providers complete, in random order, three CPV cases—one from each case type and case variant. This is done at baseline and in a second round of data collection to determine if participants could ascertain whether patients are experiencing NA and/or a DDI, or disease progression.

TABLE 13
CPV simulated patient case development matrix.

| | | CASE VARIANTS | | |
|---|---|---|---|---|
| | | | | Variant C |
| | | | Variant B | Not meeting treatment |
| | | Variant A | Not meeting treatment | goal; NA, DDI suspected |
| | | Test confirms medication | goal Patient at risk | Test indicates adherence |
| | | non-adherence (NA) No | for ADE or therapeutic | to medication as |
| | | drug-drug interaction (DDI) | failure due to DDI | prescribed and no DDI |
| Case Types | Case 1 | Case 1A | Case 1B | Case 1C |
| | Heart failure with reduced | See simulated patient | See simulated patient | See simulated patient |
| | ejection fraction (HFrEF) | details below | details below | details below |
| | Case 2 | Case 2A | Case 2B | Case 2C |
| | HFpEF (Heart failure with | See simulated patient | See simulated patient | See simulated patient |
| | preserved ejection fraction) | details below | details below | details below |
| | Case 3 COPD | Case 3A | Case 3B | Case 3C |
| | | See simulated patient | See simulated patient | See simulated patient |
| | | details below | details below | details below |

| | |
|---|---|
| Expected Physician Behaviors WITH CDM Test | Expected Physician Behaviors WITHOUT CDM Test |

Case 1A
Demographics: 67/F with progressive failure symptoms (orthopnea, easy fatigability) on maximal GDMT dose of bisoprolol
Comorbid illnesses: HFrEF, DM, chronic venous insufficiency, dyslipidemia, overweight, depression, CKD
Medications: bisoprolol, sacubitril/valsartan, metformin/empagliflozin, atorvastatin, escitalopram, furosemide PRN
Risk factors: age, polypharmacy, multiple co-morbidity, elevated LDL, overweight, smoker

| | |
|---|---|
| More likely to recognize NA | More testing (e.g., pro-BNP, 2D echo) |
| Advise about importance of adherence, aids/tools to | Increased subspecialty referral (Cardiology) |
| increase adherence | Possible invasive interventions (angiography, etc.) |
| Advise about DDI risk | |

Case 1B
Demographics: 69/M with known HF (on metoprolol succinate, valsartan) who presents with persistent failure symptoms
(edema, shortness of breath, and easy fatigability). On history he has a recent urgent care visit for knee
pain (has osteoarthritis) - was prescribed celecoxib. He self-medicated with ibuprofen (this is not known to
physician, will be revealed by CDM test) for knee pain also (has used his son's medications in previous occasions)
Comorbid illnesses: heart failure with reduced ejection fraction (HFrEF), hypertension, osteoarthritis, depression, BPH
Medications: metoprolol succinate, valsartan, rosuvastatin, furosemide PRN for edema, tamsulosin
Risk factors: age, polypharmacy, overweight, ethnic minority, multiple co-morbidity

| | |
|---|---|
| More likely to recognize DDI | More testing (2D echo to recheck EF; CAD testing) |
| Discontinue interacting medications | Medication adjustment (increase furosemide, start |
| Maximize non-pharmacologic treatment for OA | SGLT2 inhibitor, additional anti-HTN agent) which |
| Advise about DDI risk | may lead to increased adverse effects |
| | Increased subspecialty referral (Cardiology) |

Case 1C
Demographics: 64/F on follow-up after ED visit. Had progressive shortness of breath, exertional dyspnea, and cough
with occasional sputum production (whitish, no change in character) over the past month; no fever, no bipedal
edema. Seen at ED 1 week ago, managed as HF decompensation: beta blockers put on hold and prescribed short-course
diuretics. Currently still with cough and shortness of breath. PE: tachycardic, wheezing on auscultation
but no rales; no neck vein engorgement, no bipedal edema.
Comorbid illnesses: HFrEF from IHD diagnosed 4 years ago, COPD Group B diagnosed 4 years ago; hypertension,
newly diagnosed depression, bilateral knee osteoarthritis
Medications:
Cardiac meds: bisoprolol (on hold), sacubitril/valsartan (optimal dose) atorvastatin, aspirin
For COPD: Fluticasone propionate/salmeterol 250/50 mcg 1 puff BID, albuterol PRN
Others: Recently started taking kava supplements to help with sleep; Takes NSAIDs sporadically for knee pain
Risk factors: ethnic minority, polypharmacy

| | |
|---|---|
| More likely to recognize disease progression | Possible invasive interventions (for knee pain) |
| More appropriate testing (2D echo will | Possible medication adjustment due to suspected DDI, |
| actually reveal improvement in EF; will | possibly even reducing dosages of HF meds |
| eventually be diagnosed with uncontrolled | Discontinue kava supplement (will not address dyspnea) |
| COPD) and intervention (revascularization) | |
| Uptitration of GDMT and COPD meds | |

Case 2A
Demographics: 78/F on follow-up, with poorly controlled hypertension on telmisartan, lives at home alone.
On evaluation, patient has mild depression (likely cause of non-adherence)
Comorbid illnesses: HTN, CKD, diabetes, HFpEF, GERD, depression
Medications: telmisartan, carvedilol, pravastatin, sitagliptin/metformin, omeprazole
Risk factors: older age, polypharmacy, multi-morbidity, poor social support, elevated LDL, ethnic minority

| | |
|---|---|
| More likely to recognize NA | More testing (for possible secondary hypertension) |
| Treat depression aggressively (potential | Additional BP medications such as diuretics |
| cause of non-adherence) | (which may lead to increased adverse effects |
| Advise about importance of adherence, aids/ | for patient) |
| tools to increase adherence, consider assisted | |
| living facility | |
| Advise about DDI risk | |

Case 2B
Demographics: 61/M, African American ancestry, presenting with fatigue
Comorbid illnesses: HFpEF, hypertension, diabetes, low back pain, dyslipidemia,
smokes 1 pack of cigarettes/day, carbuncle
Medications: losartan, metoprolol, metformin, glipizide, ibuprofen PRN, simvastatin,
trimethoprim/sulfamethoxazole
Risk factors: age, ethnic minority, multiple co-morbidity, recent ED visit for skin and
soft tissue infection, reduced coordination of care due to use of multiple providers

| | |
|---|---|
| More likely to recognize DDI | Hold losartan and/or metoprolol |
| Encourage non-pharmacologic interventions | Work up for HF progression, CAD |
| for smoking cessation | |
| Discontinue interacting medications | |
| Add a new antibiotic | |
| Add a different smoking cessation aid (varenicline) | |
| Advise about DDI risk | |

Case 2C
Demographics: 67/F complaining of headaches, with poorly controlled hypertension
Comorbid illnesses: HTN, DM, obesity, HFpEF
Medications: lisinopril (optimal dose), bisoprolol (optimal dose), metformin, linagliptin,
rosuvastatin, supplement for wellness containing elderberry
Risk factors: obesity, lower socioeconomic status, intake of herbal supplements, polypharmacy

| | |
|---|---|
| More likely to recognize disease progression | Increased work-up for headache (brain imaging) |
| Add another anti-hypertensive agent | Increased subspecialty referrals (neurology, |
| Work-up for secondary hypertension as cause | ophthalmology) |
| of HTN headache (e.g., renovascular disease) | Shifting to another medication or maintaining |
| Advise about progressive course of disease | current medication dose (when suspecting non- |
| Monitor blood sugar (look out for hypoglycemia) | adherence or DDI) - may not be enough to address |
| | hypertension |
| | Discontinue herbal supp. (will not address HTN) |

Case 3A
Demographics: 68/M on follow-up for COPD Group B. Reports recent increase in cough and sputum production.
Elevated BP 158/90.
Comorbid illnesses: hypertension, low back pain, GERD, depression (contributes to non-adherence), dyslipidemia
Medications: tiotropium 2.5 ug 2 puffs once daily, albuterol 90 mcg PRN, losartan 50 mg OD, simvastatin,
omeprazole, diclofenac gel
Risk factors: polypharmacy, multiple co-morbidity, lower socioeconomic status, higher co-pay

| | |
|---|---|
| More likely to recognize NA | Add LABA |
| Advise about importance of adherence, aids/ | Increase losartan dose |
| tools to increase adherence | Increased testing (infectious work-up, chest |
| Advise about DDI risk | imaging, pulmonary function tests) |

Case 3B
Demographics: 71/F with 5-day history of shortness of breath. Describes increased coughing and
shortness of breath 5 days ago, initially relieved by albuterol inhaler, but reports persistent
feeling of tiredness, like she just ran 4 blocks nonstop. PE and workup showed sinus
tachycardia, BP elevated from baseline at 146/90, others unremarkable.
Comorbid illnesses: COPD Group B, DM, dyslipidemia), hypertension, low back pain (on cyclobenzaprine)
Medications: albuterol 90 mcg, tiotropium 2.5 ug 2 puffs OD, canagliflozin/metformin, linagliptin,
lisinopril, carvedilol, atorvastatin, cyclobenzaprine, multivitamin
Risk factors: age, polypharmacy, multiple co-morbidity, ethnic minority

| | |
|---|---|
| More likely to recognize DDI | Increase medication dose |
| Discontinue interacting medication | Increased testing (workup for tachycardia) |
| Start new anti-HTN medication (CCB, diuretic) | |
| Optimize non-pharmacologic interventions for | |
| insomnia | |
| Optimize non-pharmacologic interventions for | |
| LBP | |
| Advise about DDI risk | |

Case 3C
Demographics: 66/M on follow-up for COPD Group B. Reports persistent symptoms on current therapy.
Had just shifted from umeclidinium to tiotropium 1 month ago due to difficulty using umeclidinium.
Also started on sumatriptan 5 days ago because of moderate-severe acute migraine attacks unrelieved
by NSAIDs/acetaminophen.
Comorbid illnesses: Hypertension (controlled), BPH, dyslipidemia, migraine headache
Medications: albuterol 90 mcg PRN, tiotropium inhalation spray 2.5 mcg 1 puff OD, valsartan, amlodipine,
tamsulosin, sumatriptan, atorvastatin, multivitamins
Risk factors: polypharmacy, multiple co-morbidity

| | |
|---|---|
| More likely to recognize disease progression | Discontinue sumatriptan or shift to another agent |
| Increase tiotropium and/or add LABA | |
| Continue sumatriptan | |
| Advise about progressive course of disease | |

Responses of each participant are independently scored by two trained physician experts using predetermined criteria based on current standards of care (see Table 14 and 15). In the case of disagreement in physician scoring, a third physician would serve as an adjudicator. A quality-of-care score (ranging from 0%, where the participant performs none of the standard-of-care criteria, to 100%, where the participant performs all of the standard-of-care criteria) is generated in each specific clinical domain of care: (1) medical history, (2) physical examination, (3) diagnostic work-up, (4) diagnosis, and (5) management plan and patient monitoring. Upon the scoring of these five domains, a combined overall quality-of-care score is calculated.

TABLE 14
Evidence-based items on diagnostics and management.

| Current standard of care | |
|---|---|
| (without CDM Test) | Reference(s) |
| Is it necessary to check | (Heidenreich et al. “AHA/ACC/HFSA |
| for medication adherence | Guideline for the Management of Heart |
| in patients with | Failure: A Report of the American |
| cardiometabolic conditions | College of Cardiology/American Heart |
| | Association Joint Committee on Clinical |
| | Practice Guidelines,” Circulation |
| | 145(18): e895-e1032 (2022); Venkatesan P, |
| | “GOLD COPD report: 2023 update,” |
| | Lancet Respir Med. 11(1): 18 (2023)) |
| Is it necessary to | (Fanciullo et al., “Best practices to |
| discontinue interacting | reduce the risk of drug-drug interactions: |
| medications | opportunities for managed care,” Am J |
| | Manag Care 17 Suppl 11: S299-304 (2011); |
| | Hansten et al., “ORCA: OpeRational |
| | ClassificAtion of drug interactions,” |
| | J Am Pharm Assoc (Wash) 41(2): 161-5 |
| | (2001); Scheife et al., “Consensus |
| | Recommendations for Systematic |
| | Evaluation of Drug-Drug Interaction |
| | Evidence for Clinical Decision Support,” |
| | Drug Safety 38(2): 197-206 (2015)) |
| Is it necessary to perform | (Heidenreich et al., “AHA/ACC/HFSA |
| therapeutic and non- | Guideline for the Management of Heart |
| pharmacologic management | Failure: A Report of the American College |
| for progressive heart failure | of Cardiology/American Heart Association |
| | Joint Committee on Clinical Practice |
| | Guidelines,” Circulation 145(18): |
| | e895-e1032 (2022)) |
| Is it necessary to perform | (Venkatesan P, “GOLD COPD report: 2023 |
| therapeutic and non- | update,” Lancet Respir Med. 11(1): 18 |
| pharmacologic management | (2023)) |
| for progressive COPD | |

TABLE 15
Specific scoring criteria for Case Type 1.

| | Variant A | | Variant B | | Variant C | |
|---|---|---|---|---|---|---|
| | Control | Intervention | Control | Intervention | Control | Intervention |
| | DIAGNOSTICS | | | | | |
| Necessary | 12L ECG | 12L ECG | Chest x-ray | Chest x-ray | 2D- | 2D- |
| | 2D- | 2D- | Serum chemistry | Serum chemistry | Echocardiography | Echocardiography |
| | Echocardiography | Echocardiography | | | (transthoracic | (transthoracic |
| | (transthoracic | (transthoracic | | | echocardiogram) | echocardiogram) |
| | echocardiogram) | echocardiogram) | | | A1c | A1c |
| | A1c | A1c | | | Chest x-ray | Chest x-ray |
| | BNP or NT- | BNP or NT- | | | Complete blood | Complete blood |
| | proBNP | proBNP | | | count with | count with |
| | Chest x-ray | Chest x-ray | | | Serum chemistry | Serum chemistry |
| | Complete blood | Complete blood | | | (including serum | (including serum |
| | count | count | | | phosphate) | phosphate) |
| | Serum chemistry | Serum chemistry | | | Thyroid function | Thyroid function |
| | Thyroid function | Thyroid function | | | tests | tests |
| | tests | tests | | | | |
| Appropriate | Lipid profile | Lipid profile | 12L ECG | 12L ECG | 12L ECG | 12L ECG |
| (not | Treadmill stress | Treadmill stress | 2D- | 2D- | BNP or NT- | BNP or NT- |
| required) | test | test | Echocardiography | Echocardiography | proBNP | proBNP |
| | Urinalysis | Urinalysis | (transthoracic | (transthoracic | Left heart | Left heart |
| | Urine | Urine | echocardiogram) | echocardiogram) | catheterization | catheterization |
| | albumin:creatinine | albumin:creatinine | A1c | A1c | Lipid profile | Lipid profile |
| | ratio | ratio | BNP or NT- | BNP or NT- | Spirometry | Spirometry |
| | (UACR) | (UACR) | proBNP | proBNP | Urinalysis | Urinalysis |
| | | | Complete blood | Complete blood | Urine drug | Urine drug |
| | | | count | count | testing | testing |
| | | | Lipid profile | Lipid profile | | |
| | | | Thyroid function | Thyroid function | | |
| | | | tests | tests | | |
| | | | Urinalysis | Urinalysis | | |
| Unnecessary | N/A | N/A | N/A | N/A | N/A | N/A |
| | MANAGEMENT | | | | | |
| Necessary | Advise about | Advise about | Increase | Advise about | Resume | Continue |
| | progressive | non-adherence | furosemide | DDI as cause | bisoprolol | fluticasone/ |
| | course of HF: | as cause of | (from as- | of symptoms, | Continue | salmeterol or |
| | Order CAD | symptoms of | needed to | uncontrolled | fluticasone/ | shift to |
| | testing | HF | standing doses) | BP | salmeterol | LAMA/LABA |
| | Cardiology | decompensation: | Add another | Discontinue | Continue | combination |
| | referral | Educate about | anti- | celecoxib | albuterol | Continue |
| | Short-course | importance of | hypertensive | Discontinue | Discontinue | albuterol |
| | furosemide | adherence, | agent: calcium | ibuprofen | kava kava | Start |
| | Add | aids/tools to | channel | Start | Cardiac | aldosterone |
| | mineralocorticoid | increase | blocker, | acetaminophen, | rehabilitation | antagonist |
| | receptor | adherence | thiazide | duloxetine, or | Pulmonary | Continue |
| | antagonist | Advise | Continue all | tramadol for | rehabilitation | sacubitril/ |
| | Add ivabradine | husband to help | other | chronic knee | Instruct patient | valsartan |
| | Increase | the patient and | maintenance | pain | to discontinue | Advise about |
| | furosemide | observe | medications | Add a topical | all NSAIDs | COPD |
| | (from as- | medication | (metoprolol, | NSAID | Referral to | exacerbation |
| | needed to | intake | valsartan, | Continue all | orthopedics for | Advise about |
| | standing doses) | Continue | rosuvastatin, | other | osteoarthritis | progressive |
| | Increase | sacubitril/ | tamsulosin) | maintenance | management | course of heart |
| | sacubitril/ | valsartan dose | Continue | medications | Maximize non- | failure (if with |
| | valsartan dose | Continue | celecoxib | (metoprolol, | pharmacologic | no |
| | Discontinue or | bisoprolol at | Maximize non- | valsartan, | treatment for | revascularization) |
| | lower dose of | current dose | pharmacologic | rosuvastatin, | osteoporosis | Discuss options |
| | bisoprolol | Continue all | treatment for | tamsulosin) | Follow up at 2 | for |
| | Continue all | other | osteoarthritis | Maximize non- | weeks or | revascularization |
| | other | maintenance | Order CAD | pharmacologic | longer | if |
| | maintenance | medications | testing | treatment for | | amenable or |
| | medications | (metformin/ | Cardiology | osteoarthritis | | Cardiology |
| | (metformin/ | empagliflozin, | referral | BP monitoring | | referral |
| | empagliflozin, | atorvastatin, | BP monitoring | (current BP is | | Cardiac |
| | atorvastatin, | escitalopram) | (current BP is | higher than | | rehabilitation |
| | escitalopram) | BP monitoring | higher than | usual) at home | | Pulmonary |
| | BP monitoring | Smoking | usual) at home | Follow-up | | rehabilitation |
| | Smoking | cessation, | Return visits 1- | phone call. | | Low salt and |
| | cessation, | weight loss, | 2 weeks for BP | Return in- | | protein |
| | weight loss, | salt restriction | check, | person visits in | | diet/nutrition |
| | salt restriction | Follow-up by | potassium | 1-3 months | | consult |
| | Return visits | phone in 2-4 | | | | Counseling to |
| | within 1 week | weeks. | | | | avoid NSAID |
| | | | | | | Follow up in 1 |
| | | | | | | week |
| Appropriate | | Short-course | Start | Short-course | Discontinue or | Resume |
| | | furosemide | acetaminophen, | furosemide | reduce dose of | bisoprolol |
| | | | duloxetine, or | Start SGLT2 | sacubitril/ | Maximize non- |
| | | | tramadol for | inhibitor | valsartan | pharmacologic |
| | | | chronic knee | | Add LAMA | treatment for |
| | | | pain | | Start SGLT2 | osteoarthritis |
| | | | Start SGLT2 | | inhibitor | Start SGLT2 |
| | | | inhibitor | | Low salt | inhibitor |
| | | | | | diet/nutrition | |
| | | | | | consult | |
| Unnecessary | Stress | Add | Add another | Continue | Start loop | Instruct patient |
| | echocardiogram | mineralocorticoid | pain | celecoxib | diuretic | to discontinue |
| | Nuclear | receptor | reliever/NSAID or | Increase | Start | all NSAIDs |
| | myocardial | antagonist | shift celecoxib | furosemide | aldosterone | Start loop |
| | perfusion | Add ivabradine | to nonselective | (from as- | antagonist | diuretic |
| | imaging | Increase | NSAID | needed to | Discuss options | Discontinue |
| | | sacubitril/ | | standing doses) | for | kava kava |
| | | valsartan dose | | Add another | revascularization if | Referral to |
| | | Fibrates | | anti- | amenable | orthopedics for |
| | | Anticoagulation | | hypertensive | Cardiology | osteoarthritis |
| | | Digoxin | | agent: calcium | referral | management |
| | | More testing | | channel | | |
| | | (CAD testing) | | blocker, | | |
| | | Cardiology | | thiazide | | |
| | | referral | | More testing | | |
| | | | | (2D- | | |
| | | | | echocardiogram | | |
| | | | | to recheck | | |
| | | | | ejection | | |
| | | | | fraction; CAD | | |
| | | | | testing) | | |
| | | | | Cardiology | | |
| | | | | referral | | |

Note:
Control group are more likely to order Appropriate tests
BNP = B-type natriuretic peptide
NT-proBNP = N-terminal pro-B-type natriuretic peptide
BP = blood pressure;
CAD = coronary artery disease;
CKD = chronic kidney disease;
COPD = chronic obstructive pulmonary disease;
DDI = drug-drug interaction;
LABA = long-acting beta-2 agonist;
LAMA = long-acting muscarinic antagonist;
NSAID = non-steroidal anti-inflammatory drug;
SGLT2 = sodium-glucose cotransporter-2

### Intervention

Providers are randomly assigned to either the control or intervention arm of the study. After completion of the first (baseline) round of CPVs, intervention group providers are provided educational materials on the CDM test. Education materials include a slide deck, fact sheet, and case studies and are designed to orient the participant to test specifications and interpretation of test results. After viewing educational materials, both intervention and control group participants are asked to complete an additional round of CPVs, with only the intervention group having access to CDM test results. The control group is not exposed to CDM test educational materials, nor granted access to the CDM test.

### Outcome Measures

The primary outcome measure is recognition of NA, DDI, or disease progression in the respective cases. Differences in NA, DDI, and disease progression care made by intervention versus control providers are contrasted. More specifically, the overall quality of care scores after the introduction of the CDM test, as well as scores for the work-up, recognition, and management of patients between control providers (using standard of care diagnostic tools) and intervention providers (with access to CDM test results) are evaluated. Correct management is defined as the following: continuing medication in the case of NA, stopping interacting medications in the case of DDI, or increasing medications, performing further workup, or interventional procedure for disease progression. Counseling patients on the diagnosis is also measured. Secondary outcomes include the effect of provider and clinical practice characteristics on care, cost implications of the CDM test, and identification of the most appropriate use cases of the CDM test.

### Statistical Analysis

Summary statistics are determined for all variables. For categorical dependent outcomes, a chi-square test for single binary independent variables, and a fixed-effects difference-in-difference estimation for multivariate modeling are employed. For analyses involving continuous outcomes, t-tests and linear regression modeling are performed. All analyses are conducted using Stata 15.1 software.

### Results

### Participant Characteristics

One-hundred-and-fifty individuals have completed the first round of data collection and the provider survey. Of these, 11 report that they could not complete the second round of data collection, due to time constraints. In all, 139 participants have completed all six CPV patient cases across the two rounds (Table 16). There are no significant differences in participant characteristics between the intervention and control arms of the study. Approximately 30% of participants identify as male and 70% as female in both groups (p=0.608), and approximately 70% are advanced practice providers (APPs) (p=0.892), with the remainder being physicians. No statistically significant differences between control and intervention arms based on practice setting or practice type are found (p>0.05 for all).

TABLE 16
Baseline participant characteristics.

| | Control | Intervention | P value |
|---|---|---|---|
| n | 70 | 69 | — |

Gender

| | | | |
|---|---|---|---|
| Male | 30.0% | 26.1% | 0.608 |
| Female | 70.0% | 73.9% | |
| APP | 71.4% | 72.5% | 0.892 |

Age Group

| | | | |
|---|---|---|---|
| <40 | 30.0% | 33.3% | 0.293 |
| 40-55 | 48.6% | 36.2% | |
| >55 | 21.4% | 30.4% | |

Practice Setting

| | | | |
|---|---|---|---|
| Urban | 33.8% | 27.9% | 0.683 |
| Suburban | 58.8% | 61.8% | |
| Rural | 7.4% | 10.3% | |

Region

| | | | |
|---|---|---|---|
| Northeast | 11.8% | 10.3% | 0.774 |
| South | 58.8% | 54.4% | |
| Midwest | 11.8% | 10.3% | |
| West | 17.7% | 25.0% | |

Practice Type (could select more than one)

| | | | |
|---|---|---|---|
| ACO | 33.8% | 19.1% | 0.052 |
| Solo | 1.5% | 1.5% | 1.000 |
| Group | 69.1% | 80.9% | 0.113 |
| Integrated Delivery System | 7.4% | 2.9% | 0.244 |
| HMO | 5.9% | 10.3% | 0.345 |
| Other | 4.4% | 0.0% | 0.080 |

Payer

| | | | |
|---|---|---|---|
| Medicare | 39.5% | 39.3% | 0.959 |
| Medicaid | 9.8% | 8.2% | 0.480 |
| Commercial | 42.4% | 46.0% | 0.248 |
| Self | 6.9% | 6.1% | 0.444 |
| Other | 1.3% | 0.3% | 0.077 |
| Employed by Practice | 98.5% | 95.6% | 0.310 |

Participant in CMS Quality Program

| | | | |
|---|---|---|---|
| Yes | 58.8% | 50.0% | 0.506 |
| No | 7.4% | 11.8% | |
| Don't know | 33.8% | 38.2% | |

### Baseline Identification and Treatment of the Primary Cause of Presenting Symptoms

At baseline, participants correctly identify the cause(s) of their patients' symptoms in 35.300 of cases. In round one, there is no statistically significant difference between study arms (control: 35.7% vs intervention: 34.8%, p=0.842) (Table 17), regardless of primary cause of symptoms (NA, DDI, or disease progression). Notably, while less than 20% of participants correctly identify NA in variant A (control: 18.6% vs intervention: 16.4%, p=0.740) or DDI in variant B (control: 21.1% vs intervention 17.4%, p=0.575) as a primary cause, more than two-thirds are able to identify disease progression in variant C (control: 68.1% vs intervention 69.0%, p=0.909). Intervention arm participants perform similarly to control arm participants in correctly treating the primary cause of symptoms across all case variants (p=0.144) (Table 18a).

TABLE 17
Correct identification of presenting symptoms, overall and by case variant.

| | | | | | Difference-in- |
|---|---|---|---|---|---|
| | | | | Difference-in- | Difference, |
| | Round 1 | Round 2 | P value | Difference | P value |
| All Cases | | | | | |
| Control | 35.7% | 32.9% | 0.537 | | |
| Intervention | 34.8% | 69.6% | <0.001 | 37.6% | <0.001 |
| P value | 0.842 | <0.001 | | | |
| Variant A - Medication Non-Adherence | | | | | |
| Control | 18.6% | 10.0% | 0.147 | | |
| Intervention | 16.4% | 80.0% | <0.001 | 72.2% | <0.001 |
| P value | 0.740 | <0.001 | | | |
| Variant B - Drug-Drug Interaction | | | | | |
| Control | 21.1% | 18.6% | 0.704 | | |
| Intervention | 17.4% | 52.9% | <0.001 | 38.1% | 0.002 |
| P value | 0.575 | <0.001 | | | |
| Variant C - Disease Progression | | | | | |
| Control | 68.1% | 70.0% | 0.810 | | |
| Intervention | 69.0% | 75.4% | 0.402 | 4.5% | 0.664 |
| P value | 0.909 | 0.478 | | | |

TABLE 18
Correct treatment of and advising on primary cause of symptoms, overall and
by case variant. (a) Correct primary treatment. (b) Advise on symptoms.

| | | | | |
|---|---|---|---|---|
| | | | | Difference-in- |
| | | | Difference-in- | Difference, |
| Round 1 | Round 2 | P value | Difference | P value |

(a) Correct Primary Treatment

| | | | | | |
|---|---|---|---|---|---|
| All Cases | | | | | |
| Control | 29.1% | 35.7% | 0.144 | | |
| Intervention | 35.8% | 46.4% | 0.028 | 4.0% | 0.640 |
| P value | 0.144 | 0.027 | | | |
| Variant A - NA (Continue Medications) | | | | | |
| Control | 24.3% | 25.7% | 0.845 | | |
| Intervention | 26.9% | 34.3% | 0.346 | 6.0% | 0.611 |
| P value | 0.729 | 0.268 | | | |
| Variant B - DDI (Stop Interacting Medications) | | | | | |
| Control | 21.2% | 27.1% | 0.404 | | |
| Intervention | 24.6% | 58.8% | <0.001 | 28.3% | 0.035 |
| P value | 0.621 | >0.001 | | | |
| Variant C - Disease Progression (Revascularization, | | | | | |
| HTN Workup; Increase Medications) | | | | | |
| Control | 42.0% | 54.3% | 0.148 | | |
| Intervention | 54.9% | 46.4% | 0.312 | −20.8% | 0.083 |
| P value | 0.127 | 0.351 | | | |

(b) Advise on Symptoms

| | | | | | |
|---|---|---|---|---|---|
| All Cases | | | | | |
| Control | 21.4% | 23.3% | 0.640 | | |
| Intervention | 24.2% | 34.3% | 0.023 | 8.3% | 0.230 |
| P value | 0.507 | 0.013 | | | |
| Variant A - NA | | | | | |
| Control | 4.3% | 1.4% | 0.310 | | |
| Intervention | 3.0% | 32.9% | <0.001 | 32.7% | 0.005 |
| P value | 0.685 | <0.001 | | | |
| Variant B - Drug-Drug Interactions | | | | | |
| Control | 1.4% | 1.4% | 0.992 | | |
| Intervention | 1.5% | 2.9% | 0.551 | 1.5% | 0.707 |
| P value | 0.984 | 0.542 | | | |
| Variant C - Disease Progression | | | | | |
| Control | 59.4% | 67.1% | 0.345 | | |
| Intervention | 66.2% | 66.7% | 0.953 | −7.3% | 0.535 |
| P value | 0.407 | 0.952 | | | |

### Impact of CDM Testing on Diagnosis & Treatment of NA, DDI, and Disease Progression

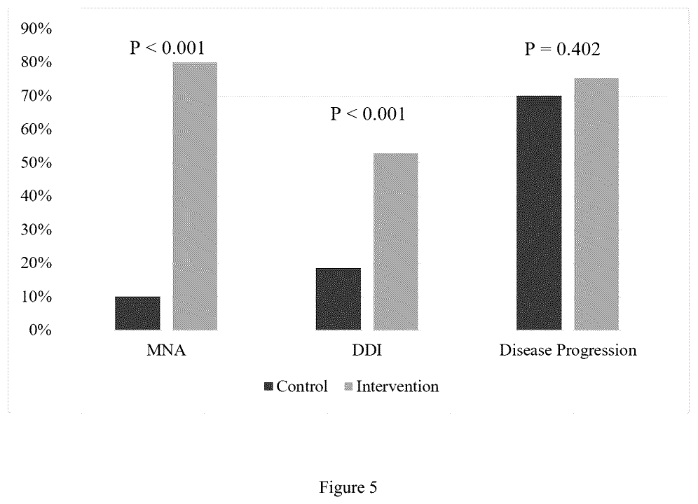

After introducing the CDM test to the intervention group, clinical practice in the second round of data collection is compared (Table 17 and FIG. 5). The intervention group is significantly more likely to make the diagnosis of NA (control: 10.0% vs intervention: 80.0%, p<0.001). They are also significantly more likely to make the diagnosis of DDI (control: 18.6% vs intervention: 52.9%, p<0.001). Participants in both the control and intervention groups who missed the NA and DDI diagnoses believe that symptoms are due to disease progression, and subsequently would increase current medication dosage, switch to a new medication, or add medications. By the second round, this is mostly accounted for by the control group. There is, however, no significant difference between the intervention and control groups in making the diagnosis of disease progression (control: 70.0% vs intervention: 75.4%).

By case type, providers in the intervention group are significantly more likely to correctly diagnose across all primary causes of symptoms (NA, DDI, and/or disease progression) for the HFrEF case type versus the control group (O.R. 3.2, p=0.024) and are slightly, but not significantly, more likely to do so in the other two case types (HFpEF: O.R. 1.4, p=0.481; COPD: O.R. 2.1, p=0.139). In aggregate, the fixed effects difference-in-difference model shows that CDM test use improved diagnostic accuracy across all case types by +37.6% (p<0.001) (Table 17).

By variant, the greatest improvement is seen in diagnosing NA (+72.2%, p<0.001) then DDI cases (+38.1%, p=0.002), and although there is a small improvement in diagnosing disease progression (+4.5 difference-in-difference), this trend is not statistically significant (p=0.664).

In reference to management, the CDM test significantly increases providers' rates of discontinuing interacting medication(s) in the DDI cases (difference-in-difference=+28.3%, p=0.035), but does not affect the decision to continue medications in the NA cases or increase medication/pursue further intervention in the disease progression cases (Table 18a). In contrast, counseling patients significantly improves in NA cases (+32.7%, p=0.005) but not DDI or disease progression cases (Table 18b). Taken together, there is no significant difference overall in treatment by case type in offering the correct treatment (p>0.05 for all) or for counseling (p>0.05 for all).

### Correct Identification of NA, DDI, or Disease Progression

Participants who correctly identify the underlying primary cause of the symptoms are significantly more likely to provide the appropriate treatment (advising on the importance of maintaining their medication regimen for NA cases; stopping the interacting medication(s) in the DDI cases; or ordering revascularization, increased workup, or changes/increases in medications in the disease progression cases) (50.4% vs. 24.1%, p<0.001) across all case variants. Interestingly, those who identify the correct cause are significantly more likely to provide the appropriate treatment within the DDI variant (66.7% vs. 12.4%, p<0.001), non-significantly more likely within the disease progression variant (53.1% vs. 38.6%), and slightly less likely (nonsignificant) within the NA variant (20.8% vs. 30.1%, p=0.361).

A multivariable logistic regression model is conducted to determine whether providers who correctly identify the underlying cause of their patients' symptoms choose appropriate treatment. Results show that those who do are 3.5 times as likely to provide the correct primary treatment (95% C.I 2.5-4.7) compared to those who do not. While there is still some advantage to being in the intervention group (O.R. 1.4), this difference is not significant (95% C.I. 0.9-2.2). The advantage of correctly identifying the underlying cause of symptoms is proven true for both DDI (O.R. 12.5, 95% C.I. 6.5-24.2) and disease progression variants (O.R. 2.3, 95% C.I. 1.3-3.9). However, among NA variants, the opposite is proven to be true, with those identifying the correct cause of symptoms being less likely to correctly treat their patients (O.R. 0.8, 95% C.I. 0.4-1.6).

Improvement with CDM Test

Next, those who originally list an incorrect diagnosis in the baseline round but subsequently identify the correct diagnosis in the second round is looked at. These participants only indicate the correct primary treatment 29.6% of the time in the baseline round (when they provide an incorrect diagnosis), in comparison to 67.2% of the time in the second round (when they provide the correct diagnosis) (p<0.001). This remains significant across all case variants (NA baseline: 32.7% vs. second round: 51.9%, p=0.047; DDI baseline: 19.5% vs. second round: 80%, p<0.001; disease progression: baseline: 37.5% vs. second round: 75.8%, p=0.002).

### Comparing Physician and APP Performance in the Intervention Arm

When the performance of advanced practice providers (APPs) vs physicians is investigated at baseline, physicians are about as likely as APPs to correctly identify the cause of their patients' symptoms (O.R. 1.3, 95% C.I. 0.7-2.4) and provide the correct primary treatment (O.R. 0.7, 95% C.I. 0.4-1.2). This trend is consistent across all three variants. After introduction of the CDM test, intervention arm physicians' diagnostic performance is improved in identifying the underlying causes of symptoms when compared to APPs (O.R. 1.7), although this is nonsignificant (95% CI 0.7-4.0). Physicians are significantly better at ordering the appropriate primary treatment (O.R. 3.2, 95% C.I. 1.4-7.5). Specifically, physicians in the intervention group are more likely to correctly order the proper treatment within disease progression cases (revascularization, HTN workup, or increase medications) (O.R. 7.2, 95% C.I. 1.6-30.7). Physicians perform slightly worse across all cases at counseling patients on the cause of their symptoms (O.R. 0.6), although this also is nonsignificant (95% C.I. 0.3-1.6).

### Discussion

Chronic disease management is a consequential challenge currently facing primary care providers. When patients present for a routine visit with worsening symptoms, it is incumbent on providers to check for NA and DDIs before concluding their disease has progressed. It has been reported that this is not routinely done (Valdenor et al., “Clinical Variation in the Treatment Practices for Medication Nonadherence, Drug-Drug Interactions, and Recognition of Disease Progression in Patients with Chronic Cardiometabolic Diseases: A Cross-Sectional Patient Simulation Study among Primary Care Physicians,” Int J Clin Pract. 2022:6450641 (2022); Peabody et al., “Drug-Drug Interaction Assessment and Identification in the Primary Care Setting,” J Clin Med Res. 10(11):806-14 (2018)). In a recent study of 246 primary care physicians, participants correctly identified, as a cause of symptoms, NA in just 3.6% of cases, DDIs in 8.9% of cases, and disease progression in 30.3% of cases (Valdenor et al., “Clinical Variation in the Treatment Practices for Medication Nonadherence, Drug-Drug Interactions, and Recognition of Disease Progression in Patients with Chronic Cardiometabolic Diseases: A Cross-Sectional Patient Simulation Study among Primary Care Physicians,” Int J Clin Pract. 2022:6450641 (2022)).

In this randomized controlled trial among simulated patients, intervention providers are given results from a simple test that detects NA and DDIs. Access to the CDM test has resulted in post-intervention round participants markedly increasing their rate of correctly identifying NA, DDI, and disease progression (69.6% in the intervention group compared with 32.9% in the control group). Impressively, NA is diagnosed in 80% of patients among participants with access to the CDM test but only 10% among those without access. Also noteworthy is the improvement in diagnosing DDIs-53% with the CDM test and 19% without. Disease progression is diagnosed with a similar accuracy between groups both at baseline and post-intervention, demonstrating that providers are more skilled at this task than in identifying NA and DDIs. When the correct diagnosis is made for symptomatic patients, treatment significantly improves, from 29.6% of the time in the baseline round (incorrect diagnosis) to 67.2% of the time in the second round (correct diagnosis), regardless of study arm. This is true for patients with NA, DDI, and disease progression alike. Advanced practice providers and physicians perform similarly, both at baseline and post-intervention at identifying NA and DDI in symptomatic patients.

In this study, the baseline data demonstrate that providers correctly identify NA, DDI, or disease progression in approximately one-third of all patient cases, regardless of provider type (physician or APP). Vitally, participants who correctly identify the primary cause of symptoms are significantly more likely to then order the appropriate treatment—most notably, stopping medications with clinically significant drug interactions. Its consequences for clinical utility cannot be understated.

These findings have broad clinical implications for chronic disease management. Rather than continuing to add medications to a patient's regimen to mitigate a new a symptom, a thoughtful and wholistic review of the system of potentially interacting medications and overall adherence will foster superior management of chronic cardiometabolic diseases. This is precisely what the CDM test appears to have prompted-healthcare practitioners providing improved patient care that reduces the complexities of chronic disease management.

In real-world clinical settings, providers have many competing priorities for the finite time that they have available to spend with each patient (Tai-Seale et al., “Electronic Health Record Logs Indicate That Physicians Split Time Evenly Between Seeing Patients And Desktop Medicine,” Health Aff (Millwood) 36(4):655-62 (2017)). A thorough review of a patient's medication regimen is but one aspect of an appropriate medical evaluation or interaction. There is a conspicuous need for a tool to assist providers in medication management in patients with multiple comorbidities. A simple test for NA and DDIs offers a quality solution to increasing accuracy and reducing undue provider burden for medication management, hence creating care value. It is recently demonstrated that when given the chance to order the CDM test, only 12.4% of providers chose to do so (Peabody et al., “Randomized prospective trial to detect and distinguish between medication nonadherence, drug-drug interactions, and disease progression in chronic cardiometabolic disease,” BMC Prim Care 24(1):100 (2023)). However, those who did perform significantly better in making the appropriate primary NA or DDI diagnosis, 60% vs. 18.9% respectively, than those who did not (p<0.001). This has led to a significant improvement in treatment, 50% vs. 18.2%. Patients will prefer optimal medical management of their chronic disease rather than the addition of unnecessary medications, costly procedures, or continuation of interacting drugs, and that broader knowledge of the availability of objective testing that CDM test provides will result in an increase in medically necessary utilization. The CDM test is a feasible, simply administered, office-based test available to chronic disease patients as a tool to help guide and improve patient care.

### Conclusion

Identification of NA and DDIs is one of many clinical challenges that providers face when treating chronic disease patients who remain symptomatic. The introduction of an objective, saliva-based test has demonstrated significant improvement of diagnostic accuracy for NA and disease progression, identification of DDIs, and subsequent treatment in an experimental study. The CDM test has the potential to impact how follow-up patients with chronic disease are evaluated, and thereby improve patient outcomes and value in real-world settings.

1. A method for treating a chronic disease in a patient in need thereof, comprising:
obtaining an oral fluid sample from the patient;
analyzing the oral fluid for analytes of one or more drug classes selected from the group consisting of antiarrhythmics; antidepressants and antipsychotics; antiemetics and gastric reflux therapies; antiepileptics; antimicrobials and antivirals;
cardiovascular; chemotherapeutic agents; cognitive enhancement agents; diabetes mellitus (DM); diabetes mellitus type 2 (DM2); nonsteroidal anti-inflammatory drugs (NSAIDs); skeletal muscle relaxant; foods and supplements; and miscellaneous drugs;
identifying a list of drugs recently ingested by the patient based on the analyzing step;
determining whether any drug-drug interaction (DDI) is present between the drugs in the list; and
if DDI is present, presenting a first treatment plan to the patient; or
if DDI is not present, presenting a second treatment plan to the patient.

2. The method of claim 1, wherein the list of drugs comprises prescription drugs, non-prescription drugs, and other miscellaneous substances.

3. The method of claim 1, further comprising administering an alternative intervention to the patient, wherein the alternative intervention does not interact with the remaining drugs in the list.

4. (canceled)

5. (canceled)

6. The method of claim 1, wherein the first treatment plan comprises one or more treatment options selected from the group consisting of implementing a monitoring plan for the patient, decreasing the dosage of one or more drugs determined as interacting with another drug in the list, increasing the dosage of one or more drugs determined as interacting with another drug in the list, discontinuing administration of one or more drugs determined as interacting with another drug in the list, continuing administration of the remaining drugs in the list, and a combination thereof.

7. The method of claim 1, wherein the second treatment plan comprises one or more treatment options selected from the group consisting of implementing one or more assessments for the presenting signs and symptoms experienced by the patient, continuing administration of the list of drugs, and a combination thereof.

8. The method of claim 1, wherein the analyzing step is performed by mass spectrometry.

9. The method of claim 8, wherein the mass spectrometry is high-performance liquid chromatography-tandem mass spectrometry (LC-MS/MS).

10.-21. (canceled)

22. The method of claim 1, wherein the analytes of the miscellaneous drug class are selected from the group consisting of avanafil, colchicine, cyclosporine, 7-hydroxymethotrexate, methotrexate, tacrolimus, and a combination thereof.

23. The method of claim 1, wherein the chronic disease is selected from the group consisting of chronic kidney disease (CKD), coronary heart disease (CHD), diabetes mellitus (DM), diabetes mellitus type 2 (DM2), chronic heart failure (CHF), stroke, coronary artery disease (CAD), hyperlipidemia, and hypertension.

24.-29. (canceled)

30. The method of claim 1, wherein prior to the treatment, the patient is administered 5 or more drugs concurrently.

31.-62. (canceled)

63. A method for treating a chronic disease in a patient in need thereof, comprising:
obtaining an oral fluid sample from the patient;
analyzing the oral fluid for analytes of one or more drug classes selected from the group consisting of asthma/chronic obstructive pulmonary disease (COPD); cardiovascular; diabetes mellitus (DM); diabetes mellitus type 2 (DM2); and chronic kidney disease (CKD);
identifying a list of drugs recently ingested by the patient based on the analyzing step;
determining if there is medication nonadherence (NA) in the list of drugs by generating a list of drugs with NA and a list of drugs without NA; and
if NA is present, presenting a first treatment plan to the patient; or
if NA is not present, presenting a second treatment plan to the patient.

64. The method of claim 63, wherein the determining step comprises generating compliance calls based on comparing against a prescription drug list.

65. The method of claim 63, wherein the list of drugs comprises prescription drugs, non-prescription drugs, and other miscellaneous substances.

66. The method of claim 63, wherein the first treatment plan comprises one or more treatment options selected from the group consisting of implementing a monitoring plan for the patient, decreasing the dosage of one or more drugs with NA, increasing the dosage one or more drugs with NA, discontinuing administration of one or more drugs with NA, continuing administration of the list of drugs without NA, administering an increased dosage of one or more drugs in the list of drugs, advising the patient about the importance of adherence, providing aids and tools to increase adherence of drugs in the list of drugs with NA, and a combination thereof.

67. The method of claim 63, further comprising administering an alternative intervention to the patient, wherein the alternative intervention comprises administration of one or more new drugs to the patient.

68. (canceled)

69. The method of claim 63, wherein the second treatment plan comprises one or more treatment options selected from the group consisting of implementing one or more assessments for the presenting signs and symptoms experienced by the patient, continuing administration of the list of drugs, and a combination thereof.

70. The method of claim 63, wherein the analyzing step is performed by mass spectrometry.

71. The method of claim 70, wherein the mass spectrometry is high-performance liquid chromatography-tandem mass spectrometry (LC-MS/MS).

72.-82. (canceled)

83. The method of claim 63, wherein prior to the treatment, the patient is administered 5 or more drugs concurrently.

84.-136. (canceled)
